SEQUENCE LISTING (94 sequences)

SEQ ID NO: 1 - DNA, 24 bp, Artificial Sequence
  Synthesized - M1 oligonucleotide
  misc_feature (1)..(1): 5′ Cy3 fluorophore
cttttgaccgcatactcccttagc

SEQ ID NO: 2 - RNA, 24 nt, Artificial Sequence
  Synthesized - M2 oligonucleotide
  misc_feature (1)..(1): 5′ Cy3 fluorophore
cuuuugaccgcauacucccuuagc

SEQ ID NO: 3 - DNA, 24 bp, Artificial Sequence
  Synthesized - M3 oligonucleotide
  misc_feature (1)..(1): 5′ Cy3 fluorophore
  misc_feature (1)..(4): 2′-O-methylated ribonucleotide
  misc_feature (21)..(24): 2′-O-methylated ribonucleotide
cuuutgaccgcatactccctuagc

SEQ ID NO: 4 - RNA, 24 nt, Artificial Sequence
  Synthesized - M4 oligonucleotide
  misc_feature (1)..(1): 5′ Cy3 fluorophore
  misc_feature (1)..(4): 2′-O-methyl ribonucleotide
  misc_feature (21)..(24): 2′-O-methyl ribonucleotide
cuuuugaccgcauacucccuuagc

SEQ ID NO: 5 - RNA, 24 nt, Artificial Sequence
  Synthesized - M5 oligonucleotide
  misc_feature (1)..(1): 5′ Cy3 fluorophore
  misc_feature (1)..(24): 2′-O-methyl ribonucleotide
cuuuugaccgcauacucccuuagc

SEQ ID NO: 6 - DNA, 30 bp, Artificial Sequence
  Synthesized - M6 oligonucleotide
  misc_feature (1)..(1): 5′ Cy3 fluorophore
agcaggggagcgccttcagcctctcggcca

SEQ ID NO: 7 - RNA, 30 nt, Artificial Sequence
  Synthesized - M7 oligonucleotide
  misc_feature (1)..(1): 5′ Cy3 fluorophore
agcaggggagcgccuucagccucucggcca

SEQ ID NO: 8 - RNA, 30 nt, Artificial Sequence; identical to SEQ ID NO: 7
  Synthesized - M8 oligonucleotide
  misc_feature (1)..(1): 5′ Cy3 fluorophore
  misc_feature (1)..(30): 2′-O-methylated ribonucleotide

SEQ ID NO: 9 - RNA, 24 nt, Artificial Sequence
  Synthesized - M5-1 oligonucleotide
  misc_feature (1)..(24): 2′-O-methylated ribonucleotide
cuuuugaccgcauacucccuuagc

SEQ ID NO: 10 - RNA, 24 nt, Artificial Sequence
  Synthesized - M5-2 oligonucleotide
  misc_feature (1)..(7): 2′-O-methylated ribonucleotide
  misc_feature (10)..(11): 2′-O-methylated ribonucleotide
  misc_feature (13)..(14): 2′-O-methylated ribonucleotide
  misc_feature (16)..(19): 2′-O-methylated ribonucleotide
  misc_feature (22)..(24): 2′-O-methylated ribonucleotide
cuuuugaccgcauacucccuuagc

SEQ ID NO: 11 - RNA, 24 nt, Artificial Sequence
  Synthesized - M5-3 oligonucleotide
  misc_feature (1)..(4): 2′-O-methylated ribonucleotide
  misc_feature (6)..(7): 2′-O-methylated ribonucleotide
  misc_feature (11)..(11): 2′-O-methylated ribonucleotide
  misc_feature (16)..(16): 2′-O-methylated ribonucleotide
  misc_feature (18)..(18): 2′-O-methylated ribonucleotide
  misc_feature (22)..(23): 2′-O-methylated ribonucleotide
cuuuugaccgcauacucccuuagc

SEQ ID NO: 12 - RNA, 22 nt, Artificial Sequence
  Synthesized - M5T1 oligonucleotide
  misc_feature (1)..(22): 2′-O-methylated ribonucleotide
  misc_feature (22)..(22): 3′ Amino Modifier
uuugaccgcauacucccuuagc

SEQ ID NO: 13 - RNA, 19 nt, Artificial Sequence
  Synthesized - M5T2 oligonucleotide
  misc_feature (1)..(19): 2′-O-methylated ribonucleotide
  misc_feature (19)..(19): 3′ Amino Modifier
gaccgcauacucccuuagc

SEQ ID NO: 14 - RNA, 20 nt, Artificial Sequence
  Synthesized - M5T3 oligonucleotide
  misc_feature (1)..(20): 2′-O-methylated ribonucleotide
  misc_feature (20)..(20): 3′ Amino Modifier
cuuuugaccgcauacucccu

SEQ ID NO: 15 - RNA, 13 nt, Artificial Sequence
  Synthesized - M5T4 oligonucleotide
  misc_feature (1)..(13): 2′-O-methylated ribonucleotide
  misc_feature (13)..(13): 3′ Amino Modifier
cuuuugaccgcau

SEQ ID NO: 16 - RNA, 24 nt, Artificial Sequence
  Synthesized - MF5 oligonucleotide
  misc_feature (1)..(24): 2′-fluoro ribonucleotide
cuuuugaccgcauacucccuuagc

SEQ ID NO: 17 - RNA, 24 nt, Artificial Sequence
  Synthesized - R1 oligonucleotide
  misc_feature (1)..(24): 2′-O-methylated ribonucleotide
aggaggggcucguuauauccauuu

SEQ ID NO: 18 - RNA, 22 nt, Artificial Sequence
  Synthesized - R2 oligonucleotide
  misc_feature (1)..(22): 2′-O-methylated ribonucleotide
ccugcaauuagcccuuaggagg

SEQ ID NO: 19 - RNA, 25 nt, Artificial Sequence
  Synthesized - R3 oligonucleotide
  misc_feature (1)..(25): 2′-O-methylated ribonucleotide
aucgaaccugcaauuagcccuuagg

SEQ ID NO: 20 - RNA, 24 nt, Artificial Sequence
  Synthesized - R4 oligonucleotide
  misc_feature (1)..(24): 2′-O-methylated ribonucleotide
ugguguccccugcaggaaucgaac

SEQ ID NO: 21 - RNA, 21 nt, Artificial Sequence
  Synthesized - L1 oligonucleotide
  misc_feature (1)..(1): 5′ Cy3 fluorophore
  misc_feature (1)..(21): 2′-O-methylated ribonucleotide
gagaucacaccugcuuagcag

SEQ ID NO: 22 - RNA, 26 nt, Artificial Sequence
  Synthesized - L2 oligonucleotide
  misc_feature (1)..(1): 5′ Cy3 fluorophore
  misc_feature (1)..(26): 2′-O-methylated ribonucleotide
uuagcaggcaggcgccuuaaccacuc

SEQ ID NO: 23 - RNA, 20 nt, Artificial Sequence
  Synthesized - L3 oligonucleotide
  misc_feature (1)..(20): 2′-O-methylated ribonucleotide
gcuuagcaggcaggcgccuu

SEQ ID NO: 24 - RNA, 21 nt, Artificial Sequence
  Synthesized - L4 oligonucleotide
  misc_feature (1)..(21): 2′-O-methylated ribonucleotide
uggcgcaaacggaaggguucg

SEQ ID NO: 25 - RNA, 22 nt, Artificial Sequence
  Synthesized - L5 oligonucleotide
  misc_feature (1)..(22): 2′-O-methylated ribonucleotide
uuaaccacucggccacauuugc

SEQ ID NO: 26 - RNA, 21 nt, Artificial Sequence
  Synthesized - L6 oligonucleotide
  misc_feature (1)..(21): 2′-O-methylated ribonucleotide
uucgaaccuucgcgugagauc

SEQ ID NO: 27 - RNA, 52 nt, Artificial Sequence
  Synthesized - oligo E. coli tRNASerGCU-F1
gcuuuugaccgcauacucccuuagcaggggagcgccuucagccucucggcca

SEQ ID NO: 28 - RNA, 41 nt, Artificial Sequence
  Synthesized - oligo E. coli tRNASerGGA-F6
gcuuuccaggcgugcuccuucagccacucggacacgucacc

SEQ ID NO: 29 - RNA, 49 nt, Artificial Sequence
  Synthesized - oligo E. coli tRNASerUGA-F2
gcuuucgggucgccgguuuucaagaccggugccuucaaccgcucggcca

SEQ ID NO: 30 - RNA, 42 nt, Artificial Sequence
  Synthesized - oligo E. coli tRNAArgCCG-F1
gaccucugccuccggagggcagcgcucuauccagcugagcua

SEQ ID NO: 31 - RNA, 42 nt, Artificial Sequence
  Synthesized - oligo tRNAiMetCAU-F1
gaccuucggguuaugagcccgacgagcuaccaggcugcucca

SEQ ID NO: 32 - RNA, 93 nt, Escherichia coli
  tRNA (1)..(93): E. coli tRNASerGCU
ggugagguggccgagaggcugaaggcgcuccccugcuaagggaguaugcggucaaaagcugcauccgggguucgaauccccgccucaccgcca

SEQ ID NO: 33 - RNA, 75 nt, Escherichia coli
  tRNA (1)..(75): wild type (wt) E. coli tRNAGlyUCC
gcgggcaucguauaauggcuauuaccucagccuuccaagcugaugaugcggguucgauucccgcugcccgcucca

SEQ ID NO: 34 - RNA, 88 nt, Escherichia coli
  tRNA (1)..(88): wt E. coli tRNASerGGA
ggugagguguccgaguggcugaaggagcacgccuggaaaguguguauacggcaacguaucggggguucgaaucccccccucaccgcca

SEQ ID NO: 35 - RNA, 88 nt, Escherichia coli
  tRNA (1)..(88): wt E.coli tRNASerUGA
ggaaguguggccgagcgguugaaggcaccggucuugaaaaccggcgacccgaaaggguuccagaguucgaaucucugcgcuuccgcca

SEQ ID NO: 36 - RNA, 75 nt, Escherichia coli
  tRNA (1)..(75): E. coli tRNAArgCCU
guccucuuaguuaaauggauauaacgagccccuccuaagggcuaauugcagguucgauuccugcaggggacacca

SEQ ID NO: 37 - RNA, 77 nt, Escherichia coli
  tRNA (1)..(77): wt E.coli tRNAArgCCG
gcgcccguagcucagcuggauagagcgcugcccuccggaggcagaggucucagguucgaauccugucgggcgcgcca

SEQ ID NO: 38 - RNA, 77 nt, Escherichia coli
  tRNA (1)..(77): wt E.coli tRNAiMetCAU
cgcgggguggagcagccugguagcucgucgggcucauaacccgaaggucgucgguucaaauccggcccccgcaacca

SEQ ID NO: 39 - RNA, 81 nt, Leishmania tarentolae
  tRNA (1)..(81): wt L. tarentolae (LTE) tRNASerGCU
gcaaauguggccgagugguuaaggcgccugccugcuaagcaggugugaucucacgcgaagguucgaacccuuccguuugcg

SEQ ID NO: 40 - RNA, 84 nt, Leishmania tarentolae
  tRNA (1)..(84): wt L. tarentolae (LTE) tRNASerGCU 2
gcaaacguggccgagugguuaaggcgccugccugcuaagcaggugugaucucacgcgaagguucgaacccuuccguuugcgcca

SEQ ID NO: 41 - RNA, 84 nt, Leishmania tarentolae
  tRNA (1)..(84): wt L. tarentolae (LTE) tRNASerAGA
gucaccauacccaagugguuacggggacugacuagaaaucaguugcgaucucgcgcgcagguucgaauccugcuggugacgcca

SEQ ID NO: 42 - RNA, 84 nt, Leishmania tarentolae
  tRNA (1)..(84): wt L. tarentolae (LTE) tRNASerCGA
gucaccauacccaagugguuacggggguugacucgaaaucaacugcgaucucgcgcgcagguucgaauccugcuggugacgcca

SEQ ID NO: 43 - RNA, 84 nt, Leishmania tarentolae
  tRNA (1)..(84): wt L. tarentolae (LTE) tRNASerUGA
gucaacauacccaagugguuacgggguuugacuugaaaucaaaugcgaucucgcgcgcagguucgaacccugcuguugacgcca

SEQ ID NO: 44 - RNA, 74 nt, Leishmania tarentolae
  tRNA (1)..(74): wt LTE tRNAGlyCCC
gcgccgcuggucuaguggcaugaugguacccucccaagguauugacccggguucgauucccgggcggcgcacca

SEQ ID NO: 45 - RNA, 75 nt, Artificial Sequence
  Synthesized - synthetic E. coli tRNAGlyGCU
  tRNA (1)..(75): synthetic E. coli tRNAGlyGCU
gcgggcaucguauaauggcuauuaccucagccugcuaagcugaugaugcggguucgauucccgcugcccgcucca

SEQ ID NO: 46 - RNA, 74 nt, Artificial Sequence
  Synthesized - synthetic LTE tRNAGlyGCU
  tRNA (1)..(74): synthetic LTE tRNAGlyGCU
gcgccgcuggucuaguggcaugaugguacccugcuaagguauugacccggguucgauucccgggcggcgcacca

SEQ ID NO: 47 - RNA, 77 nt, Artificial Sequence
  Synthesized - synthetic tRNAAzF2ACU
  tRNA (1)..(77): synthetic tRNAAzF2ACU
ccggccguaguucagcagggcagaacggcggacuacuaauccgcauggcauggguucaaaucccaucggccggacca

SEQ ID NO: 48 - RNA, 77 nt, Artificial Sequence
  Synthesized - synthetic tRNAAzF2GCU
  tRNA (1)..(77): synthetic tRNAAzF2GCU
ccggccguaguucagcagggcagaacggcggacugcuaauccgcauggcauggguucaaaucccaucggccggacca

SEQ ID NO: 49 - RNA, 72 nt, Artificial Sequence
  Synthesized - synthetic tRNAPylACU
  tRNA (1)..(72): synthetic tRNAPylACU
ggaaaccugaucauguagaucgaacggacuacuaauccguucagccggguuagauucccgggguuuccgcca

SEQ ID NO: 50 - RNA, 72 nt, Artificial Sequence
  Synthesized - synthetic tRNAPylGCU
  tRNA (1)..(72): synthetic tRNAPylGCU
ggaaaccugaucauguagaucgaacggacugcuaauccguucagccggguuagauucccgggguuuccgcca

SEQ ID NO: 51 - DNA, 17 bp, Artificial Sequence
  Synthesized - T7 promoter
taatacgactcactata

SEQ ID NO: 52 - DNA, 122 bp, Artificial Sequence
  Synthesized - SITS sequence
tttattttattttattttattttattttattttattttattttattttattttaattaattttatttaaccatgacagtaatgtataaagtctgtaaagacattaaacacgtaagtgaaacc

SEQ ID NO: 53 - protein, 17 aa, Artificial Sequence
  Synthesized - SITS protein sequence
MTVMYKVCKDIKHVSET

SEQ ID NO: 54 - DNA, 807 bp, Artificial Sequence
  Synthesized - 2AGC codon eGFP ORF
atgacagtaatgtataaagtctgtaaagacattaaacacgtaagtgaaaccatggagatcagcagcaagggcgaggagctgttcaccggggtggtgcccatcctggtcgagctggacggcgacgtaaacggccacaagttctccgtgtccggcgagggcgagggcgatgccacctacggcaagctgaccctgaagttcatctgcaccaccggcaagctgcccgtgccctggcccaccctcgtgaccaccctgacctacggcgtgcagtgcttctcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaactcccacaacgtctatatcatggccgacaagcagaagaacggcatcaaggtgaacttcaagatccgccacaacatcgaggacggctccgtgcagctcgccgaccactaccagcagaacacccccatcggcgacggccccgtgctgctgcccgacaaccactacctgtccacccagtccgccctgtccaaagaccccaacgagaagcgcgatcacatggtcctgctggagttcgtgaccgccgccgggatcactctcggcatggacgagctatacaaggagcagaagctgatctccgaggaggatctgtaa

SEQ ID NO: 55 - DNA, 3409 bp, Artificial Sequence
  Synthesized - 2AGC DNA template sequence
gacgtctaatacgactcactatagggacatcttaagtttattttattttattttattttattttattttattttattttattttattttaattaattttatttaaccatgacagtaatgtataaagtctgtaaagacattaaacacgtaagtgaaaccatggagatcagcagcaagggcgaggagctgttcaccggggtggtgcccatcctggtcgagctggacggcgacgtaaacggccacaagttctccgtgtccggcgagggcgagggcgatgccacctacggcaagctgaccctgaagttcatctgcaccaccggcaagctgcccgtgccctggcccaccctcgtgaccaccctgacctacggcgtgcagtgcttctcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaactcccacaacgtctatatcatggccgacaagcagaagaacggcatcaaggtgaacttcaagatccgccacaacatcgaggacggctccgtgcagctcgccgaccactaccagcagaacacccccatcggcgacggccccgtgctgctgcccgacaaccactacctgtccacccagtccgccctgtccaaagaccccaacgagaagcgcgatcacatggtcctgctggagttcgtgaccgccgccgggatcactctcggcatggacgagctatacaaggagcagaagctgatctccgaggaggatctgtaagcggccgccctcctcctcctttcttgttcctttcacgtcgccttctcggttgtagctggcagacgacgagtcttacttttacgtgtacttctctatagatgatgtatgatctctctgcatgcgtgttcgtgcatgtgtccgtgtgttgggtacgcgtggtaccctgcaggaaggaagctgagttggctgctgccaccgctgagcaataactagtaattactagcataaccccttggggcctctaaacgggtcttgagggggttttttgctgaaaggaggacagctgatgattgtcatgcttgccatctgttttcttgcaaggtcagaggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaggatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctagttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 56 - protein, 268 aa, Artificial Sequence
  Synthesized - translated protein sequence from 2AGC template
MTVMYKVCKDIKHVSETMEISSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEQKLISEEDL

SEQ ID NO: 57 - protein, 251 aa, Artificial Sequence
  Synthesized - translated eGFP protein from 2AGC template (i.e., excluding 17aas from SITS)
MEISSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEQKLISEEDL

SEQ ID NO: 58 - DNA, 122 bp, Artificial Sequence
  Synthesized - modified SITS* DNA sequence
tttattttattttattttattttattttattttattttattttattttattttaattaattttatttaaccatgacagtaatgtataaagtctgtaaagacattaaacacttaggtgaaacc

SEQ ID NO: 59 - protein, 17 aa, Artificial Sequence
  Synthesized - modified SITS* protein sequence
MTVMYKVCKDIKHLGET

SEQ ID NO: 60 - DNA, 804 bp, Artificial Sequence
  Synthesized - 1AGC codon eGFP ORF
atgacagtaatgtataaagtctgtaaagacattaaacacttaggtgaaaccatggagatcagcaagggagaggagctgttcacaggagtggtgccgatcctggtagagctggacggagacgtaaacgggcacaagttctcggtgtcaggagagggagagggagatgccacgtacgggaagctgacactgaagttcatctgcacgacaggaaagctgccagtaccgtggccgacgctggtgacgacactgacgtacggagtacagtgcttctcacggtacccagaccacatgaagcagcacgacttcttcaagtcggctatgccagaagggtacgtacaggagagaacgatcttcttcaaggacgacggaaactacaagacacgggctgaggtgaagttcgagggagacacgctggtgaacaggatcgagctgaagggaatcgacttcaaggaggacggaaacatcctgggacacaagctggagtacaactacaactcgcacaacgtatatatcatggccgacaagcagaagaacgggatcaaggtgaacttcaagatccggcacaacatcgaggacggatcggtacagctggctgaccactaccagcagaacacaccgatcggagacggaccagtactgctgccagacaaccactacctgtcgacgcagtcagctctgtcgaaagacccgaacgagaagagagatcacatggtactgctggagttcgtgacagccgctggaatcacgctggggatggacgagctgtacaaggagcagaagctgatctcggaggaggatctgtaa

SEQ ID NO: 61 - DNA, 3406 bp, Artificial Sequence
  Synthesized - 1AGC codon template DNA sequence
gacgtctaatacgactcactatagggacatcttaagtttattttattttattttattttattttattttattttattttattttattttaattaattttatttaaccatgacagtaatgtataaagtctgtaaagacattaaacacttaggtgaaaccatggagatcagcaagggagaggagctgttcacaggagtggtgccgatcctggtagagctggacggagacgtaaacgggcacaagttctcggtgtcaggagagggagagggagatgccacgtacgggaagctgacactgaagttcatctgcacgacaggaaagctgccagtaccgtggccgacgctggtgacgacactgacgtacggagtacagtgcttctcacggtacccagaccacatgaagcagcacgacttcttcaagtcggctatgccagaagggtacgtacaggagagaacgatcttcttcaaggacgacggaaactacaagacacgggctgaggtgaagttcgagggagacacgctggtgaacaggatcgagctgaagggaatcgacttcaaggaggacggaaacatcctgggacacaagctggagtacaactacaactcgcacaacgtatatatcatggccgacaagcagaagaacgggatcaaggtgaacttcaagatccggcacaacatcgaggacggatcggtacagctggctgaccactaccagcagaacacaccgatcggagacggaccagtactgctgccagacaaccactacctgtcgacgcagtcagctctgtcgaaagacccgaacgagaagagagatcacatggtactgctggagttcgtgacagccgctggaatcacgctggggatggacgagctgtacaaggagcagaagctgatctcggaggaggatctgtaagcggccgccctcctcctcctttcttgttcctttcacgtcgccttctcggttgtagctggcagacgacgagtcttacttttacgtgtacttctctatagatgatgtatgatctctctgcatgcgtgttcgtgcatgtgtccgtgtgttgggtacgcgtggtaccctgcaggaaggaagctgagttggctgctgccaccgctgagcaataactagtaattactagcataaccccttggggcctctaaacgggtcttgagggggttttttgctgaaaggaggacagctgatgattgtcatgcttgccatctgttttcttgcaaggtcagaggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaggatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctagttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 62 - protein, 267 aa, Artificial Sequence
  Synthesized - translated protein sequence from 1AGC template
MTVMYKVCKDIKHLGETMEISKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEQKLISEEDL

SEQ ID NO: 63 - protein, 250 aa, Artificial Sequence
  Synthesized - translated eGFP protein from 1AGC template (i.e., excluding 17aas from SITS)
MEISKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEQKLISEEDL

SEQ ID NO: 64 - DNA, 804 bp, Artificial Sequence
  Synthesized - 1AGT codon eGFP ORF
atgacagtaatgtataaagtctgtaaagacattaaacacttaggtgaaaccatggagatcagtaagggcgaggagctgttcaccggggtggtgcccatcctggtcgagctggacggcgacgtaaacggccacaagttctcggtgtcgggcgagggcgagggcgatgccacctacggcaagctgaccctgaagttcatctgcaccaccggcaagctgcccgtgccctggcccaccctggtgaccaccctgacctacggcgtgcagtgcttctcgcggtaccccgaccacatgaagcagcacgacttcttcaagtcggccatgcccgaaggctacgtccaggagcggaccatcttcttcaaggacgacggcaactacaagacccgggccgaggtgaagttcgagggcgacaccctggtgaaccggatcgagctgaagggcatcgacttcaaggaggacggcaacatcctggggcacaagctggagtacaactacaactcgcacaacgtctatatcatggccgacaagcagaagaacggcatcaaggtgaacttcaagatccggcacaacatcgaggacggctcggtgcagctggccgaccactaccagcagaacacccccatcggcgacggccccgtgctgctgcccgacaaccactacctgtcgacccagtcggccctgtcgaaagaccccaacgagaagcgggatcacatggtcctgctggagttcgtgaccgccgccgggatcactctgggcatggacgagctatacaaggagcagaagctgatctcggaggaggatctgtaa

SEQ ID NO: 65 - DNA, 3406 bp, Artificial Sequence
  Synthesized - 1AGT codon template DNA sequence
gacgtctaatacgactcactatagggacatcttaagtttattttattttattttattttattttattttattttattttattttattttaattaattttatttaaccatgacagtaatgtataaagtctgtaaagacattaaacacttaggtgaaaccatggagatcagtaagggcgaggagctgttcaccggggtggtgcccatcctggtcgagctggacggcgacgtaaacggccacaagttctcggtgtcgggcgagggcgagggcgatgccacctacggcaagctgaccctgaagttcatctgcaccaccggcaagctgcccgtgccctggcccaccctggtgaccaccctgacctacggcgtgcagtgcttctcgcggtaccccgaccacatgaagcagcacgacttcttcaagtcggccatgcccgaaggctacgtccaggagcggaccatcttcttcaaggacgacggcaactacaagacccgggccgaggtgaagttcgagggcgacaccctggtgaaccggatcgagctgaagggcatcgacttcaaggaggacggcaacatcctggggcacaagctggagtacaactacaactcgcacaacgtctatatcatggccgacaagcagaagaacggcatcaaggtgaacttcaagatccggcacaacatcgaggacggctcggtgcagctggccgaccactaccagcagaacacccccatcggcgacggccccgtgctgctgcccgacaaccactacctgtcgacccagtcggccctgtcgaaagaccccaacgagaagcgggatcacatggtcctgctggagttcgtgaccgccgccgggatcactctgggcatggacgagctatacaaggagcagaagctgatctcggaggaggatctgtaagcggccgccctcctcctcctttcttgttcctttcacgtcgccttctcggttgtagctggcagacgacgagtcttacttttacgtgtacttctctatagatgatgtatgatctctctgcatgcgtgttcgtgcatgtgtccgtgtgttgggtacgcgtggtaccctgcaggaaggaagctgagttggctgctgccaccgctgagcaataactagtaattactagcataaccccttggggcctctaaacgggtcttgagggggttttttgctgaaaggaggacagctgatgattgtcatgcttgccatctgttttcttgcaaggtcagaggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaggatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctagttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 66 - protein, 267 aa, Artificial Sequence; identical to SEQ ID NO: 62
  Synthesized - translated protein sequence from 1AGT template

SEQ ID NO: 67 - protein, 250 aa, Artificial Sequence; identical to SEQ ID NO: 63
  Synthesized - translated eGFP protein from 1AGT template (i.e., excluding 17aas from SITS)

SEQ ID NO: 68 - DNA, 804 bp, Artificial Sequence
  Synthesized - 1AGG codon eGFP ORF
atgacagtaatgtataaagtctgtaaagacattaaacacttaggtgaaaccatggagatcaggaagggcgaggagctgttcaccggggtggtgcccatcctggtcgagctggacggcgacgtaaacggccacaagttctcggtgtcgggcgagggcgagggcgatgccacctacggcaagctgaccctgaagttcatctgcaccaccggcaagctgcccgtgccctggcccaccctggtgaccaccctgacctacggcgtgcagtgcttctcgcggtaccccgaccacatgaagcagcacgacttcttcaagtcggccatgcccgaaggctacgtccaggagcggaccatcttcttcaaggacgacggcaactacaagacccgggccgaggtgaagttcgagggcgacaccctggtgaaccggatcgagctgaagggcatcgacttcaaggaggacggcaacatcctggggcacaagctggagtacaactacaactcgcacaacgtctatatcatggccgacaagcagaagaacggcatcaaggtgaacttcaagatccggcacaacatcgaggacggctcggtgcagctggccgaccactaccagcagaacacccccatcggcgacggccccgtgctgctgcccgacaaccactacctgtcgacccagtcggccctgtcgaaagaccccaacgagaagcgggatcacatggtcctgctggagttcgtgaccgccgccgggatcactctgggcatggacgagctatacaaggagcagaagctgatctcggaggaggatctgtaa

SEQ ID NO: 69 - DNA, 3406 bp, Artificial Sequence
  Synthesized - 1AGG codon template DNA sequence
gacgtctaatacgactcactatagggacatcttaagtttattttattttattttattttattttattttattttattttattttattttaattaattttatttaaccatgacagtaatgtataaagtctgtaaagacattaaacacttaggtgaaaccatggagatcaggaagggcgaggagctgttcaccggggtggtgcccatcctggtcgagctggacggcgacgtaaacggccacaagttctcggtgtcgggcgagggcgagggcgatgccacctacggcaagctgaccctgaagttcatctgcaccaccggcaagctgcccgtgccctggcccaccctggtgaccaccctgacctacggcgtgcagtgcttctcgcggtaccccgaccacatgaagcagcacgacttcttcaagtcggccatgcccgaaggctacgtccaggagcggaccatcttcttcaaggacgacggcaactacaagacccgggccgaggtgaagttcgagggcgacaccctggtgaaccggatcgagctgaagggcatcgacttcaaggaggacggcaacatcctggggcacaagctggagtacaactacaactcgcacaacgtctatatcatggccgacaagcagaagaacggcatcaaggtgaacttcaagatccggcacaacatcgaggacggctcggtgcagctggccgaccactaccagcagaacacccccatcggcgacggccccgtgctgctgcccgacaaccactacctgtcgacccagtcggccctgtcgaaagaccccaacgagaagcgggatcacatggtcctgctggagttcgtgaccgccgccgggatcactctgggcatggacgagctatacaaggagcagaagctgatctcggaggaggatctgtaagcggccgccctcctcctcctttcttgttcctttcacgtcgccttctcggttgtagctggcagacgacgagtcttacttttacgtgtacttctctatagatgatgtatgatctctctgcatgcgtgttcgtgcatgtgtccgtgtgttgggtacgcgtggtaccctgcaggaaggaagctgagttggctgctgccaccgctgagcaataactagtaattactagcataaccccttggggcctctaaacgggtcttgagggggttttttgctgaaaggaggacagctgatgattgtcatgcttgccatctgttttcttgcaaggtcagaggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaggatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctagttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 70 - protein, 267 aa, Artificial Sequence
  Synthesized - translated protein sequence from 1AGG template
MTVMYKVCKDIKHLGETMEIRKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEQKLISEEDL

SEQ ID NO: 71 - protein, 250 aa, Artificial Sequence
  Synthesized - translated eGFP protein from 1AGG template (i.e., excluding 17aas from SITS)
MEIRKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEQKLISEEDL

SEQ ID NO: 72 - DNA, 17 bp, Artificial Sequence
  Synthesized - T7 promoter
taatacgactcactata

SEQ ID NO: 73 - DNA, 17 bp, Artificial Sequence
  Synthesized - the reverse complement of the T7 promoter
tatagtgagtcgtatta

SEQ ID NO: 74 - DNA, 84 bp, Artificial Sequence
  Synthesized - Ser (GCT1)
gcaaatgtggccgagtggttaaggcgcctgcctgctaagcaggtgtgatctcacgcgaaggttcgaacccttccgtttgcgcca

SEQ ID NO: 75 - DNA, 84 bp, Artificial Sequence
  Synthesized - Ser (GCT2)
gcaaacgtggccgagtggttaaggcgcctgcctgctaagcaggtgtgatctcacgcgaaggttcgaacccttccgtttgcgcca

SEQ ID NO: 76 - DNA, 84 bp, Artificial Sequence
  Synthesized - Ser (TGA)
gtcaacatacccaagtggttacggggtttgacttgaaatcaaatgcgatctcgcgcgcaggttcgaaccctgctgttgacgcca

SEQ ID NO: 77 - DNA, 84 bp, Artificial Sequence
  Synthesized - Ser (AGA)
gtcaccatacccaagtggttacggggactgactagaaatcagttgcgatctcgcgcgcaggttcgaatcctgctggtgacgcca

SEQ ID NO: 78 - DNA, 84 bp, Artificial Sequence
  Synthesized - Ser (CGA)
gtcaccatacccaagtggttacgggggttgactcgaaatcaactgcgatctcgcgcgcaggttcgaatcctgctggtgacgcca

SEQ ID NO: 79 - DNA, 22 bp, Artificial Sequence
  Synthesized - iLTEagc1
gcaaatgtggccgagtggttaa

SEQ ID NO: 80 - DNA, 21 bp, Artificial Sequence
  Synthesized - iLTEagc2
caaatgtggccgagtggttaa

SEQ ID NO: 81 - DNA, 21 bp, Artificial Sequence
  Synthesized - iLTEagc3
gatctcacgcgaaggttcgaa

SEQ ID NO: 82 - DNA, 23 bp, Artificial Sequence
  Synthesized - iLTEagc4
gtgatctcacgcgaaggttcgaa

SEQ ID NO: 83 - DNA, 22 bp, Artificial Sequence
  Synthesized - iLTEagc5
tgatctcacgcgaaggttcgaa

SEQ ID NO: 84 - DNA, 24 bp, Artificial Sequence
  Synthesized - iLTEagc6
tgtgatctcacgcgaaggttcgaa

SEQ ID NO: 85 - DNA, 25 bp, Artificial Sequence
  Synthesized - iLTEagc7
gtgtgatctcacgcgaaggttcgaa

SEQ ID NO: 86 - DNA, 22 bp, Artificial Sequence
  Synthesized - iLTEagc8
gaaggttcgaacccttccgttt

SEQ ID NO: 87 - DNA, 33 bp, Artificial Sequence
  Synthesized - iLTEagc9
ctaagcaggtgtgatctcacgcgaaggttcgaa

SEQ ID NO: 88 - DNA, 27 bp, Artificial Sequence
  Synthesized - iLTEagc10
aggtgtgatctcacgcgaaggttcgaa

SEQ ID NO: 89 - DNA, 32 bp, Artificial Sequence
  Synthesized - iLTEagc11
taagcaggtgtgatctcacgcgaaggttcgaa

SEQ ID NO: 90 - DNA, 35 bp, Artificial Sequence
  Synthesized - iLTEagc12
tgctaagcaggtgtgatctcacgcgaaggttcgaa

SEQ ID NO: 91 - DNA, 29 bp, Artificial Sequence
  Synthesized - iLTEagc13/14
ctaagcaggtgtgatctcacgcgaaggtt

SEQ ID NO: 92 - DNA, 28 bp, Artificial Sequence
  Synthesized - iLTEagc15
taagcaggtgtgatctcacgcgaaggtt

SEQ ID NO: 93 - DNA, 31 bp, Artificial Sequence
  Synthesized - iLTEagc16
tgctaagcaggtgtgatctcacgcgaaggtt

SEQ ID NO: 94 - DNA, 31 bp, Artificial Sequence
  Synthesized - iLTEagc17
atctcacgcgaaggttcgaacccttccgttt

(19) United States
(12) Patent Application Publication
(10) Pub. No.: US 2022/0002719 A1
(43) Pub. Date: Jan. 6, 2022

(54) OLIGONUCLEOTIDE-MEDIATED SENSE CODON REASSIGNMENT

(71) Applicant: THE UNIVERSITY OF QUEENSLAND, St. Lucia, Queensland (AU)

(72) Inventors: Kirill ALEXANDROV, St. Lucia, Queensland (AU); Sergey MUREEV, St. Lucia, Queensland (AU); Zhenling CUI, St. Lucia, Queensland (AU)

(21) Appl. No.: 17/045,008

(22) PCT Filed: Apr. 2, 2019
(86) PCT No.: PCT/IB2019/000292

§ 371 Date: Oct. 2, 2020

(30) Foreign Application Priority Data
Apr. 3, 2018 (AU) 2018901089

(51) Int. Cl. C12N 15/113; C12P 21/00

(52) U.S. Cl. CPC C12N 15/113; C12P 21/00; C12N 2310/3231; C12N 2310/321; C12N 2310/322; C12N 2310/11

(57) ABSTRACT

Sense codon reassignment to unnatural amino acids (uAAs) represents a powerful approach for introducing novel properties into polypeptides. The main obstacle to this approach is competition between the native isoacceptor tRNA(s) and orthogonal tRNA(s) for the reassigned codon. While several chromatographic and enzymatic procedures for selective deactivation of tRNA isoacceptors in cell-free translation systems exist, they are complex and not scalable. The present invention provides oligonucleotides that hybridise to a tRNA of interest when said tRNA is in a folded state, thereby disrupting the function of the tRNA. The present invention also provides the use of these oligonucleotides in methods for sense codon reassignment and methods for incorporation of uAAs into proteins. The approach described herein represents a new direction in genetic code reassignment with numerous practical applications.

### CROSS-REFERENCE TO RELATED APPLICATIONS

This application is the U.S. national phase of International Application No. PCT/IB2019/000292 filed 2 Apr. 2019, which designated the U.S. and claims priority to AU Patent Application No. 2018901089 filed 3 Apr. 2018, the entire contents of each of which are hereby incorporated by reference.

### FIELD OF THE INVENTION

The present invention is related to protein engineering. In particular, this invention relates to improved methods for producing recombinant proteins comprising one or more non-natural moieties (e.g. unnatural amino acids).

### REFERENCE TO SEQUENCE LISTING SUBMITTED ELECTRONICALLY

The content of the electronically submitted sequence listing (Name: 0117.0788_Sequence_Listing_July_2021.txt; Size: 63.9 kilobytes; and Date of Creation: Jul. 5, 2021) is incorporated herein by reference in its entirety.

### INTRODUCTION

Genetic encoding of unnatural amino acids (uAAs) is a powerful approach for production of polypeptides with novel chemistries and properties (1-3). Polypeptides carrying uAAs are used as molecular probes, biosensors, drug lead candidates and functionalized biologics. While the majority of earlier efforts for uAA incorporation focused on the reassignment of nonsense codons, the idea of exploiting the degeneracy of the genetic code has recently gained popularity (2, 4-6). This approach is attractive due to the large number of redundant sense codons and the lack of competition from the release factors that decrease the efficiency of the nonsense codon reassignment. Yet, the main obstacle for the efficient sense codon reassignment is the competition of native aminoacylated tRNA(s) with the orthogonal tRNA carrying uAA for the chosen sense codon. Hence, effective elimination of the competing endogenous tRNA(s) is critical for the success of this approach.

The concept of sense codon reassignment was initially validated on the example of the rare AGG (Arg) codon in E. coli that was successfully reassigned to various uAAs in vivo (4, 7, 8). These methods required elimination of the competing endogenous Arg-tRNA, achieved through multiple rounds of genetic knockouts and gene complementation. Generally, sense codon reassignment is difficult to perform in vivo due to the abundance of the redundant codons in the endogenous reading frames and the lack of methods for selective elimination of native tRNAs. A radical solution to these problems is construction of organisms with synthetic genomes where both the redundant codons and their cognate native tRNAs are eliminated. Recent publications reported progress towards the goal of complete elimination of redundant codons in E. coli (9). However, deletion of the redundant tRNAs may have broad and unexpected effects on the organism as the former have other functions in addition to genetic decoding (10). Furthermore, it is hard to predict to what extent the freed codons will be miscoded by the remaining native tRNAs. Finally, due to the immense cost of synthetic genome construction, this approach is likely to be limited to bacteria for the foreseeable future. Another avenue for expansion of genome coding capacity is the use of non-canonical codons such as quadruplet codons or codons with unnatural nucleotide structure. While impressive progress has been made in both directions, their broad applicability is still remains to be established (11-13).

An alternative approach that addresses the above mentioned problems is the use of in vitro translation systems, where the levels and identities of the translation reaction components can be easily controlled. Selective depletion of tRNA isoacceptors for amino acids encoded either by mixed codon families or by the families with high wobble restrictions would free the respective codons for decoding with orthogonal tRNAs (o-tRNAs). Suga's group demonstrated the feasibility of this approach for production of peptides containing different uAAs in a fully reconstituted E. coli in vitro translation system (6). Favorable competition of o-tRNAs for the reassigned codons with their endogenous counterparts is achieved by maintaining ultra-high o-tRNA/aaRS ratio. If applied to protein translation, such high tRNA excess may affect the yield and the fidelity of the protein synthesis. In contrast, sense codon reassignment using S30 E. coli lysate (14) would reduce the technical and economic barrier for technology adoption and provide a platform for rapid generation of protein probes for EPR, NMR, cryoEM and single-molecule fluorescence spectroscopy (15, 16). Given that S30 lysate has been adopted to large scale protein manufacturing, the availability of compatible sense codon reassignment methodology would enable industrial production of therapeutic proteins functionalized with novel chemistries (17, 18).

Previously, several approaches for the removal of the entire tRNA pool from the E. coli lysate either by using RNase A-coated beads or ethanolamine-functionalized matrix have been devised (19-23). Such tRNA-depleted lysates could then be supplemented with either native or synthetic tRNAs, thus restoring translation. For example, WO 2016/154675 describes non-specifically depleting substantially all tRNAs for canonical amino acids by binding to ethanolamine sepharose followed by reconstituting a mixture of synthetic tRNAs. One of the main limitations of these approaches is the trade-off between the tRNA depletion efficiency and the remaining translational activity of the lysate (22). The observation that the fully recombinant PURE system contains protein-complexed tRNA fractions large enough to support efficient translation shows that complete and selective tRNA removal is difficult to achieve (22).

Also described in WO 2016/154675 is selective depletion of specific tRNAs (AGG-reading tRNA^{Arg} isoacceptors) from the total tRNA mixture by hybridisation to two complementary DNA oligonucleotides immobilised on beads under thermal denaturing conditions. This method of heat-assisted tRNA fractionation works well, but the chromatographic step is hard to scale. These methods also require reconstitution of the tRNA mixture specifically depleted of certain tRNAs, with a lysate that has been non-specifically depleted for all tRNAs to produce an in vitro cell-free translation system.

Building on the idea of in vitro translation system reconstitution, inactivation of a particular tRNA(s) has been demonstrated (24). The methods involved heating and annealing of the total tRNA fraction with tRNA-specific oligonucleotides, followed by subsequent treatment with RNaseH (24). Heating of the total tRNA fraction was required for RNA:DNA hybridization (24). These methods were shown to deactivate both tRNA^{Asp} and tRNA^{Phe}, which could then be replaced with uAA charged tRNAs. Combination of such tRNA complements with RNase A-treated lysate allowed only partial reassignment of Asp- and Phe-codons to uAAs (24). The partial efficacy of this method is likely due to the incomplete depletion of tRNA^{Asp} and tRNA^{Phe} despite the repeated RNaseH treatment.

Furthermore, both chosen codons were from the split-codon boxes, thus their reassignment would result in loss of the native amino acids as protein building blocks. The sense codon reassignment approach is likely to be most useful when applied to codons of four- and six-fold degenerate amino acids as originally proposed by Kanda for tRNA^{Ser}GCU (19, 24).

Described herein is a generally applicable approach for inactivation of a tRNA of interest in an in vitro translation reaction, which can be applied to a range of tRNAs allowing reassignment of multiple non-identical codons. Synthetic tRNAs carrying the desired unnatural amino acid may be added to restore the translational activity of the protein expression system.

The protein expression systems described herein open up improved ways to create peptides or proteins with novel properties, such as effective drug-antibody conjugates, bioactive peptides with improved bioavailability, synthetic vaccines and novel enzymes with enhanced catalytic activity.

### BRIEF SUMMARY OF THE INVENTION

The present invention provides an oligonucleotide that hybridises to a tRNA of interest when said tRNA is in a folded state, thereby disrupting the function of the tRNA and the use of such oligonucleotides in a method for disrupting the function of a tRNA of interest.

The present invention also provides a method for disrupting function of a tRNA of interest, the method comprising contacting the tRNA in a folded state with an oligonucleotide of the invention. The present invention also provides the following in vitro methods for producing a polypeptide comprising at least one unnatural amino acid:

1) the method comprising incubating:
(a) an mRNA comprising a codon that is recognised by a tRNA of interest;
(b) an oligonucleotide according to any of claims 1-11; and
(c) a tRNA that (i) recognises the same codon as the tRNA of interest, and (ii) is linked to an unnatural amino acid;
under conditions suitable for translation of said mRNA.

2) the method comprising incubating:
(a) an mRNA comprising a codon that is recognised by a tRNA of interest;
(b) an oligonucleotide according to any of claims 1-11;
(c) an orthogonal tRNA that recognises the same codon as the tRNA of interest;
(d) an unnatural amino acid; and
(e) an orthogonal aminoacyl-tRNA synthetase suitable for charging the orthogonal tRNA with the unnatural amino acid;
under conditions suitable for translation of said mRNA.

3) the method comprising in a first step incubating:
(a) an oligonucleotide according to any of claims 1-11; and
(b) one or more tRNAs comprising the tRNA of interest, optionally a full complement of naturally occurring tRNAs comprising the tRNA of interest;
and in a second step incubating:
(c) the mixture resulting from the first step;
(d) an mRNA comprising a codon that is recognised by a tRNA of interest; and either:
(e) a tRNA that (i) recognises the same codon as the tRNA of interest and (ii) is linked to an unnatural amino acid; or
(e) an orthogonal tRNA that recognises the same codon as the tRNA of interest; an unnatural amino acid; and an orthogonal aminoacyl-tRNA synthetase suitable for charging the orthogonal tRNA with the unnatural amino acid;
under conditions suitable for translation of said mRNA.

The present invention further provides compositions and kits for use in the methods of the present invention. For example, the present invention provides a composition or kit comprising: (a) an oligonucleotide of the invention; and (b) one or more of: (i) a tRNA that (a) recognises the same codon as the tRNA of interest, and (b) is linked to an unnatural amino acid; (ii) one or more tRNAs comprising the tRNA of interest; and/or (iii) one or more translation reagents. The present invention also provides a composition or kit comprising: (a) an oligonucleotide of the invention; and (b) one or more of: (i) an orthogonal tRNA that recognises the same codon as the tRNA of interest; (ii) an unnatural amino acid suitable for coupling to the tRNA; (iii) an orthogonal aminoacyl-tRNA synthetase suitable for charging the orthogonal tRNA with the unnatural amino acid; (iv) one or more tRNAs, comprising the tRNA of interest; and/or (v) one or more translation reagents.

The present invention also provides a vector suitable for expressing an oligonucleotide of the invention and a cell comprising said vector and/or an oligonucleotide of the invention. The present invention also provides a lysate prepared from said cells comprising oligonucleotides of the invention and the use of such a lysate in an in vitro method for producing a polypeptide comprising at least one unnatural amino acid, the method comprising incubating together: (a) the lysate; (b) an mRNA comprising a codon that is recognised by the tRNA of interest; and (c) a tRNA that (i) recognises the same codon as the tRNA of interest, and (ii) is linked to an unnatural amino acid; under conditions suitable for translation of said mRNA.

The present invention also provides a polypeptide comprising one or more unnatural amino acids produced according to any of the methods of the invention.

### DESCRIPTION OF THE FIGURES

FIG. 1. Design and analysis of oligonucleotides targeting E. coli tRNA^{Ser}GCU. (A) Schematic representation of the antisense oligonucleotides targeting anticodon/variable loop (M1-M5) or D-arm/anticodon regions (M6-M8) of tRNA^{Ser}GCU. (B) Table of the affinities of the antisense oligonucleotides for tRNA^{Ser}GCU as measured by MST. The measurements were performed by titrating 10 nM solution of fluorescently labelled antisense oligonucleotide with increasing concentrations of tRNA^{Ser}GCU. The K_{d} values were calculated using a nonlinear fit equation of the law of mass action (FIG. 7). ND—no detectable binding. The error bars represent standard deviations of three experiments. (C) Denaturing PAGE analysis of 5 pmole of Cy3-labelled antisense oligonucleotide mixed with 25 pmole of the indicated tRNAs. The positions of the oligonucleotides were visualized by the fluorescent scanning of the gel. (D) Interaction analysis between oligonucleotide M5-1 and Cy3-labelled tRNA^{Ser}GCU. The affinity was measured as described in (B) but using a 5 nM solution of Cy3-labelled tRNA titrated with the increasing concentrations of M5-1. The error bars represent standard deviations of three experiments.

FIG. 2. Testing the ability of the antisense oligonucleotides to selectively inactivate tRNA isoacceptors in E. coli cell-free translation system. (A) A schematic representation of the eGFP fluorescent reporter construct for evaluation of tRNA^{Ser}GCU levels in cell-free system. The wt eGFP-coding ORF designed with biased Ser-codons is prefaced by Species Independent Translation Initiation Sequence (SITS) that enables translation in both pro- and eukaryotic cell-free systems. (B) Translation activity of the 2AGC-codon template in E. coli lysate pre-treated with oligonucleotides in the presence or absence of synthetic tRNA^{Gly}GCU. The MS-sequence analogs M5-1,-2,-3 contain various percentages of modified residues (100%, 75% and 46%, respectively). The concentration of the antisense oligonucleotides for lysate treatment is indicated while tRNA^{Gly}GCU is added to final concentration of 20 μM in the translation reaction. The relative translation activity was calculated as the percentage of activity of the parental lysate. The error bars represent standard deviations of three experiments. (C) The schematic representation of the methylated RNA oligonucleotides targeting four regions of tRNA^{Arg}CCU (R1, 2, 3, 4). Each oligonucleotide is represented by a black line with thickness proportionate to their inhibition efficiency as shown in FIG. 2D. (D) Translational activity of eGFP-coding template harbouring single AGG (Arg) codon (Table 2) in E. coli lysate treated with 10 μM oligonucleotides R1-R4. The expression activity is represented as described in (B). The error bars represent standard deviations derived from the mean value of the relative activity (n=3).

FIG. 3. Truncation analysis of M5-1 oligonucleotides. (A) Schematic representation of the M5-1 truncations. The black line shows the region of tRNA^{Ser}GCU targeted by M5-1 oligonucleotide. M5T1 and M5T2 are truncated from 5′-end while M5T3 and M5T4 are truncated from 3′-end (indicated in the figure by the arrows). (B) Translational activity of 2AGC-codon eGFP-coding template in antisense oligo-treated E. coli lysate. The used concentrations of the oligonucleotides are coded by increasing shading of the graphs. Each translation reaction was repeated twice. (C) Proposed mechanism of antisense oligonucleotide: tRNA interaction. The crystal structure of a tRNA^{Sec} (PDB: 3W3S) was used to represent the E. coli tRNA^{Ser}GCU. The loop regions are coloured in grey while the stems are shown in colour. The nucleotides in the variable loop and the anticodon loop that are expected to be solution-exposed are marked. The antisense oligonucleotide is shown as an unstructured single-stranded sequence. The initial binding (Step 1) of the methylated antisense oligonucleotide to tRNA^{Ser}GCU is proposed to initiate at the four consecutive adenosines of the variable loop facilitating duplex propagation (Step 2).

FIG. 4. Reassignment of AGT codon (Ser) in the open reading frame of eGFP to n-propargyl-L-lysine and p-azido-L-phenylalanine. (A) Structures of n-propargyl-L-lysine (Prk) and p-azido-L-phenylalanine (AzF). (B) Reassignment of AGT codon to Prk using eGFP-coding template with single AGT. The bar chart shows relative fluorescence of the M5-1 oligonucleotide treated lysate in the presence or absence of 10 μM of tRNA^{Pyl}ACU and PylRS as well as PrK at 1 mM. To confirm AGT codon suppression specificity tRNA^{Gly}GCU was used as a positive control. eGFP fluorescence produced in parental lysate was used to calculate the relative translation activity. Each translation reaction was repeated at least twice. (C) LC-MS/MS analysis of Prk incorporated via AGT codon in eGFP using tRNA^{Pyl}ACU, PylRS and PrK. The mass of the detected peptide of 634.33 Da is close to the calculated mass for PrK-modified peptide (634.26 Da). (D) Reassignment of AGT codon to AzF using eGFP template from (B). The translational activity of the M5-1 oligonucleotide treated lysate are measured in the presence or absence of 10 μM of tRNA^{AzF}ACU, AzFRS and 1 mM AzF. Each translation reaction was repeated at least twice. (E) LC-MS/MS analysis of AzF incorporation at AGT codon of the eGFP ORF using tRNA^{AzF}ACU/AzFRS/AzF. The detected mass of 623.31 Da is close to the predicted mass of 623.26 Da of AzF.

FIG. 5. 2′OMe antisense oligonucleotide mediated inactivation of tRNA^{Ser}GCU in the eukaryotic cell-free translation system based on L. tarentolae-extract (LTE). (A) Monitoring of eGFP production in the total tRNA-depleted LTE translation system upon its supplementation with the indicated amount of the total L. tarentolae tRNA. Each translation reaction was repeated at least twice. (B) The schematic representation of the 2′OMe antisense oligos to L. tarentolae tRNA^{Ser}GCU (L1-L6). (C) Inactivation of tRNA^{Ser}GCU by antisense oligonucleotides in the context of total L. tarentolae tRNA. The resulting tRNA mixture and all tRNA-depleted LTE was used for reconstitution of the L. tarentolae in vitro translation system programmed by 2AGC-codon eGFP-coding template. Antisense oligonucleotide was added to 10 μM final concentration. The error bars represent standard deviations of two experiments. (D) 2′OMe antisense oligonucleotide mediated inactivation of L. tarentolae tRNA^{Ser}GCU in LTE lysate. The LTE lysate was incubated with L4 oligo at 37° C. for 5 min and then used for translation of 2AGC-codon eGFP-coding template. Antisense oligonucleotide was added to 15 μM final concentration. In all experiments tRNA^{Gly}GCU-suppressor was added to 20 μM final concentration. Each translation reaction was repeated at least twice.

FIG. 6. Application of RNA transcripts-mediated suppression in E. coli cell-free system. (A) Analysis of translational activity of the F1-treated E. coli lysate. The E. coli lysate was incubated with F1 oligonucleotides at 37° C. for 5 min and then used for assembly the translation reaction priming with eGFP template containing 2AGC codons. In the control experiment tRNA^{Gly}GCU was added to 20 μM final concentration to rescue the AGC translation. (B) Analysis of translational activity of the reconstituted in vitro translation system composed of total tRNA pre-incubated with antisense oligonucleotides (targeting the indicated tRNAs) and depleted E. coli cell lysate. The reactions were primed with eGFP templates with different codon compositions. For tRNA^{Arg}CCG testing, the template contains 6 CGG and 1 AGG codon coding Arg. For tRNA^{Ser}GGA/UGA as well as the tRNA^{iMet}CUA, the 2 TCC codon template was employed with 2 TCC codons and 1 start codon. In the control experiment their respective synthetic tRNAs were added to 20 μM final concentration. Each translation reaction was repeated twice.

FIG. 7. MST analysis of the interaction of the antisense oligonucleotides and synthetic tRNA^{Ser}GCU. The measurements were performed by titrating 10 nM Cy3-labelled antisense oligonucleotide (M1-M8) with increasing concentrations of tRNA^{Ser}GCU. The K_{d} values were calculated using nonlinear fit equation of the law of mass action.

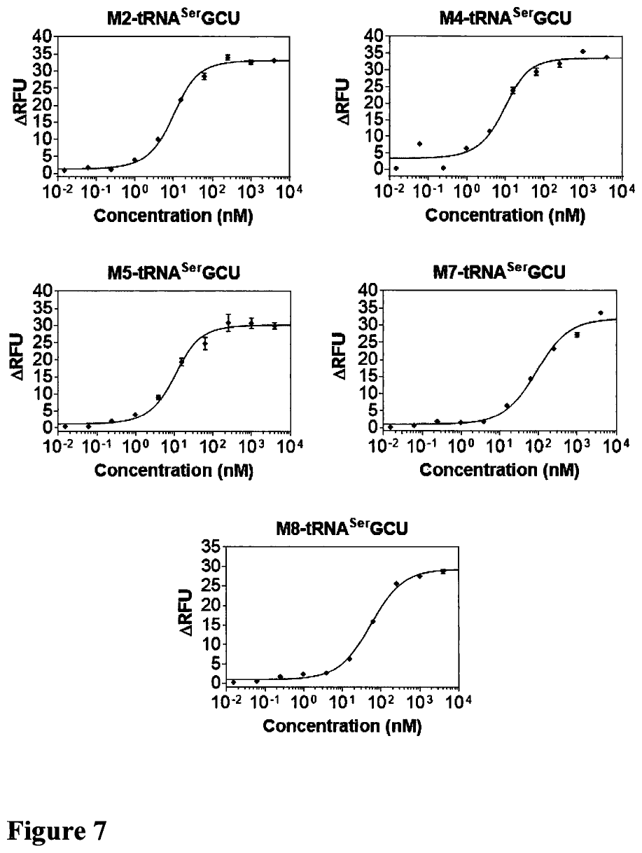

FIG. 8. Expression levels of eGFP produced by pLTE vector encoding a 2AGC-codon template in E. coli lysate pre-treated with oligonucleotides in the absence or presence of synthetic tRNA^{Gly}GCU. (A) Translation activity of E. coli lysate pre-treated by M1-M8 oligonucleotides. The lysate was treated with 30 μM of antisense oligos resulting in final concentration of 10 μM in the translation reaction. The tRNA^{Gly}GCU was added to the translation reaction to 20 μM final concentration. The relative translation activity was calculated by comparing the translation efficiency to that of the untreated lysate. The increase in protein yield upon supplementation with synthetic tRNA^{Gly}GCU is indicated above the bars representing each experiment. Each translation reaction was repeated twice. (B) Translational activity of S30 lysate treated with 5 μM M4 or 2 μM M5 oligos. Each translation reaction was repeated twice. Partially and fully modified oligonucleotides M4 and M5 targeting AV-region caused the highest translation inhibition while still affording its recovery with synthetic tRNA. Reducing the concentration of M5 to 2 μM allowed efficient translation inhibition to ˜10% while tRNA^{Gly}GCU-mediated recovery reached ˜55%. The recovery of the expression was not complete possibly due to the quenching of the eGFP fluorescence by the Cy3 fluorophore attached to the oligonucleotide.

FIG. 9. Superposition of tRNA^{Ser} with tRNA^{Leu} or tRNA^{Sec} structures. The PDB structures 1Ser (tRNA^{Ser}), 4V8C (chain CB and DB, tRNA^{Leu}) and 3W3S (tRNA^{Sec}) coloured in orange, green and blue, respectively, were superimposed using PyMOL software.

FIG. 10. Alignment of five Lt tRNA^{Ser} isoacceptors with the antisense oligonucleotides designed using OligoWalk. The stem regions of the tRNA are marked with various colours. Light blue indicates the acceptor stem; orange shows the D-stem, green colours the anticodon stem, yellow is the variable stem while the magenta is the T stem. For targeting Lt tRNA^{Ser}GCU, we set a range of antisense oligo lengths from 18 till 35 where the OligoWalk does not generate any hits due to the long length and unfavourable thermodynamics to form the oligo-tRNA complex. A total of 17 antisense oligos designed using OligoWalk were marked as iLTEagc1 to iLTEagc17 with more than 0.5 probability of being efficient siRNA predicted by the software. We ranked all the oligos according to their probability of being efficient siRNA. They clearly formed two clusters, one targeting the 5′ of the tRNA and the other targeting tRNA from variable loop or anticodon stem till T loop. We chose the best hit of each cluster, iLTEagc1 (L5) and iLTEagc3 (L6), with predicted 0.86 and 0.84 probability of being efficient interfering RNA, respectively.

### DETAILED DESCRIPTION

The present Inventors have demonstrated that appropriately-designed antisense oligonucleotides are able to bind to and sequester a specific tRNA isoacceptor of interest in situ in a folded state in a cell extract, a cell lysate, and/or a cell-free translation system, to free the corresponding codon for utilisation by an unnatural amino acid (uAA). Active depletion of the tRNA of interest prior to codon reassignment is thus unnecessary. The cell extract, cell lysate, and/or cell-free translation system treated with such an oligonucleotide is supplemented with an orthogonal tRNA that recognises the same codon as the sequestered/bound tRNA of interest and is pre- or co-translationally charged with the desired unnatural amino acid (uAA). The orthogonal tRNA delivers the uAA site-specifically to the newly created orthogonal codon. The freed codon is used for uAA functionality while its synonymous codons from another codon box still code for the canonical amino acid without sacrificing the amino acid library for protein production.

In addition, the antisense oligonucleotides are able to bind to and sequester a specific tRNA of interest in a folded state in native total tRNA mixture; i.e., a purified mixture of the total native tRNAs from a cell extract/cell lysate. All of the tRNA species (i.e., the total tRNA) in the cell extract/cell lysate may be purified as described in WO 2016/154675, for example by phenol extraction. The oligonucleotide is added to the total tRNA mixture and binds/sequesters a specific tRNA of interest in a folded state to free the corresponding codon for utilisation by a uAA. Selective depletion of the tRNA of interest prior to codon reassignment is thus unnecessary. A cell free protein translation reaction may be reconstituted from the total tRNA depleted cell lysate/extract and the oligonucleotide-treated total tRNA mixture. Such a cell free protein translation reaction may be used for sense codon reassignment when supplemented with an orthogonal tRNA that recognises the same codon as the tRNA of interest, pre- or co-translationally charged with the desired uAA. The present invention is described in more detail below.

### Oligonucleotides

The present invention provides an oligonucleotide that hybridises to a tRNA of interest when said tRNA is in a folded state, thereby disrupting the function of the tRNA. In the context of this invention, the term “oligonucleotide” refers to an oligomer of nucleotide or nucleoside monomers comprising bases, sugars and inter-sugar backbone linkages. The term “oligonucleotide” also includes oligomers comprising non-naturally occurring monomers, or portions thereof, which function similarly. Such modified oligonucleotides are often preferred over native forms because of properties such as, for example, enhanced stability of the oligo-tRNA hybrid and reduced degradation, for example, in the presence of nucleases.

The structural features of oligonucleotides of the invention are described below. Discussion of the functional features of the oligonucleotides is provided in the context of description of their interaction with the tRNA of interest.

Oligonucleotides are generally classified as deoxyribo-oligonucleotides or ribo-oligonucleotides, which are oligomers of DNA or RNA molecules. The oligonucleotides of the present invention may be double-stranded or single-stranded DNA or RNA, preferably RNA. Such RNA oligonucleotides may be substantially single-stranded or may comprise regions having secondary and tertiary structure.

The oligonucleotides of the present invention may have any sequence. Further preferred characteristics of the oligonucleotides are defined herein. The oligonucleotides of the invention may comprise or consist of any or all of the natural nucleotides, including adenosine, guanosine, thymidine, uridine, cytidine and inosine. The oligonucleotides of the invention may be unmodified. The oligonucleotides of the invention may comprise no modified nucleotides. Alternatively, the oligonucleotides of the invention may comprise modified nucleotides. Thus, the oligonucleotide of the invention may be modified by the substitution of at least one nucleotide with at least one modified nucleotide, ideally so that the stability of the oligonucleotide is enhanced as compared to a corresponding unmodified oligonucleotide. The modified nucleotide may, for instance, be a sugar-modified nucleotide or a nucleobase-modified nucleotide. Suitable modified nucleotides include 2′-methyl, 2′-fluoro, 2′-amino or 2′-thioester modified nucleosides, preferably 2′-methyl or 2′-fluoro modified nucleotides. The modified nucleotide may be selected from 2′-O-methylcytidine, 2′-O-methyladenosine, 2′-O-methylguanosine, 2′-O-methyluridine, 2′-O-methylinosine, 2-fluorocytidine, 2-fluoroadenosine 2-fluoroguanosine, 2-fluorouridine, 2-fluoroinosine, locked nucleic acids (LNAs), and phosphorodiamidate morpholino oligomers (PMOs; which comprise DNA bases attached to a backbone of methylenemorpholine rings linked through phosphorodiamidate groups), and nucleotides linked by phosphorothioate. The oligonucleotides of the invention may comprise at least 40%, 50%, 60%, 70%, 75%, 80%, 85%, 90%, 95%, 98%, 99% or 100% modified nucleotides or between 40%-100%, 50%-100%, 60%-100%, 70%-100%, 80%-100%, 90%-100% or 95%-100% modified nucleotides. The oligonucleotides of the invention may comprise at least 75% modified nucleotides, preferably 75%-100% modified nucleotides. In preferred instances, the oligonucleotides of the invention comprise substantially 100% modified nucleotides. In preferred instances, the modified nucleotides are 2′-O-methylated.

The oligonucleotides of the present invention may comprise any modifications. The oligonucleotides of the invention may comprise any modifications that could be introduced into oligonucleotides at the time of synthesis. For example, the oligonucleotides may comprise modifications including phosphorylation, reactive groups with/without linkers (such as NHS ester, amino modifier with variable linkers, alkynes, biotin, etc.), fluorophores, spacers, modified bases, and/or phosphorothioate bonds. Further preferred characteristics of the oligonucleotides are described herein.

In some instances the oligonucleotides of the present invention comprise a fluorophore. For example, the oligonucleotides may be covalently bonded to (i.e., conjugated to) a fluorophore. The fluorophore may be conjugated to any nucleotide in the oligonucleotide, the fluorophore may be conjugated to the 5′- or 3′-end of the oligonucleotide, preferably to the 5′-end. The skilled person would be capable of selecting a suitable fluorophore, for example that enables detection of the oligonucleotide by measuring the fluorescence at a suitable wavelength. The fluorophore may be any suitable fluorophore. For example, the fluorophore may be a fluorescent dye, such as fluorescein, FITC, BODIPY, Cy3, Cy5, Cy7, Cy2, TRITC, Rhodamine, Texas Red, Allophycocyanin, Pacific Blue, Pacific Orange, Lucifer yellow; a quantum dot or a fluorescent protein, such as GFP, CFP, YFP, TFP, Venus, Orange, Kate. In some instances the fluorophore will have a fluorescence emission spectrum that does not overlap with that of eGFP. Preferably, the fluorophore is Cy3. In some instances the oligonucleotides of the present invention do not comprise a fluorophore.

The oligonucleotides of the present invention may be between 10-80, 15-70, 20-60, 30-50 or 40-50 nucleotides in length, optionally at least 10, 15, 20, 30, 40, 50 nucleotides in length. Preferably the oligonucleotides may be between 15-60 nucleotides in length. Nucleotides as used herein encompasses both natural nucleotides (comprising adenine, guanine, cytosine, thymine, uracil or inosine) and modified nucleotides (including 2′-methyl, 2′-fluoro modified or LNAs). The oligonucleotides may form secondary structure, for example, through internal Watson-Crick base pairing or wobble base pairing.

Watson-Crick base pairing, also known as complementary base pairing, describes the formation of two hydrogen bonds between adenine and thymine (or uracil in RNA) (a A-T or A-U base pair) and the formation of three hydrogen bonds between guanine and cytosine (a G-C base pair). Non-Watson-Crick base pairing, also known as wobble base pairing, does not follow Watson-Crick base pair rules. The four main wobble base pairs are guanine-uracil (G-U), hypoxanthine-uracil (I-U), hypoxanthine-adenine (I-A), and hypoxanthine-cytosine (I-C). The nucleobase hypoxanthine corresponds to the nucleoside inosine. Both intermolecular (between at least two polynucleotides) and intramolecular base pairing may occur. Intramolecular base pairing within single-stranded RNA molecules allows for the formation of double-stranded helices, complex secondary structure and three-dimensional tertiary structures.

In some instances, the oligonucleotides of the invention may comprise a sequence selected from any one of SEQ ID NOs 1-31, or a variant thereof. In some instances, the oligonucleotides of the invention may comprise a sequence selected from any one of SEQ ID NOs 1-5, 9-16 or 17-31, or a variant thereof. In some instances, the oligonucleotides of the invention may comprise a sequence selected from any one of SEQ ID NOs 2-5, 9-16 or 17-26, or a variant thereof. In some instances, the oligonucleotides of the invention may comprise a sequence selected from any one of SEQ ID NOs 2, 4, 5, 9, 10, 11, 14, 16, 18, 24, or a variant thereof. In some instances, the oligonucleotides of the invention may comprise a sequence selected from any one of SEQ ID NOs SEQ ID NOs 5, 9, 14, 16, 18, 24, or a variant thereof. In some preferred instances, the oligonucleotides of the invention may comprise a sequence selected from any one of SEQ ID NOs 9, 18, 24, or a variant thereof. In some instances, the oligonucleotides of the invention may comprise a sequence selected from SEQ ID NO: 9, or a variant thereof. In some instances, the oligonucleotide may hybridise to E. coli tRNA^{Ser}GCU and may comprise a sequence selected from any one of SEQ ID NOs 1-16, preferably a sequence selected from any one of SEQ ID NOs 5, 9, 14 or 16, or a variant thereof. In some instances, the oligonucleotide may hybridise to E. coli tRNA^{Arg}CCU and may comprise a sequence selected from any one of SEQ ID NOs 17-20, or a variant thereof. In some instances, the oligonucleotide may hybridise to L. tarentolae tRNA^{Ser}GCU and may comprise a sequence selected from any one of SEQ ID NOs 21-26, or a variant thereof. In some instances, the oligonucleotide may hybridise to E. coli tRNA^{Ser}GCU and may comprise the sequence of SEQ ID NO: 27, or a variant thereof. In some instances, the oligonucleotide may hybridise to E. coli tRNA^{Ser}GGA and may comprise the sequence of SEQ ID NO: 28, or a variant thereof. In some instances, the oligonucleotide may hybridise to E. coli tRNA^{Ser}UGA and may comprise the sequence of SEQ ID NO: 29, or a variant thereof. In some instances, the oligonucleotide may hybridise to E. coli tRNA^{Arg}CCG and may comprise the sequence of SEQ ID NO: 30, or a variant thereof. In some instances, the oligonucleotide may hybridise to tRNA^{iMet}CAU and may comprise the sequence of SEQ ID NO: 31, or a variant thereof.

The term “variant” includes substitution of any deoxyribonucleotide for the corresponding ribonucleotide (for example, a thymidine (T) nucleotide in a DNA sequence may be a uridine (U) nucleotide in a variant); substitution of any ribonucleotide for the corresponding deoxyribonucleotide; substitution of any ribonucleotide or deoxyribonucleotide for the corresponding modified nucleotide (as defined herein); substitution of any ribonucleotide or deoxyribonucleotide for the corresponding 2′-fluoro ribonucleotide; and/or substitution of any ribonucleotide or deoxyribonucleotide for the corresponding 2′-O-methyl ribonucleotide. Where a sequence is a DNA sequence, the variant may be the corresponding RNA sequence or the corresponding 2′-O-methyl-RNA sequence. The term “variant” includes addition or removal of 5′ and 3′ labels and modifiers, for example, the addition or removal of a 3′ amino modifier or a 5′ Cy3 label. The term “variant” includes the deletion of 1, 2, 3, 4, 5, 6, 7, 8, 9, 10 nucleotides from the 5′ and/or 3′ end of the sequence, preferably the deletion of up to 5 nucleotides from the 5′ and/or 3′ end, most preferably the deletion of 1-2 nucleotides from the 5′ and/or 3′ end, preferably the 3′ end. The term variant also includes sequences having at least 60%, 70%, 80%, 90%, 95%, 96%, 97%, 98% or at least 99% sequence identity to a sequence selected from any one of SEQ ID NOs 1-31, preferably at least 95% sequence identity and most preferably at least 98% sequence identity to a sequence selected from any one of SEQ ID NOs 1-31. In preferred instances, the term variant includes sequences having at least 60%, 70%, 80%, 90%, 95%, 96%, 97%, 98% or at least 99% sequence identity to a sequence selected from any one of SEQ ID NOs 2, 4, 5, 7, 8, 9, 10, 11, 14, 16, 18 or 24; preferably to a sequence selected from any one of SEQ ID NOs 5, 9, 18 or 24. In the most preferred instances, the term variant includes sequences having at least 95% sequence identity and most preferably at least 98% sequence identity to a sequence selected from any one of SEQ ID NOs 2, 4, 5, 7, 8, 9, 10, 11, 14, 16, 18 or 24; preferably to a sequence selected from any one of SEQ ID NOs 5, 9, 18 or 24.

Sequence identity between two sequences is preferably determined using pairwise global sequence alignment, wherein the alignment is calculated over the length of the sequence of described herein. Sequence identity may preferably be calculated using the Needleman-Wunsch alignment algorithm (for example as implemented through the online server EMBOSS Needle (EMBOSS: the European Molecular Biology Open Software Suite. (2000) Trends in genetics. 16(6):276-7) and applying the following parameters: Matrix: DNAfull; Gap open penalty: 10.00; Gap extension penalty: 0.5; End Gap penalty: false; End Gap open penalty: 10.00; End Gap extension penalty: 0.5.

The present invention further provides a vector suitable for expressing an oligonucleotide of the invention. The vector may be suitable for prokaryotic or eukaryotic expression of the oligonucleotide. The vector may be a plasmid. The vector may be integrated into the genome of a host cell. The vector may comprise an inducible promoter, optionally a chemically-regulated or physically-regulated promoter. The present invention also provides a cell comprising the vector of the invention or a cell expressing the oligonucleotides of the invention. The cell may be eukaryotic or prokaryotic. The cell may be a yeast cell, preferably Saccharomyces cerevisiae or Schizosaccharomyces pombe, a bacterial cell, preferably Escherichia coli, or a trypanosome, preferably Leishmania tarentolae. Preferably, the cell is an Escherichia coli or Leishmania tarentolae cell.

In some instances, the oligonucleotides are RNA oligonucleotides and are synthesised from a DNA template using an RNA polymerase such as T7 RNA polymerase. Thus, the present invention provides a DNA polynucleotide comprising a promoter, such as a T7 promoter, operably linked to a DNA nucleotide encoding an oligonucleotide of the invention (i.e., the DNA nucleotide comprises the corresponding DNA sequence of an oligonucleotide of the invention). Also provided is a double-stranded DNA molecule comprising a coding strand comprising a promoter, such as a T7 promoter, operably linked to the corresponding DNA sequence of an oligonucleotide of the invention and optionally a template strand comprising the reverse complement of the promoter, such as the T7 promoter, operably linked to the DNA reverse complement of an oligonucleotide of the invention. The double-stranded DNA molecule or the DNA polynucleotide is suitable for producing an RNA oligonucleotide of the invention by transcription.

The promoter may be any promoter that is recognised by a DNA-dependent RNA polymerase, i.e., an RNA polymerase that is suitable for transcribing a DNA sequence to produce an RNA oligonucleotide. For example, an SP6 promoter used with an SP6 RNA polymerase, a T3 promoter used with a T3 polymerase, or a T7 promoter used with a T7 polymerase. In preferred instances, the promoter is a T7 promoter and the DNA polynucleotide may be transcribed with a T7 polymerase. The T7 promoter may have the sequence 5′-TAATACGACTCACTATA-3′ (SEQ ID NO: 72) and accordingly the reverse complement of the T7 promoter may have the sequence 5′-TATAGTGAGTCGTATTA-3′ (SEQ ID NO: 73). In some instances, an extra “G” is added in front of the T7 promoter in the DNA polynucleotide. This increases the Tm of the “forward” oligonucleotides for PCR production of the DNA-template. In some instances, where the RNA oligonucleotide of the invention starts with either “T” or “A”, an extra “G” is added to the start of the corresponding DNA sequence of the oligonucleotide of the invention in the DNA polynucleotide. Where the RNA oligonucleotide of the invention starts with either “G” or “C”, no extra “G” is added.

The double-stranded DNA molecule, preferably the template stand, may provide a substrate (i.e., a target) for the DNA-dependent RNA polymerase, preferably T7 RNA polymerase. Transcription of a double-stranded DNA molecule of the invention, for example by T7 RNA polymerase, produces an RNA oligonucleotide according to the present invention. Other promoters and RNA polymerases are suitable for the purpose of faithfully transcribing the template, such as SP6 promoter and SP6 RNA polymerase, to produce an RNA oligonucleotide according to the present invention.

tRNAs of Interest

The oligonucleotides of the present invention hybridise to tRNAs of interest. The tRNA of interest may be selected from the full complement of endogenous or natural tRNAs. As used herein, “the full complement of endogenous or natural tRNAs” refers to all of the naturally occurring tRNAs found within an organism of interest, i.e., the organism providing the necessary components for protein translation, or the organism providing the cells from which the cell extract, cell lysate or in vitro protein translation system is generated. For example, the tRNA of interest may be an E. coli tRNA and the full complement of endogenous or natural tRNAs would comprise all of the naturally occurring tRNAs found in E. coli. The number of tRNAs and/or the structure of the tRNAs may differ between different organisms. The tRNA of interest may be an endogenous or natural tRNA for any organism, such as a eukaryotic or prokaryotic organism. The tRNA of interest may be a prokaryotic tRNA or a eukaryotic tRNA. The tRNA of interest may be an endogenous or natural human, mammalian, yeast, bacterial or trypanosome tRNA, preferably an endogenous or natural bacterial or trypanosome tRNA. In preferred embodiments, the tRNA of interest may be an endogenous or natural Saccharomyces cerevisiae, Schizosaccharomyces pombe, Escherichia coli, or Leishmania tarentolae tRNA, preferably an endogenous or natural Escherichia coli or Leishmania tarentolae tRNA.

The tRNA of interest may recognise any codon. Preferably, the tRNA of interest recognises a codon that encodes a three-fold, four-fold or six-fold degenerate amino acid (i.e., a codon belonging to a three-fold, four-fold or six-fold degenerate codon family/group), such as those codons encoding isoleucine, alanine, glycine, proline, threonine, valine, arginine, leucine or serine, or the start codon. Preferably, the tRNA of interest recognises (i) a codon encoding a six-fold degenerate amino acid such as a codon encoding arginine, leucine or serine; (ii) the initiator methionine codon, or (iii) a codon encoding a four-fold degenerate amino acid such as a codon encoding alanine, proline, glycine, valine or threonine. Thus, the tRNA of interest may be selected from tRNA^{Arg}, tRNA^{Leu}, tRNA^{Ser}, tRNA^{iMet}, tRNA^{Ala}, tRNA^{Pro}, tRNA^{Gly}, tRNA^{Val} or tRNA^{Thr}, optionally the tRNA of interest may be selected from tRNA^{Ser}_{GCU}, tRNA^{Ser}_{GGA}, tRNA^{Ser}_{UGA}, tRNA^{Ser}_{CGA}, tRNA^{Arg}_{CCU}, tRNA^{Arg}_{UCU}, tRNA^{Arg}_{CCG}, initiator tRNA^{iMet}, tRNA^{Leu}_{CAA}, tRNA^{Leu}_{CAG} and tRNA^{Leu}_{GAG}. Most preferably, the tRNA of interest may be selected from tRNA^{Ser}_{GCU}, tRNA^{Arg}_{CCU}, RNA^{Ser}_{GGA}, tRNA^{Ser}_{UGA}, tRNA^{Arg}_{CCG}, initiator tRNA^{iMet}. The codons decoded by these tRNAs include AGU/C, AGG, UCC/U, UCA/U/C, CGG, and AUG (initiator), respectively.

The oligonucleotides of the present invention hybridise to (i.e. bind to) a tRNA of interest. The oligonucleotides of the present invention hybridise to a tRNA of interest when said tRNA is in a folded state. Hybridisation describes the process of a portion of a DNA or RNA oligonucleotide binding or annealing to a complementary or substantially complementary portion of a second DNA or RNA oligonucleotide to produce DNA-DNA, DNA-RNA or RNA-RNA hybrids. Hybridisation may occur through Watson-Crick or wobble base pairing, preferably through Watson-Crick base pairing. In order for two oligonucleotide sequences to hybridise they must be complementary or substantially complementary, although a certain number of mismatches are tolerated. The oligonucleotides of the present invention are complementary or substantially complementary to a sequence within the tRNA of interest.

The term “complementary” is used to describe two polynucleotide sequences that are able to base pair, either through Watson-Crick or wobble base pairing, at nucleotide positions across the length of both sequences. The term “fully complementary” (i.e., 100% complementarity) is used to describe two polynucleotide sequences which base pair, either through Watson-Crick or wobble base pairing, at every nucleotide position across the whole length of the shorter of the two sequences (i.e., there are no mismatches). The term “substantially complementary” is used to describe two sequences which comprise less than 100% complementarity (over the length of the shorter sequence). Such fully complementary, complementary and substantially complementary sequences may hybridise or bind over any temperature or pH range. In particular, such fully complementary, complementary and substantially complementary sequences are capable of hybridising at temperatures between 18-37° C. and at physiological pH (between 5-9). Substantially complementary sequence may have a number of mismatches, but still hybridise or bind to each other. Two substantially complementary sequences may comprise 1, 2, 3, 4, 5, 6, 7, 8, 9, 10, 11, 12, 13, 14, 15, 16, 17, 18, 19 or 20 mismatches, optionally between 1-20, 2-10, 5-15, 10-20 or 8-12 mismatches, for every 20, 50 or 100 nucleotides in length of the shorter of the two sequences, preferably between 1-10 mismatches for every 20 nucleotides in length of the shorter sequence. Two substantially complementary sequences may comprise at least 1%, 2%, 3%, 4%, 5%, 10%, 15% or 20% mismatches, optionally between 1-50%, 1-40%, 1-30%, 1-20%, 1-10%, 5-10% or 1-5% mismatches, preferably between 1%-25% mismatches (as a percentage of the number of nucleotides in the shorter sequence). A mismatch may be defined as two nucleotides that do not base-pair either through Watson-Crick or wobble base pairing.

The oligonucleotides of the present invention hybridise to a tRNA of interest when said tRNA is in a folded state. It should not be understood that the oligonucleotides of the invention may only bind to a tRNA in a folded state, but rather that they are capable of binding to a tRNA in a folded state. The oligonucleotides of the present invention may also hybridise to a tRNA of interest when said tRNA is in an unfolded state or to a tRNA that is in equilibrium between folded and unfolded states. The folded state of a tRNA of interest, as described herein, may be understood by reference to the primary, secondary and tertiary structure of tRNAs. The primary structure of a tRNA is its ribonucleotide sequence. The secondary structure of a tRNA describes the intramolecular base pairing (or hydrogen bonding) pattern and is typically visualised using the cloverleaf representation. The tertiary structure of a tRNA describes its three-dimensional arrangement in space. All tRNAs adopt a compact L-shaped three-dimensional structure that allows for binding at the P and A sites of the ribosome. The folded state of a tRNA is its tertiary structure, the compact L-shape. A tRNA typically comprises:
(i) a 5′-terminal phosphate group;
(ii) an acceptor stem consisting of a 7-9 base pair (bp) stem formed through base pairing of the 5′-terminal nucleotide with the nucleotide preceding the 3′-terminal CCA tail;
(iii) the 3′-CCA tail, which is recognised by aminoacyl tRNA synthetases and which comprises the 3′-hydroxyl group to which the amino acid is covalently attached to form an aminoacyl-tRNA;
(iv) the D stem-loop, consisting of a 4-6 bp stem and the D-loop that often contains dihydrouridine;
(v) the anticodon stem-loop, consisting of a 5 bp stem and the anticodon loop that contains the anticodon, which is complementary to an mRNA codon;
(vi) the variable loop, consisting of a loop which can vary in size from 3-21 nucleotides;
(vii) the T stem-loop, consisting of a 4-5 bp stem and the T loop, which contains the sequence TψC, where ψ is pseudouridine, an isomer of uridine.

The nucleotide residues of a tRNA may be numbered, according to a consensus numbering scheme that is well-known in the art, from the 5′-end to 3′-end, from N1 to N76. Typically, the residues of the 5′-acceptor stem are numbered N1-N7. The residues between the acceptor stem and the D stem-loop are numbered N8-N9. The residues of the 5′-D stem are numbered N10-N13. The residues of the D-loop are numbered N14-N21. The residues of the 3′-D stem are numbered N22-N25. The residues between the D stem-loop and anticodon stem-loop are numbered N26. The residues of the 5′-anticodon stem are numbered N27-N31. The residues of the anticodon loop are numbered N32-N38. The residues of the 3′-anticodon stem are numbered N39-N43. The residues of the variable loop are numbered N44-N48. The residues of the 5′-T stem are numbered N49-N53. The residues of the T loop are numbered N54-N60. The residues of the 3′-T stem are numbered N61-N65. The residues of the 3′-acceptor stem are numbered N66-N72. The residues of the 3′-NCCA tail are numbered N73-N76. Typically, tRNAs vary in length from between about 72-93 nucleotides and the structure of tRNAs also varies. Thus, depending on the particular tRNA, not all of the residues are always present and additional nucleotides may be present. The absence of a nucleotide is indicated by the absence of a number. An additional nucleotide is indicated by the number of the preceding nucleotide followed by a letter starting from “a”. The location of missing and additional nucleotides may be determined by comparison to a tRNA consensus sequence. This may be illustrated by the numbering of the E. coli tRNA^{Ser}GCU variable loop, which contains 21 nucleotides, numbered from 5′ to 3′ as N44 to N48. Specifically: N44, N45, N46, N47, N47a, N47b, N47c, N47d, N47e, N47f, N47g, N47h, N47i, N47j, N47k, N47l, N47m, N47n, N47o, N47p, N48.

The oligonucleotides of the present invention may hybridise to any region of the tRNA of interest, when said tRNA is in a folded state. The oligonucleotides of the present invention may hybridise to a region of the tRNA comprising the 3′ CCA tail, the D loop, the anticodon loop, the variable loop and/or the T loop. In some preferred instances the oligonucleotides hybridise to a region of the tRNA spanning from the anticodon stem-loop to the variable loop of the tRNA of interest. In some instances the oligonucleotides hybridise to the region of the tRNA spanning from the T-loop to the 3′-CCA end. In some instances the oligonucleotides hybridise to the region of the tRNA spanning from the anticodon stem-loop to the T stem-loop. In some instances the oligonucleotides hybridise to the region of the tRNA spanning from the D stem-loop to the anticodon stem-loop. In some instances the oligonucleotides hybridise to the region of the tRNA spanning from the 5′ end to the D loop. In some instances the oligonucleotides hybridise to the region of the tRNA spanning from the acceptor stem to the variable loop. In some instances the oligonucleotides hybridise to the region of the tRNA spanning from the D stem-loop to the variable loop. In some instances the oligonucleotides hybridise to the region of the tRNA comprising the acceptor stem and the anticodon loop. In some instances the oligonucleotides hybridise to the region of the tRNA comprising the D loop and the T loop. In some instances the oligonucleotides hybridise to the region of the tRNA comprising the anticodon loop and the T loop.

Where the oligonucleotides are described as hybridising to a region of the tRNA “spanning from X to Y” (e.g., spanning from nucleotides N1 to N76 of the tRNA of interest; or spanning from the anticodon stem-loop to the variable loop of the tRNA of interest), this may be understood to mean that the oligonucleotides may hybridise to any portion of the region between X and Y, including X and/or Y. For example, the oligonucleotides may hybridise only at X and/or Y, preferably at both X and Y. Alternatively, the oligonucleotides may hybridise across the whole length of the region between X and Y. In some instances, the oligonucleotides may initially hybridise at X and/or Y, but eventually hybridise across the whole length of the region between X and Y upon formation of a stable duplex between the oligonucleotide and the tRNA.

In preferred instances, the oligonucleotides hybridise to the region of the tRNA spanning from the D stem-loop to the anticodon stem-loop; or the region of the tRNA spanning from the D stem-loop to the variable loop; or the region of the tRNA spanning from the anticodon stem-loop to the variable loop; or the region of the tRNA spanning from the anticodon stem-loop to the T stem-loop; or the region of the tRNA spanning from the T stem-loop to the CCA-end.

In some instances, the oligonucleotides hybridise to a region of the tRNA of interest comprising nucleotides N8-N36, N31-N53, N34-N47h, N34-N47j, N34-N59, N53-N76 or N56-N76. In some preferred instances, the oligonucleotides hybridise to a region of the tRNA of interest comprising nucleotides N31-N53, N34-N47j or N56-N76. In some instances, the oligonucleotides hybridise to a region of the tRNA of interest comprising nucleotides equivalent to nucleotides N8-36, N34-47j, N34-47h, N34-47e, N38-47j, or N45-47j of E. coli tRNA^{Ser}_{GCU}. In some instances, the oligonucleotides hybridise to a region of the tRNA of interest comprising nucleotides equivalent to nucleotides N13-36, N31-53, N34-59, or N53-76 of E. coli tRNA^{Arg}_{CCU}. In some instances, the oligonucleotides hybridise to a region of the tRNA of interest comprising nucleotides equivalent to nucleotides N13-38, N32-47e, N21-40, N56-76, N1-21, or N47-59 of L. tarentolae tRNA^{Ser}_{GCU}. In some preferred instances, the oligonucleotides hybridise to a region of the tRNA of interest comprising nucleotides equivalent to nucleotides N34-47j of E. coli tRNA^{Ser}_{GCU}, N31-53 of E. coli tRNA^{Arg}_{CCU}, or N56-76 of L. tarentolae tRNA^{Ser}_{GCU}. Most preferably, the oligonucleotides hybridise to the region of the tRNA spanning from the anticodon stem-loop to the variable loop (N34-47j) of E. coli tRNA^{Ser}_{GCU}, to the region of the tRNA spanning from the anticodon stem-loop to the T stem-loop (N31-53) of E. coli tRNA^{Arg}_{CCU}, or to a region of the tRNA spanning from the T stem-loop to the 3′ CCA-end (N56-76) of L. tarentolae tRNA^{Ser}_{GCU}.

In some instances the oligonucleotides of the invention comprise a sequence that hybridises to, is complementary to, or is substantially complementary to, a sequence comprised within an endogenous or naturally occurring tRNA or a fragment thereof. The sequence comprised within an endogenous or naturally occurring tRNA may be at least 10, 15, 20, 25, 30, 35, 40, 45, 50, 55, 60 nucleotides in length, optionally the sequence may be between 10-20, 10-30, 10-40, 10-50, 10-60, 20-60, 20-50, 20-40, 30-40, 30-60, 20-45, 30-45, 30-55 or 35-55 nucleotides in length. Preferably the sequence comprised within an endogenous or naturally occurring tRNA may be between 10-60 nucleotides in length, most preferably between 15-30 nucleotides and 45-55 nucleotides in length. In some instances, the minimum length of complementarity between the tRNA of interest and the oligonucleotide of the invention is 10, 15, 20, 25 or 30 base-pairs in length, optionally the minimum length of complementarity is between 10-20, 10-30, 15-20, 15-30, 20-30, 10-40, 20-40, 30-40, 10-50, 20-50 or 30-50 base pairs in length, optionally between about 10-40 base pairs in length, preferably between about 10-30 base pairs in length.

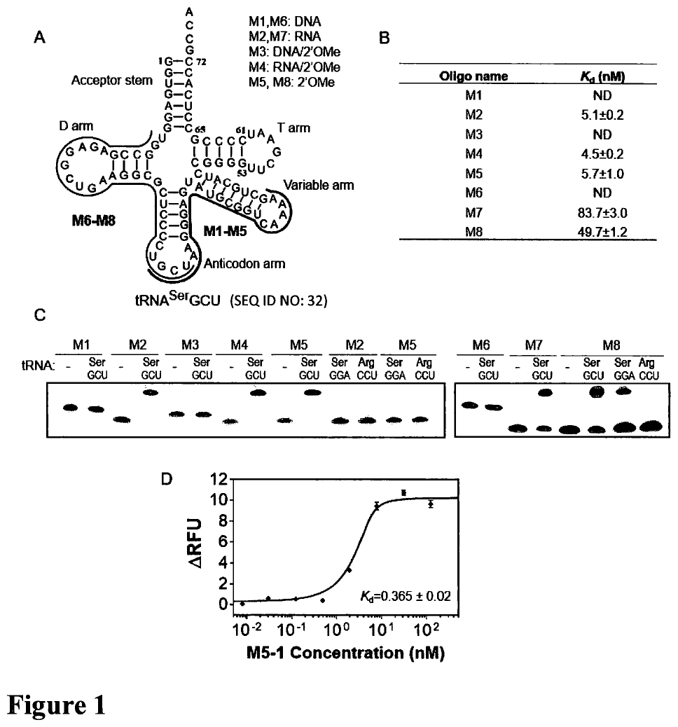

In some instances the oligonucleotide of the invention is exactly complementary to a region of at least 2, 3, 4, 5, 6 7, 8, 9 or 10 nucleotides of the variable loop. Hybridisation may comprise the formation of a stable duplex, which may be detected using denaturing PAGE analysis (e.g., see Example 1; FIG. 1C) or by the ability of the oligonucleotide to disrupt the function of the tRNA. In some instances the oligonucleotides of the invention form a stable duplex (i.e., base pair, either through Watson-Crick or wobble base pairing) with at least the variable loop and stem, preferably the oligonucleotides of the invention form a stable duplex with at least the variable loop, the variable stem and half of the anticodon stem. In preferred embodiments the oligonucleotides of the present invention form a stable duplex with residues N34-47j or N38-47j of the tRNA of interest.

In some instances the oligonucleotides of the present invention bind to the tRNA of interest with high affinity. The oligonucleotides may bind to the tRNA of interest with a Kd of between about 0.001-1500 nM, 0.01-1000 nM, 0.05-500 nM, 0.5-100 nM, 0.5-60 nM, 1-60 nM, 5-60 nM or 10-60 nM. In some instances the oligonucleotide may bind to the tRNA of interest with a Kd of between 0.001-1000 nM, 0.001-500 nM, 0.005-100 nM, 0.01-50 nM, 0.01-20 nM, 0.01-10 nM, 0.01-1 nM, 0.01-0.5 nM or 0.01-0.05 nM. In some instances the oligonucleotide may bind to the tRNA of interest with a Kd of less than 0.05 nM, 0.1 nM, 0.5 nM, 1 nM, 5 nM, 20 nM, 50 nM or less than 100 nM. In some instances the oligonucleotide may bind to the tRNA of interest with a Kd of between 0.1-100 nM. Preferably, the oligonucleotide may bind to the tRNA of interest with a Kd of between 0.1-1.5 nM (e.g., for 2′-O-methylated oligonucleotides of the invention) or between 10-60 nM (e.g., for non-methylated (i.e., unmodified) oligonucleotides of the invention). In some instances the oligonucleotide may bind to the tRNA of interest with a Kd of between 0.01-10 nM, preferably between 0.1-10 nM. In some instances the binding affinity or Kd is measured by microscale thermophoresis (MST), for example, as described in Example 1 and as shown in FIGS. 1B and D and 7.

In some instances the oligonucleotides of the present invention bind specifically to a particular tRNA of interest, i.e., they show reduced binding affinity for all other endogenous tRNAs. For example, the binding affinity of the oligonucleotide of the invention for a tRNA that is not the tRNA of interest may be at least 10, 20, 30 40, 50, 60, 70, 80, 90 or 100 times less than the binding affinity for the tRNA of interest, preferably at least 30 times less. The specificity of binding may be tested using any suitable method known in the art, for example such as fluorescence anisotropy, surface plasmon resonance, Microscale thermophoresis (MST) analysis or polyacrylamide gel electrophoresis (PAGE) gel shifting assays, as described in Example 1 and as shown in FIG. 1C.

Disrupting the Function of a tRNA

The oligonucleotides of the invention hybridise to a tRNA of interest when said tRNA is in a folded state, thereby disrupting function of the tRNA. Thus, the oligonucleotides of the invention are able to disrupt one or more functions of a tRNA of interest. In the process of protein translation each tRNA functions to decode a specific codon(s) in an mRNA sequence to a specific amino acid in the translated polypeptide. Each tRNA is linked to a specific amino acid in a reaction catalysed by a specific aminoacyl transferase. Each tRNA comprises an anticodon that hybridises to (recognises/base-pairs with) a specific codon(s) in an mRNA sequence either through Watson-Crick or wobble base pairing. Each tRNA binds to tRNA binding sites in the ribosome (the A, P, E, T and I sites) to deliver the amino acid to the growing polypeptide chain. Hybridisation of the oligonucleotide to the tRNA of interest may disrupt the tertiary structure or folded state of the tRNA and/or may disrupt the function of the tRNA in protein translation. The function of a tRNA may be defined as the ability to decode a specific mRNA codon to deliver an amino acid to the developing polypeptide chain during protein translation. Thus, the oligonucleotides of the invention hybridise to a tRNA of interest, when said tRNA is in a folded state, and may thereby disrupt the function of the tRNA in protein translation.

In some instances, the oligonucleotides of the invention change the structure of the tRNA, optionally disrupt the tertiary structure of the tRNA or disrupt the folded state of the tRNA. In some instances, the oligonucleotides of the invention linearize (i.e., uncoil, unfold, or unwrap) a portion of the tRNA of interest. In some instances, the oligonucleotides of the invention prevent binding of the tRNA of interest to its aminoacyl-tRNA transferase. In some instances, the oligonucleotides of the invention prevent binding of the tRNA of interest to elongation factor. In some instances, the oligonucleotides of the invention prevent binding of the tRNA of interest to the ribosome, optionally to the ribosome tRNA binding sites, optionally to any or all of the A, P, E, T and/or I sites of the ribosome. In some instances, the oligonucleotides of the invention prevent hybridisation of the anticodon of the tRNA of interest to its corresponding codon in the mRNA. Thus, the oligonucleotides of the invention inactivate the translational function of the tRNA of interest.

In some preferred instances, the oligonucleotides of the invention reduce translation of an mRNA comprising a codon that is recognised by (hybridises to/base-pairs with) the tRNA of interest and reduce the formation of the encoded translation product. As used herein, the term “reduces translation of an mRNA” is used to mean reduction of translation over the full length of the mRNA, i.e., reduced translation of the full-length protein product (i.e., the product of the translation reaction). The translation product whose formation is reduced may thus be the full-length protein encoded by the mRNA comprising at least one codon that is recognised by the tRNA of interest. The position of said codon in the mRNA dictates the position of the natural amino acid that is encoded by said codon in the protein translation product. Thus, the translation product whose formation is reduced may also be any translation product comprising the amino acid encoded by the targeted codon at the relevant position in the polypeptide chain. The skilled person is able to determine the position of the amino acid in the protein translation product that corresponds to the codon that is recognised by the tRNA of interest, from the mRNA sequence.

Translation of the mRNA may be measured in an in vitro translation reaction, for example using a cell lysate. In some instances, treatment of a cell lysate (e.g., an S30 lysate) with an oligonucleotide of the invention reduces translation of the full-length mRNA by at least 10%, 20%, 30%, 40%, 50%, 60%, 70%, 80%, 90% or at least 95% or substantially 100%, preferably between 60-100%, most preferably at least 90%, as compared to an untreated cell lysate. Alternatively, a cell lysate (e.g., an S30 lysate) that has been treated with an oligonucleotide of the invention may retain less than 90%, 80%, 70%, 60%, 50%, 40%, 30%, 25%, 20%, 15%, 10% or less than 5% or substantially 0%, preferably between 40-0%, most preferably less than 25% translation activity as compared to the translational activity of an untreated cell lysate. In some instances, treatment of a cell lysate with an oligonucleotide of the invention reduces the amount of the translation product (as defined herein) produced, for example after a pre-determined amount of time under conditions suitable for translation, by at least 10%, 20%, 30%, 40%, 50%, 60%, 70%, 80%, 90% or at least 95% or substantially 100%, preferably between 60-100%, most preferably at least 90%, as compared to an untreated cell lysate. Alternatively, in some instances a cell lysate (e.g., an S30 lysate) that has been treated with an oligonucleotide of the invention may produce less than 90%, 80%, 70%, 60%, 50%, 40%, 30%, 25%, 20%, 15%, 10% or less than 5% or substantially 0%, preferably between 40-0%, most preferably less than 25% the amount of the translation product (as defined herein) produced, for example after a pre-determined amount of time under conditions suitable for translation, as compared to the amount of translation product produced by an untreated cell lysate.

The % translational activity of an in vitro translation system may be measured as described in Examples 2, 4 and 5. For example, translational activity of the lysate towards a GFP template comprising 1 or 2 codons that are recognised by the tRNA of interest may be monitored by the fluorescence. Therefore, an S30 lysate incubated with 2 μM, 4 μM, 8 μM or 16 μM of an oligonucleotide of the present invention at 37° C. for 5 minutes, assembled into an in vitro translation reaction wherein the final concentration of the oligonucleotide of the invention is approximately 0.8 μM, 1.6 μM, 3.2 μM, 6.4 μM, respectively, may retain less than 25%, preferably less than 10%, translation activity of a GFP template containing 2 consecutive codons that are recognised by the tRNA of interest as compared to an untreated S30 lysate, as monitored by GFP fluorescence. GFP fluorescence can be measured by techniques well known in the art, for example by fluorescence spectroscopy, for example, measuring the fluorescence emission levels at 528 nm from the translated GFP protein following excitation at 485 nm.

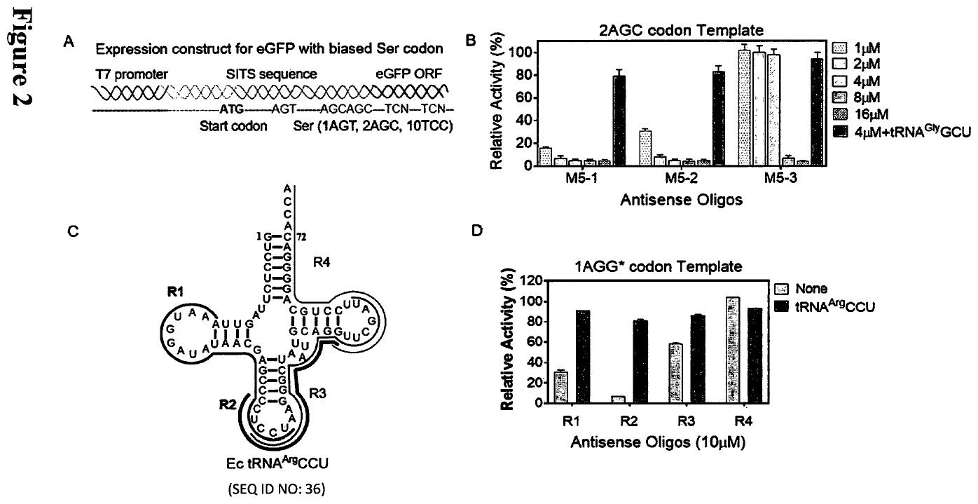

In some instances, the reduction in translational activity may be rescued and production of the full-length protein restored by adding a tRNA that does not hybridise to the oligonucleotide of the present invention, that is linked to a natural or unnatural amino acid (as defined later herein) and that recognises the same codon as the tRNA of interest. An increase in the production of the GFP protein (as measured by an increase in the fluorescence) indicates that the reduction in the translational activity is due to specific inactivation of the tRNA of interest by the oligonucleotide of the invention. For example, as described in Example 2 and as shown in FIG. 2B, the reduction in translational activity and decreased production of GFP protein from a template comprising 2 AGC codons that resulted from specific inactivation of tRNA^{Ser}_{GCU} using an oligonucleotide of the invention, could be rescued by addition of tRNA^{Gly}_{GCU}, which restored translation of GFP protein as indicated by increased fluorescence. In some instances, the translational activity may be recovered to at least 70%, 80%, 85%, 90%, 91%, 92%, 93%, 94%, 95%, 96%, 97%, 98%, 99% or at least 100%, preferably between 80-100%, most preferably at least 80% translational activity as compared to the translational activity of a cell lysate that has not been treated with the oligonucleotides of the invention.

The present invention also provides the use of an oligonucleotide of the invention in a method for disrupting the function of a tRNA of interest. The present invention also provides the use of an oligonucleotide of the invention in a method for producing a polypeptide comprising at least one unnatural amino acid. The present invention also provides the use of an oligonucleotide of the invention in a method for reassigning a codon to a unnatural amino acid.

Methods for Disrupting Function of a tRNA of Interest

As described above, the oligonucleotides of the invention hybridise to a tRNA of interest when said tRNA is in a folded state, and thereby disrupt the function of the tRNA. Therefore, the present invention also provides a method for disrupting function of a tRNA of interest, the method comprising contacting the tRNA in a folded state with an oligonucleotide of the present invention that hybridises to the tRNA in said state, thereby disrupting function of the tRNA.

The tRNA of interest may be in solution or suspension, preferably solution. In some instances, the tRNA of interest may be in a buffered solution, preferably the tRNA of interest is in a cell lysate or an in vitro translation system. In some instances, the oligonucleotide of the invention is also in solution or suspension, preferably in solution. The oligonucleotide of the present invention may be added to the solution comprising the tRNA of interest. In preferred instances the tRNA of interest is incubated with the oligonucleotide of the invention, optionally for at least 1, 2, 3, 4, 5, 10, 20 or at least 30 minutes. Preferably, the tRNA of interest is incubated with the oligonucleotide of the invention for between 5-10 minutes. In some instances, the oligonucleotide of the invention may be immobilised on a solid support. In some instances the oligonucleotide of the invention is not attached to a solid support. The solid support may comprise a nanoparticle, a bead, a membrane, a mesh or a matrix. Preferably the oligonucleotide is in solution; preferably in a buffered solution. In some instances, the contacting of the tRNA of interest and the oligonucleotide of the invention occurs in solution or suspension, preferably in solution; more preferably in a buffered solution, a cell lysate or an in vitro translation system. Most preferably the contacting of the tRNA of interest and the oligonucleotide of the invention occurs in a buffered solution or a cell lysate.

In some instances, the contacting is performed under non-denaturing conditions, optionally under physiological conditions. The term “non-denaturing conditions” is used herein to mean that the contacting is performed in the absence of reducing agents and not at extremes of temperature and/or pH. Examples of extreme temperatures includes any temperature below 0° C. or above 100° C. Examples of extreme pH includes any pH lower than pH 2 and higher than pH 10. In some instances the contacting is carried out in non-denaturing conditions wherein the temperature is about 5° C., 10° C., 15° C., 20° C., 25° C., 30° C., 35° C., 37° C., 40° C., 45° C., 50° C., 55° C. or 60° C., optionally between about 10° C.-50° C., 15° C.-45° C., 20° C.-40° C., 25° C.-37° C. or 25° C.-40° C., preferably between about 20° C. to about 40° C. In some instances the contacting is carried out in non-denaturing conditions wherein the pH is about 3, 4, 5, 6, 7, 8, 9 or 10, optionally between about 5-9, 6-8, 6.5-7.5, 7-8 or 6.5-8.5, preferably between about 6 to about 8. In preferred instances the contacting is carried out in non-denaturing conditions that are physiological conditions and comprise a temperature of about 20° C. to about 40° C. and/or a pH of about 6 to about 8. In preferred instances, the tRNA of interest is not denatured during the contacting, i.e., the tRNA of interest is in a folded state as described above.

In some instances, the contacting is performed in a liquid, preferably water or a buffered solution. In some preferred instances the liquid comprises only water. In some other instances the liquid comprises a buffer, for example, a phosphate buffer, a HEPES buffer, a Tris buffer. In some instances the liquid comprises a salt, for example, an ionic halide, preferably NaCl or MgCl₂. In some instances, the contacting (i.e., of the tRNA of interest and the oligonucleotide of the invention) is performed in the presence of at least one other tRNA, optionally wherein the other tRNA is an endogenous or naturally occurring tRNA. In some instances, the contacting is performed in the presence of a plurality of tRNAs. In some instances, the contacting is performed in the presence of the full complement of natural tRNAs, which includes the tRNA of interest. In some instances, the contacting is performed in the presence of the full complement of natural tRNAs. In some instances, the contacting is performed in the presence of a plurality of tRNAs suitable for incorporation of all of the twenty natural amino acids. Thus, the contacting is typically performed without purification of the tRNA of interest away from other tRNAs. In some preferred instances, the contacting is performed in a cell lysate.

The term “full complement of natural tRNAs” is used herein to mean all of the tRNAs naturally occurring in a particular organism of interest. For example, in some instances a total native tRNA mixture isolated or purified from E. coli cells or Leishmania tarentolae cells (e.g., by phenol extraction or using an ethanolamine matrix, as described in Example 5) comprises the full complement of E. coli tRNAs or Leishmania tarentolae tRNAs, i.e., all of the naturally occurring E. coli tRNAs or Leishmania tarentolae tRNAs. In some instances a lysate obtained from E. coli cells would comprise the full complement of E. coli tRNAs, i.e., all of the naturally occurring E. coli tRNAs. Thus, the full complement of tRNAs can be defined as all of the tRNAs present in (i) a cell lysate produced by lysing the cells of a selected organism, or (ii) a purified total tRNA mixture, optionally in water. Thus, a solution comprising a full complement of tRNAs may be prepared by purifying all of the tRNAs present in a cell lysate. For example, lysis of E. coli cells results in a cell lysate comprising the full complement of endogenous E. coli tRNAs, which may then be purified from the cell lysate. A full complement of tRNAs may be isolated from a cell lysate by methods well-known to the person skilled in the art, for example, phenol extraction or ethanolamine sepharose chromatography.

In some instances the contacting is performed in the presence of all of the necessary components for protein translation. For example the contacting may be performed in the presence of purified ribosomes and/or the protein factors for translation and/or the protein factors for transcription. These protein factors may comprise transcriptional and/or translational initiation, elongation and termination factors. In some preferred instances, the contacting is performed in a cell lysate. In some instances the contacting is performed under conditions suitable for translation. For example, the contacting may be performed in the presence of the necessary components for protein translation, an energy generating system (such as creatine kinase), a substrate (e.g., mRNA), amino acids and buffers. In some instances, the contacting is performed in a cell-free translation system or a liquid comprising a cell-free translation system. In some instances, the contacting is performed in a reconstituted in vitro cell-free translation system or a liquid comprising a reconstituted in vitro cell-free translation system.

A “cell lysate” refers to the composition produced by lysing cells of a selected organism of interest. A cell lysate comprises the translational machinery that is required for translation as described herein, including ribosomes and protein factors. An “in vitro translation system” or “cell-free translation system” refers to a composition having the capability of performing a translation reaction. An in vitro translation system provides conditions suitable for translation. An in vitro translation system may be assembled, for example, by supplementing a cell lysate with other components necessary for translation, including, for example, an energy generating system (for example Creatine kinase and phosphocreatine), mRNA, amino acids, buffers and other components typically included in a standard cell free translation system, which would be well-known to the person skilled in the art. In some instances, transcription and translation may be coupled, in which case the in vitro translation system may further comprise an RNA polymerase (e.g., T7 polymerase), rNTPs, a DNA template for producing mRNA and/or DNA polynucleotides for producing RNA oligonucleotides of the invention.

In some instances, the contacting is performed in a liquid. In some instances, the contacting is performed in a cell lysate. In some instances, the contacting is performed in an in vitro translation system, optionally comprising a cell lysate. In some instances the contacting is performed in total tRNA mixture. Total tRNA mixture refers to a solution or suspension of tRNAs, and is typically purified from a cell lysate. The cell lysate can be prepared from the cells of any selected organism. Preferably the cell lysate is prepared from human cells, mammalian cells, bacterial cells, protozoan cells, such as Leishmania, or yeast cells. In preferred instances the cell lysate is prepared from the bacteria Escherichia coli or the trypanosome Leishmania tarentolae. As described above, in some instances the cell lysate or total tRNA mixture comprises a full complement of tRNAs. In some instances the cell lysate or total tRNA mixture comprises tRNAs suitable for incorporation of all of the twenty natural amino acids. The cell lysate or total tRNA mixture comprises the tRNA of interest, optionally wherein the tRNA of interest is one of the endogenous tRNAs, as described above.

### Methods for Synthesising Proteins Comprising Unnatural Amino Acids

As described above, the oligonucleotides of the invention hybridise to a tRNA of interest when said tRNA is in a folded state, and thereby disrupt the function of the tRNA. This frees the codon recognised by the tRNA of interest for re-allocation to code for an unnatural amino acid. Thus, the present invention further provides an in vitro method for producing a polypeptide comprising at least one unnatural amino acid, the method comprising incubating:

(a) an mRNA comprising a codon that is recognised by a tRNA of interest;
(b) an oligonucleotide of the invention that hybridises to the tRNA of interest when said tRNA is in a folded state, thereby disrupting the function of the tRNA; and
(c) a tRNA that (i) recognises the same codon as the tRNA of interest and (ii) is linked to an unnatural amino acid;
under conditions suitable for translation of said mRNA.

The present invention also provides an in vitro method for producing a polypeptide comprising at least one unnatural amino acid, the method comprising in a first step incubating:

(a) an oligonucleotide of the invention that hybridises to the tRNA of interest when said tRNA is in a folded state, thereby disrupting the function of the tRNA; and
(b) one or more tRNAs comprising the tRNA of interest, optionally a full complement of naturally occurring tRNAs which includes the tRNA of interest.

In some instances the one or more tRNAs or the full-complement of naturally occurring tRNAs is comprised in a cell lysate.

The method further comprises in a second step incubating:

(c) the mixture resulting from the first step of: (i) the oligonucleotide of the invention and (ii) the one or more tRNAs, or the full-complement of naturally occurring tRNAs, (optionally comprised within a cell lysate);
(d) an mRNA comprising a codon that is recognised by a tRNA of interest; and either:
(e) a tRNA that (i) recognises the same codon as the tRNA of interest and (ii) is linked to an unnatural amino acid; or
(e) an orthogonal tRNA that recognises the same codon as the tRNA of interest; an unnatural amino acid; and an orthogonal aminoacyl-tRNA synthetase suitable for charging the orthogonal tRNA with the unnatural amino acid;
under conditions suitable for translation of said mRNA.

In the context of this invention, the term “polypeptide” is used interchangeable with the term “protein” and refers to a polymer of amino acid residues linked by peptide or amide bonds. The polypeptides may comprise any or all of the twenty canonical amino acids (i.e., “naturally occurring” or “natural” amino acids), which include the L-enantiomers of Glycine, Alanine, Valine, Leucine, Isoleucine, Proline, Phenylalanine, Tyrosine, Tryptophan, Serine, Threonine, Cysteine, Methionine, Asparagine, Glutamine, Lysine, Arginine, Histidine, Aspartate (Aspartic acid) and Glutamate (Glutamic acid). The polypeptides may also comprise the naturally occurring but non-canonical (i.e., non-standard) amino acids pyrrolysine, selenocysteine or N-formylmethionine. The methods of the present invention may be used to produce any polypeptide of any length and any sequence.

The term “unnatural amino acid” (also referred to as “non-natural”, “non-canonical”, “non-standard”, “non-coding” or “non-proteinogenic” amino acids) as used herein refers to any molecule capable of incorporation into a protein translatable from an RNA template via ribosome-mediated chain elongation, with the proviso that it is not a “natural amino acid” as defined above. Unnatural amino acids may include natural or synthetic chemical derivatives of natural amino acids and/or chemically-reactive moieties such as moieties capable of forming intramolecular covalent bonds. The unnatural amino acid may be any organic compound comprising an amine (—NH₂) and a carboxyl (—COOH) functional group and that is capable of peptide bond formation. Non-limiting examples of unnatural moieties include any of the D-amino acids, such as D-serine, D-tyrosine, D-alanine, D-tryptophan; any N-methylated amino acid, such as N-methyl alanine, N-methyl β-alanine, N-methyl-leucine, N-methyl-valine; any fluorophore, such as BODIPY FL, which also comprises a functional group such that it is capable of peptide bond formation; selenocysteine, pyrrolysine, N-formylmethionine, a-Amino-n-butyric acid, norvaline, norleucine, alloisoleucine, t-leucine, a-Amino-n-heptanoic acid, pipecolic acid, α,β-diaminopropionic acid, α,γ-diaminobutyric acid, ornithine, allothreonine, homocysteine, homoserine, β-alanine, β-amino-n-butyric acid, β-aminoisobutyric acid, γ-aminobutyric acid, a-aminoisobutyric acid, isovaline, sarcosine, N-ethyl glycine, N-propyl glycine, N-isopropyl glycine, N-ethyl alanine, N-ethyl β-alanine, N-chloroacetyl-methionine, N-chloroacetyl-tryptophan, p-propargyloxyphenylalanine, p-azidophenylalanine (AzF), p-propargyloxyphenylalanine, or n-propargyllysine (PrK). The skilled person would be capable of identifying a suitable unnatural amino acid that may be incorporated into a protein using the methods of the present invention. A polypeptide comprising any unnatural amino acid may be produced using the methods of the present invention. In preferred instances, a polypeptide comprising the unnatural amino acids azidophenylalanine (AzF) and/or propargylysine (PrK) is produced using the methods of the present invention.

The polypeptides produced by the methods of the present invention comprise at least one unnatural amino acid. In some instances, the polypeptides produced by the methods of the present invention comprise 1, 2, 3, 4, 5, 6, 7, 8, 9, 10, 11, 12, 13, 14, 15, 16, 17, 18, 19 or 20 unnatural amino acids. In some instances, the polypeptides produced by the methods of the present invention comprise between 1-5, 1-10, 1-20, 5-10, 5-20 or between 10-20; preferably between 1-10 unnatural amino acids. In some instances, the polypeptides produced by the methods of the present invention comprise up to 1%, 2%, 3%, 4%, 5%, 10%, 15%, 20%, 25%, 30%, 35%, 40%, 50%, 60%, 70%, 80%, 90% or substantially 100% unnatural amino acids. In some instances, the polypeptides produced by the methods of the present invention comprise between 1%-5%, 1%-10%, 5%-10%, 1%-20%, 5%-20%, 10%-20%, 1%-50%, 10%-50%, 20%-50%, 1%-100%, 10%-100%, 20%-100%, 50%-100%; preferably between 1%-20% unnatural amino acids. In some instances, the polypeptides produced by the methods of the present invention comprise one type of unnatural amino acid. In some instances, the polypeptides produced by the methods of the present invention comprise at least one type of unnatural amino acid. In some instances, the polypeptides produced by the methods of the present invention comprise at least two different types of unnatural amino acid. In some instances, the polypeptides produced by the methods of the present invention comprise at least three, at least four or at least five different types of unnatural amino acids. Furthermore, the in vitro methods for producing a polypeptide comprising at least one unnatural amino acid of the present invention may also be used for producing a polypeptide comprising a canonical amino acid. This may be done, for example, by providing a tRNA that (i) recognises the same codon as the tRNA of interest and (ii) is linked to a canonical amino acid.

The “mRNA”, as used in the methods described herein, refers to any mRNA that is capable of being translated to produce a polypeptide under conditions suitable for translation, e.g., an in vitro translation system. Typically, the mRNA comprises or encodes an open reading frame (“ORF”). The mRNA typically comprises or consists of RNA nucleotides. The mRNA may be any length and may comprise any sequence. The mRNA comprises at least one codon that is recognised by a tRNA of interest, wherein the tRNA of interest is defined as described herein above. The mRNA may comprise a promoter (e.g., the T7 promoter), a species-independent translational leader sequence (SITS) or a ribosomal binding site (e.g., the Shine-Dalgarno sequence), an initiation codon (e.g., AUG) and/or a stop codon (e.g., UAA, UGA or UAG).

An mRNA “codon” typically comprises three (i.e., a triplet of) RNA nucleotides. Each codon encodes a natural amino acid or serves as a translation terminator (the terminator codons are UAA, UGA, UAG). An amino acid may be encoded by multiple codons. Each codon is recognised by an anticodon in a tRNA, i.e., the anticodon of the tRNA hybridises, either through Watson-Crick or wobble base pairing, to a specific codon in the mRNA. Typically, the anticodon is a 3′-5′ tri-nucleotide sequence that forms Watson-Crick base pairs at least at the first and second positions with the corresponding 5′-3′ mRNA codon sequence. Each tRNA recognises a specific codon and decodes that codon to a particular amino acid. Since the genetic code is degenerate there may be more than one tRNA decoding a codon box for a given amino acid. Such tRNAs decoding the same amino acid and comprising different anticodons are defined as “isoacceptors”. In the case of all codons, except those encoding Met and Trp, the specificity of base-pairing between the anticodon and the mRNA codon is defined by the first two nucleotides (i.e., read 3′-5′ in the anticodon and 5′-3′ in the mRNA codon), such that the same anticodon may recognise two or more degenerate mRNA codons. Each of the single tRNA-isoacceptors for Asp, Asn, Cys, Glu, His, Lys, Phe and Tyr recognise two different mRNA codons (i.e., “two-fold degenerate”). Two tRNA-isoacceptors for Gln recognise two different mRNA codons (i.e., “two-fold degenerate”) (for example, one of the isoacceptors recognises both of the mRNA codons encoding Gln and the other isoacceptor only recognises one of the mRNA codons encoding Gln). Ile has two isoacceptors, one that recognises two different mRNA codons and one that recognises a single mRNA codon (i.e., “three-fold degenerate”). There are two isoacceptors for Ala and Val and three isoacceptors for Gly, Pro and Thr that recognise respective codons in an RNA template (i.e., “four-fold degenerate”). For Arg, Leu and Ser there are four or five isoacceptors that recognise respective codons (i.e., “six-fold degenerate”).

The mRNA comprises at least one codon that is recognised by (i.e., that hybridises to) the anticodon of the tRNA of interest. The mRNA may comprise 1, 2, 3, 4, 5, 6, 7, 8, 9, 10, 11, 12, 13, 14, 15, 16, 17, 18, 19 or 20 codons that are recognised by the tRNA of interest. In some instances the mRNA comprises one codon that is recognised by the tRNA of interest. In some instances the mRNA comprises at least one codons that are recognised by the tRNA of interest. In some instances the mRNA comprises two codons that are recognised by the tRNA of interest. In some instances the mRNA comprises at least two codons that are recognised by the tRNA of interest. In some instances the codon is CGU, CGC, CGA, CGG, AGA, AGG, UUA, UUG, CUU, CUC, CUA, CUG, UCU, UCC, UCA, UCG, AGU or AGC. In some preferred instances, the codon is AGC, AGU or AGG.

Similarly, the tRNA of interest comprises an anticodon that recognises (i.e., hybridises to) at least one codon in the mRNA. An oligonucleotide of the present invention, as described herein above, hybridises to the tRNA of interest, as described herein above, when said tRNA is in a folded state, thereby disrupting the function of the tRNA. Thus, the tRNA of interest can no longer recognise (i.e., hybridise to) the codon in the mRNA. This codon may then be reassigned to encode an unnatural amino acid using the methods of the present invention. Thus, the mRNA comprises at least one codon that has been reassigned to encode an unnatural amino acid.

In some preferred instances, the mRNA used in any of the methods described herein is produced by transcription of a DNA template. Thus, in some preferred instances, any of the methods described herein further comprise a step prior to step (a) wherein a DNA template is transcribed to produce the mRNA. Similarly, where the oligonucleotides of the present invention used in any of the methods described herein are RNA oligonucleotides, the RNA oligonucleotides of the present invention may be produced by transcription of a DNA polynucleotide, as described earlier herein. Thus, in some instances, any of the methods described herein further comprise a step prior to step (a) wherein (i) a DNA template is transcribed to produce the mRNA and/or (ii) a DNA polynucleotide of the invention is transcribed to produce the RNA oligonucleotides of the invention. In these instances the transcription reaction may be performed separately. However, in preferred instances, the transcription and translation reactions will be coupled. Thus, in these instances, the methods described herein may be performed under conditions suitable for transcription and translation. Such conditions are described in more detail herein.

As described above, the oligonucleotides of the present invention hybridise to and disrupt the function of a tRNA of interest. The codon recognised by this tRNA of interest is then freed for decoding by the tRNA that (i) recognises the same codon as the tRNA of interest and (ii) is linked to an unnatural amino acid, or the orthogonal tRNA that recognises the same codon as the tRNA of interest and that is linked to an unnatural amino acid, following charging with an unnatural amino acid by an orthogonal aminoacyl tRNA synthetase. The tRNA or orthogonal tRNA recognises the same codon as the tRNA of interest. The tRNA or orthogonal tRNA therefore recognises (i.e., hybridise to) the codon that was recognised by the now inactivated tRNA of interest. The tRNA or orthogonal tRNA decodes said codon and delivers the unnatural amino acid to the developing polypeptide chain for incorporation into the polypeptide. The tRNA or orthogonal tRNA does not hybridise to the oligonucleotide of the invention and is not disrupted in function by the oligonucleotide of the invention. In some preferred instances, the tRNA that (i) recognises the same codon as the tRNA of interest and (ii) is linked to an unnatural amino acid, referred to herein as “the tRNA”, may be an orthogonal tRNA.

The term “orthogonal tRNA” as used herein may refer to a tRNA that has the same structural features as endogenous or naturally occurring tRNAs, including for example, the same folded structure (e.g., the compact L-shape) and the same functional properties (e.g., binding to ribosomes and elongation factor, delivery of amino acids to the developing polypeptide chain), but that is orthogonal to the biosynthetic machinery. The orthogonal tRNA may be not naturally occurring (i.e. not found in nature), for example, the orthogonal tRNA may be synthetic. The term “orthogonal” as used herein means not encoded in the genome of the organism of interest, or not endogenous to the organism of interest. The term “organism of interest” as used herein means the organism or cell type (i) that was used to make the cell extract or lysate used in the methods described herein; or (ii) from which the total native tRNA mixture used in the methods described herein is purified; or (iii) from which the tRNA of interest used in the methods described herein originates or is purified; or (iv) from which the one or more tRNAs used in the methods described herein originate or are purified. The following components of the translation reaction may be endogenous to (encoded in the genome of) the “organism of interest”: the tRNA of interest, the one or more tRNAs used in the translation reaction, the full complement of tRNAs, the cell lysate and/or the necessary components for translation. Orthogonal is used herein to mean: does not interact with the endogenous amino acid charging system of the organism of interest. For example, an orthogonal tRNA may not be charged with an amino acid by any of the endogenous (i.e., naturally genetically encoded) aminoacyl tRNA synthetases.

The orthogonal tRNA is thus either not encoded by the genome of any organism, and not part of the naturally occurring complement of tRNAs (e.g., a synthetic tRNA), or is orthogonal, i.e., not encoded by the genome of the organism of interest. In some instances, the orthogonal tRNA is not able to be charged with an amino acid by an endogenous or a naturally occurring aminoacyl tRNA synthetase. In some instances, the orthogonal tRNA is only able to be charged by an orthogonal aminoacyl tRNA synthetase. The orthogonal tRNA recognises the same codon as the tRNA of interest, i.e., hybridises to the same codon as the tRNA of interest either through Watson-Crick or wobble base pairing, as described above; and/or has an anticodon that is complementary or substantially complementary to the same codon as the tRNA of interest. In some preferred instances the orthogonal tRNA is orthogonal to the endogenous amino acid charging system, i.e., the orthogonal tRNA is not recognised by any endogenous aminoacyl tRNA synthetase (aaRS). An aminoacyl tRNA synthetase (aaRS) is an enzyme that attaches (charges, loads or aminoacylates) the tRNA with its appropriate amino acid. The orthogonal (or synthetic) tRNA may only be charged with an amino acid by an orthogonal (or synthetic) aminoacyl tRNA synthetase. The orthogonal aminoacyl tRNA synthetase is not encoded in the genome of the organism of interest and is not part of the endogenous complement of aminoacyl tRNA synthetases. In some instances, the orthogonal aminoacyl tRNA synthetase is not able to charge an endogenous or naturally occurring tRNA with any of the 20 canonical amino acids. In some instances, the orthogonal aminoacyl tRNA synthetase is only able to charge an orthogonal tRNA with an unnatural amino acid. An orthogonal tRNA:aminoacyl tRNA synthetase pair describes an orthogonal aminoacyl tRNA synthetase that specifically charges the orthogonal tRNA in the pair with an unnatural amino acid and will not charge any other natural tRNA with a canonical and/or unnatural amino acid.

In some instances, the method comprises incubating:

(a) an mRNA comprising a codon that is recognised by a tRNA of interest;
(b) an oligonucleotide of the invention that hybridises to the tRNA of interest when said tRNA is in a folded state, thereby disrupting the function of the tRNA; and
(c) a tRNA that (i) recognises the same codon as the tRNA of interest and (ii) is linked to an unnatural amino acid;
under conditions suitable for translation of said mRNA.

In some instances the tRNA is linked to an unnatural amino acid. In some instances the tRNA is charged with an unnatural amino acid, for example by amino acid acylation. The tRNA may be charged with an amino acid, for example, by in vitro enzymatic or chemical synthesis or by using orthogonal aminoacyl-tRNA synthetases during translation. In some instances the tRNA (i) recognises the same codon as the tRNA of interest (i.e., hybridises to the same codon as the tRNA of interest either through Watson-Crick or wobble base pairing or has an anticodon that is complementary or substantially complementary to the same codon as the tRNA of interest) and (ii) is linked to an unnatural amino acid. In some preferred instances the tRNA may be an orthogonal tRNA. In some instances, the tRNA may have a different nucleotide at the N34 position as compared to the tRNA of interest, which may enable it to recognise the target codon via Watson-Crick base pairing at all three codon-anticodon positions. Unnatural amino acids, as defined above herein, may be linked, coupled, charged or loaded onto the tRNA by any method known in the art, for example chemical aminoacylation or enzymatic aminoacylation. Non-limiting examples of enzymatic aminoacylation include the use of natural or modified aminoacyl tRNA synthases such as PylRS or variants thereof used in pyrrolysine tRNA synthase-mediated aminoacylation; Methanococcus jannaschii tyrosyl-transfer RNA synthetase (Mj TyrRS) or variants thereof; Flexizyme-mediated aminoacylation; and/or aminoacylation by a cysteinyl tRNA synthase followed by further chemical derivation of the thiol group.

In some instances, the tRNA may be linked to an unnatural amino acid in a reaction catalysed by an aminoacyl tRNA synthetase. In some instances, the aminoacyl tRNA synthetase is orthogonal to the endogenous amino acid charging system, i.e., the aminoacyl tRNA synthetase does not interact with any components of the amino acid charging system found in the organism; i.e., the aminoacyl tRNA synthetase does not recognise any endogenous tRNAs or naturally occurring amino acids. In some instances the tRNA and the aminoacyl tRNA synthetase form an orthogonal pair, i.e., both are orthogonal to the endogenous amino acid charging system, but the aminoacyl tRNA synthetase will specifically aminoacylate the tRNA with an unnatural amino acid. In some instances, the unnatural amino acid is linked to the tRNA at the 2′-OH or the 3′-OH group of the tRNA nucleotide at the 3′-end of the tRNA.

In some instances, the tRNA, the aminoacyl tRNA synthetase and the unnatural amino acid are incubated together, to produce the tRNA linked to the unnatural amino acid, prior to incubation with the mRNA and the oligonucleotide of the invention. The resulting tRNA linked to an unnatural amino acid is then incubated with the mRNA and the oligonucleotide of the invention, under conditions suitable for translation of said mRNA. Alternatively, in some instances, the tRNA, which is preferably an orthogonal tRNA, the aminoacyl tRNA synthetase and the unnatural amino acid are incubated together with the mRNA and the oligonucleotide of the invention, under conditions suitable for translation of said mRNA. Thus, the tRNA may be linked to an unnatural amino acid pre-translationally or co-translationally.

Thus, in some alternative instances, the method comprises incubating:

(a) an mRNA comprising a codon that is recognised by a tRNA of interest;
(b) an oligonucleotide of the invention that hybridises to the tRNA of interest when said tRNA is in a folded state, thereby disrupting the function of the tRNA;
(c) an orthogonal tRNA that recognises the same codon as the tRNA of interest;
(d) an unnatural amino acid; and
(e) an orthogonal aminoacyl-tRNA synthetase suitable for charging the orthogonal tRNA with the unnatural amino acid;
under conditions suitable for translation of said mRNA.

The skilled person would be capable of selecting suitable tRNAs that recognise the same codon as the tRNA of interest and that are preferably orthogonal to the endogenous amino acid charging system. The skilled person would also be capable of selecting an orthogonal aminoacyl-tRNA synthetase suitable for charging the tRNA with the unnatural amino acid. In particular, the skilled person would be capable of identifying orthogonal tRNA: aminoacyl tRNA synthetase pairs for use in the methods of the invention. The nature of the orthogonal aminoacyl-tRNA synthetase will depend on the structure of the unnatural amino acid. Typically, such orthogonal tRNA:aminoacyl tRNA synthetase pairs are developed by rounds of directed evolution to enable loading of a particular unnatural amino acid onto a tRNA. Non-limiting examples of such orthogonal tRNA: aminoacyl tRNA synthetase pairs include the MjTyr system and the Pyl system.

In some instances, the tRNA is tRNA^{MjY}_{GCU} or tRNA^{MjY}_{ACU}, the unnatural amino acid is azidophenylalanine (AzF) and the aminoacyl tRNA synthetase is AzFRS. In some instances, the tRNA is tRNA^{Pyl}_{ACU} or tRNA^{Pyl}_{GCU}, the unnatural amino acid is propargyllysine (PrK) and the aminoacyl tRNA synthetase is an engineered PylRS (PylRSAF).

The tRNA linked to an unnatural amino acid as described herein can also be described by reference to its function. As described in the Examples, following the disruption of the tRNA of interest by an oligonucleotide of the invention (as measured by a reduction in the translation activity of an eGFP mRNA template comprising at least one codon recognised by the tRNA of interest, as described above), a suitable (i) tRNA that recognises the same codon as the tRNA of interest and is linked to an unnatural amino acid or (ii) orthogonal tRNA that recognises the same codon as the tRNA of interest; unnatural amino acid; and orthogonal aminoacyl-tRNA synthetase suitable for charging the orthogonal tRNA with the unnatural amino acid; may be identified as those that restore translational activity of the eGFP to at least 10%, 20%, 30%, 40%, 50%, 60%, 70%, 80%, 90% or at least about 100% translational activity, preferably between 50-100% translational activity, as compared to an in vitro translation system comprising a cell lysate that has not been treated with an oligonucleotide of the invention (an untreated cell lysate). Suitable components may also be identified by their ability to increase the production of eGFP protein, as measured by an increase in the fluorescence. Furthermore, suitable components may also be identified by at least 10%, 20%, 30%, 40%, 50%, 60%, 70%, 80%, 90% or substantially 100%, preferably between 50%-100%, most preferably at least 60% of the protein produced in the method of the invention comprising the unnatural amino acid. The incorporation of an unnatural amino acid into a polypeptide may be measured, for example, using mass spectrometry, preferably LC-MS/MS.

The methods of the invention comprise incubating the components as described above under conditions suitable for translation of the mRNA. The term “conditions suitable for translation” as used herein, may refer to conditions suitable for translation of an mRNA to produce a polypeptide. The skilled person would be readily capable of assembling conditions suitable for translation, for example as described herein in the Materials and Methods of E. coli in vitro protein translation assay. For example, conditions suitable for translation may comprise at least one other tRNA, preferably the full complement of natural tRNAs, which includes the tRNA of interest; ribosomes and the necessary protein factors for translation (e.g., initiation factors (e.g., IF1, IF2, and IF3); elongation factors (e.g., EF-Tu, EF-G); release factors (e.g., RF1, RF2, RF-3)); energy sources (e.g., ATP, GTP); an energy regenerating system (for example, creatine phosphate and creatine kinase); amino acids; buffer; salts (e.g., Mg²⁺, K⁺, etc.) and other necessary components. In preferred instances, conditions suitable for translation comprises a cell lysate (e.g., from an organism of interest) supplemented with a feeding solution comprising energy sources (e.g., ATP, GTP); an energy regenerating system (for example, creatine phosphate and creatine kinase); amino acids; buffer; salts (e.g., Mg²⁺, K⁺, etc.) and other necessary components. In preferred instances, conditions suitable for translation comprise an in vitro (or cell-free) translation system.

In some instances, as described above, any of the method described herein may be performed under conditions suitable for transcription and translation (e.g., in instances where a DNA template is provided for producing the mRNA and/or where a DNA polynucleotide, as described herein, is provided for producing the RNA oligonucleotide of the invention). In such instances, transcription and translation are coupled. The term “conditions suitable for transcription” as used herein, may refer to conditions suitable for transcription of DNA to produce RNA. The skilled person would be readily capable of assembling conditions suitable for transcription, for example as described herein in the Materials and Methods. For example, conditions suitable for transcription may comprise a DNA template; an RNA polymerase (such as T7 polymerase); ribonucleotides (rNTPs); a pyrophosphatase and suitable buffer conditions (including, for example, Hepes-KOH, Mg²⁺, DTT, spermidine, etc). In some instances, conditions suitable for transcription and translation comprises a cell lysate (e.g., from an organism of interest) supplemented with a feeding solution comprising an RNA polymerase (such as T7 polymerase); an energy regenerating system (for example, creatine phosphate and creatine kinase); amino acids; ribonucleotides (rNTPs) and other necessary components. In preferred instances, conditions suitable for transcription and translation comprise an in vitro (or cell-free) translation system.

In any of the methods described herein, the incubating is typically performed at physiological temperatures and pH, as described above herein (e.g., about 20° C.-40° C., and about pH 6-8). The incubating is typically performed in a buffered liquid. In some instances, the incubating is performed directly in a cell lysate or an in vitro translation system. For example, the components as described above are added directly to a cell lysate or in vitro translation system and the translation reaction is performed therein.

In some instances, the incubating is performed in the presence of at least one other tRNA, optionally wherein the other tRNA is an endogenous or naturally occurring tRNA. In some instances, the incubating is performed in the presence of a plurality of tRNAs. In some instances, the incubating is performed in the presence of the full complement of natural tRNAs, which includes the tRNA of interest. In some instances, the incubating is performed in the presence of the full complement of natural tRNAs, wherein the full complement of natural tRNAs has the meaning defined herein above. In some instances, the incubating is performed in the presence of a plurality of tRNAs suitable for incorporation of all of the twenty natural amino acids. In some instances, the incubating is performed under conditions suitable for translation, i.e., in the presence of the necessary components for a translation reaction to occur, as described above. Conditions suitable for translation may refer to an in vitro translation system/cell-free translation system.

In some instances, the translation reaction occurs in the presence of the oligonucleotides of the present invention. In some instances, the translation reaction occurs in the presence of the oligonucleotides of the present invention and in the presence of the tRNA of interest. In some instances, the translation reaction occurs in the presence of the oligonucleotides of the present invention and in the presence of the full complement of natural tRNAs, one of which is the tRNA of interest. In some preferred instances, the tRNA of interest is not removed from mixture, i.e., the tRNA of interest is not purified away or pulled-down.

In some preferred instances, the components (a) an mRNA comprising a codon that is recognised by a tRNA of interest; (b) an oligonucleotide of the invention that hybridises to the tRNA of interest when said tRNA is in a folded state, thereby disrupting the function of the tRNA; and (c) a tRNA that (i) recognises the same codon as the tRNA of interest and (ii) is linked to an unnatural amino acid; are incubated simultaneously. In some preferred instances, the components (a) an mRNA comprising a codon that is recognised by a tRNA of interest; (b) an oligonucleotide of the invention that hybridises to the tRNA of interest when said tRNA is in a folded state, thereby disrupting the function of the tRNA; (c) an orthogonal tRNA that recognises the same codon as the tRNA of interest; (d) an unnatural amino acid; and (e) an orthogonal aminoacyl-tRNA synthetase suitable for charging the orthogonal tRNA with the unnatural amino acid; are incubated simultaneously. In some preferred instances, the oligonucleotides of the invention are incubated with the tRNA of interest in the presence of the full complement of natural tRNAs, optionally in a cell lysate, prior to addition of (a) the mRNA; (b) the tRNA that (i) recognises the same codon as the tRNA of interest and (ii) is linked to an unnatural amino acid; or the orthogonal tRNA and the orthogonal aminoacyl-tRNA synthetase; (c) an unnatural amino acid; and (d) under conditions suitable for translation of said mRNA.

Alternatively, in some modified instances of the method, the method comprises in a first step incubating:

(a) an oligonucleotide of the invention that hybridises to the tRNA of interest when said tRNA is in a folded state; and
(b) a full complement of natural tRNAs, which includes the tRNA of interest.
Optionally wherein the full-complement or total tRNA mixture is prepared by purifying the total tRNA from cells or a cell lysate, for example, using phenol extraction or chromatography using an ethanolamine sepharose matrix. The method further comprises in a second step incubating:
(c) the mixture resulting from the first step, of the oligonucleotide of the invention and the full complement of (total) tRNA;
(d) an mRNA comprising a codon that is recognised by a tRNA of interest, as described herein; and either:
(e) a tRNA that (i) recognises the same codon as the tRNA of interest and (ii) is linked to an unnatural amino acid; or
(e) an orthogonal tRNA that recognises the same codon as the tRNA of interest; an unnatural amino acid; and an orthogonal aminoacyl-tRNA synthetase suitable for charging the orthogonal tRNA with the unnatural amino acid;
under conditions suitable for translation of said mRNA, as described herein.

In an alternative embodiment, a method for producing a polypeptide comprising at least one unnatural amino acid comprises in a first step incubating:

(a) an oligonucleotide of the invention that hybridises to the tRNA of interest when said tRNA is in a folded state; and
(b) a full complement of natural tRNAs, which includes the tRNA of interest.
Optionally the full-complement of tRNAs is comprised within a cell lysate or is prepared by purifying the total tRNA from a cell lysate, for example, using phenol extraction or ethanolamine sepharose matrix chromatography.

The above method further comprises in a second step removing the oligonucleotide of the present invention and the tRNA of interest from the other natural tRNAs, i.e., the oligonucleotide and bound (hybridised) tRNA of interest are purified away from or out of the mixture of the remaining natural tRNAs. This may be done, for example, by labelling the oligonucleotide of interest and using an affinity capture method that targets the label. The skilled person is also capable of performing other methods for removing an oligonucleotide and the bound tRNA from the mixture based on their common general knowledge.

The method further comprises in a third step incubating:

(c) the mixture resulting from the first step, comprising the remaining complement of tRNAs that does not include the tRNA of interest;
(d) an mRNA comprising a codon that is recognised by the tRNA of interest, as described herein; and either:
(e) a tRNA that (i) recognises the same codon as the tRNA of interest and (ii) is linked to an unnatural amino acid; or
(e) an orthogonal tRNA that recognises the same codon as the tRNA of interest; an unnatural amino acid; and an orthogonal aminoacyl-tRNA synthetase suitable for charging the orthogonal tRNA with the unnatural amino acid;
under conditions suitable for translation of said mRNA, as described herein.

In some instances, the methods of the present invention may be used to incorporate a plurality of different unnatural amino acids into a polypeptide. For example, the methods of the present invention may be used to produce a polypeptide comprising 1, 2, 3, 4, 5, 6, 7, 8, 9 or 10 different unnatural amino acids. The methods of the present invention may be used to produce a polypeptide comprising at least 2 different unnatural amino acids. The skilled person will appreciate that this could be achieved by using 1, 2, 3, 4, 5, 6, 7, 8, 9 or 10, preferably at least 2, different oligonucleotides of the invention to disrupt the function of 1, 2, 3, 4, 5, 6, 7, 8, 9 or 10, preferably at least 2, different tRNAs of interest. The codons recognised by these tRNAs of interest could then be reassigned to encode 1, 2, 3, 4, 5, 6, 7, 8, 9 or 10, preferably at least 2, different unnatural amino acids, by utilising 1, 2, 3, 4, 5, 6, 7, 8, 9 or 10, preferably at least 2, different tRNAs linked to different unnatural amino acids that recognise different specific codons corresponding to those recognised by each of the tRNAs of interest.

### Compositions and Kits

The present invention also provides a composition or kit comprising:

(a) an oligonucleotide of the present invention that hybridises to a tRNA of interest when said tRNA is in a folded state; and
(b) one or more of:
(i) a tRNA that recognises the same codon as the tRNA of interest and is linked to an unnatural amino acid, as described herein; or (i) an orthogonal tRNA that recognises the same codon as the tRNA of interest, as described herein; an unnatural amino acid, as described herein; and an orthogonal aminoacyl-tRNA synthetase suitable for charging the orthogonal tRNA with the unnatural amino acid, as described herein;
(ii) a plurality of tRNAs, comprising the tRNA of interest, as described herein; and/or
(iii) one or more translation reagents.

In some instances, the plurality of tRNAs may comprise the full complement of endogenous or natural tRNAs, wherein the full complement of tRNAs has the meaning as defined herein. In some instances, the plurality of tRNAs comprises tRNAs suitable for incorporation of all of the twenty natural amino acids. In some instances, the plurality of tRNAs may be comprised in a cell lysate.

The skilled person would be capable of identifying the one or more translation reagents. The composition or kit may comprise one or more of a buffered liquid, such as HEPES or Tris buffer; an mRNA (as described herein); a plurality of aminoacyl-tRNA synthetases; any or all of the twenty naturally occurring amino acids; ribosomes; and/or the necessary protein factors for translation (e.g., initiation factors (IF1, IF2, and IF3); elongation factors (EF-Tu, EF-G); release factors (RF1, RF2, RF-3)), T7 polymerase for transcription, rNTPs (rATP, rGTP, rCTP, rUTP), Mg²⁺, K⁺, DTT, PEG, folic acid, acetyl phosphate, energy generating system (for example, creatine phosphate/creatine phosphokinase, components of glycolysis or oxidative phosphorylation system), protease inhibitor. In some instance, the composition or kit comprises a cell lysate; a cell lysate and a feeding solution (for example, comprising one or more of T7 polymerase for transcription, rNTPs (rATP, rGTP, rCTP, rUTP), Mg²⁺, K⁺, DTT, PEG, folic acid, acetyl phosphate, energy generating system (for example, creatine phosphate/creatine phosphokinase, components of glycolysis or oxidative phosphorylation system), amino acids, protease inhibitor) for assembling conditions suitable for translation and optionally transcription; and/or an in vitro translation system.

The composition may comprise a cell lysate comprising one or more factors providing for translation. The lysate may comprise ribosomes, protein factors for translation and a complement of tRNAs comprising the tRNA of interest. The composition may comprise an in vitro translation system. The skilled person would be capable of assembling a cell lysate and a feeding solution, as described herein, to produce an in vitro translation system using methods well known in the art. The cell lysate can be prepared from the cells of any selected organism of interest. Preferably the cell lysate is prepared from human cells, mammalian cells, bacterial cells, trypanosome cells or yeast cells. In preferred instances the cell lysate is prepared from the bacteria Escherichia coli or the trypanosome Leishmania tarentolae. The in vitro translation system can be prepared using components isolated from any selected organism of interest. Preferably the in vitro translation system is prepared using components from mammalian cells, bacteria, trypanosomes or yeast. In preferred instances the in vitro translation system is prepared using components from the bacteria Escherichia coli or the trypanosome Leishmania tarentolae.

### Cell-Based Methods for Synthesising Proteins Comprising Unnatural Amino Acids

The present invention provides a vector suitable for expressing an oligonucleotide of the present invention and cell comprising such a vector. Thus, the present invention also provides a cell comprising (and typically expressing) an oligonucleotide of the present invention.

The vector may be suitable for prokaryotic or eukaryotic expression of the oligonucleotide. The vector may be a plasmid. The vector may be comprised within a virus. The vector may be integrated, optionally stably integrated, into the genome of a host cell. The vector may not be integrated into the genome of a host cell. The vector may comprise a marker gene. The vector may comprise a selectable marker gene. The vector may comprise an inducible promoter, optionally a chemically-regulated or physically-regulated promoter. Chemically-regulated promoters include those whose transcriptional activity is regulated by the presence or absence of a chemical such as small molecules (e.g., IPTG, arabinose, alcohol, tetracycline), steroids, metals and other compounds. Physically-regulated promoters include those whose transcriptional activity is regulated by changes in a physical stimulus, such as heat, light, pH, salts, osmotic pressure. The expression of the oligonucleotide of the present invention may be under the control of the inducible promoter. Inducible production of the oligonucleotides may be useful to avoid lethal effect.

The vector may be introduced into a cell for expression of the oligonucleotide using any suitable method known in the art. For example, the vector may be introduced into eukaryotic cells by virus-mediated gene transfer, calcium phosphate, electroporation, cell squeezing, cationic polymers (e.g., PEI), lipofection (i.e., liposome transfection), heat shock, fugene, sonoporation, lithium acetate, polyethylene glycol, single-stranded DNA and/or particle bombardment. For example, the vector may be introduced into prokaryotic cells by bacteriophage- or virus-mediated gene transfer, divalent cations (e.g., calcium chloride), cold shock, heat shock, electroporation. The cell may be eukaryotic or prokaryotic. The cell may be a yeast cell, preferably Saccharomyces cerevisiae or Schizosaccharomyces pombe, a bacterial cell, preferably Escherichia coli, or a trypanosome, preferably Leishmania tarentolae. Preferably, the cell is a bacterial cell and is Escherichia coli.

In some instances, the cell further comprises one or more of:

(a) an mRNA comprising a codon that is recognised by a tRNA of interest, as described herein;
(b) an orthogonal tRNA that recognises the same codon as the tRNA of interest, as described herein;
(c) an unnatural amino acid, as described herein; and/or
(d) an orthogonal aminoacyl-tRNA synthetase suitable for charging the orthogonal tRNA with the unnatural amino acid, as described herein.

In some instances, the unnatural amino acid is not capable of being expressed by a cell and is added exogenously to the cells. In some instances the cell further expresses an mRNA comprising a codon that is recognised by a tRNA of interest, as described herein. In some instances the cell further expresses an orthogonal tRNA that recognises the same codon as the tRNA of interest, as described herein. In some instances the cell further expresses or produces an unnatural amino acid, as described herein. In some instances the cell further expresses an orthogonal aminoacyl-tRNA synthetase suitable for charging the orthogonal tRNA with the unnatural amino acid, as describe herein. In some instances the cell further expresses an mRNA comprising a codon that is recognised by a tRNA of interest, as described herein; an orthogonal tRNA that recognises the same codon as the tRNA of interest, as described herein and an orthogonal aminoacyl-tRNA synthetase suitable for charging the orthogonal tRNA with the unnatural amino acid, as described herein. In such instances, an unnatural amino acid may be added to or provided to the cells. In some instances the cell further expresses an mRNA comprising a codon that is recognised by a tRNA of interest, as described herein; an orthogonal tRNA that recognises the same codon as the tRNA of interest, as described herein; an unnatural amino acid, as described herein; and an orthogonal aminoacyl-tRNA synthetase suitable for charging the orthogonal tRNA with the unnatural amino acid, as described herein.

In some instances the cell produces a polypeptide comprising at least one unnatural amino acid, as described herein. In some instances the polypeptide is secreted by the cell, for example into the cell growth media. In some instances the polypeptide may be purified from the cell growth media or from the cell lysate.

In some instances, a lysate may be prepared from the cells described above, which comprises any of the above components comprised in the cell as described above. The lysate typically comprises an oligonucleotide of the invention.

In some instances the oligonucleotide of the invention may be added to a cell lysate prepared from cells expressing other components of the translation reaction. For example, in some instances cells are provided which express or comprise one or more, preferably all, of:
(a) an mRNA comprising a codon that is recognised by a tRNA of interest, as described herein;
(b) an orthogonal tRNA that recognises the same codon as the tRNA of interest, as described herein;
(c) an unnatural amino acid, as described herein; and/or
(d) an orthogonal aminoacyl-tRNA synthetase suitable for charging the orthogonal tRNA with the unnatural amino acid, as described herein. A cell lysate may be prepared from such cells and an oligonucleotide of the invention added to the cell lysate in vitro. Thus, in some instances, the present invention provides a composition comprising a lysate prepared from a cell comprising one or more, preferably all, of:
(a) an mRNA comprising a codon that is recognised by a tRNA of interest;
(b) an orthogonal tRNA that recognises the same codon as the tRNA of interest;
(c) an unnatural amino acid; and/or
(d) an orthogonal aminoacyl-tRNA synthetase suitable for charging the orthogonal tRNA with the unnatural amino acid;
the composition further comprising an oligonucleotide of the invention.

In some instances the composition may comprise a lysate prepared from a cell comprising one or more, preferably all, of (a) an mRNA comprising a codon that is recognised by a tRNA of interest; (b) an orthogonal tRNA that recognises the same codon as the tRNA of interest; and/or (c) an orthogonal aminoacyl-tRNA synthetase suitable for charging the orthogonal tRNA with the unnatural amino acid; wherein the composition further comprises an unnatural amino acid and an oligonucleotide of the invention.

The present invention also provides the use of a lysate prepared from the cells described herein for use in producing a polypeptide comprising at least one unnatural amino acid, optionally according to the methods of the present invention, as described herein.

### EXAMPLES

### Materials and Methods

### Materials

The sequences of the oligonucleotides used in this work are provided in Table 1. The oligonucleotides were synthesized by Integrated DNA Technologies. The p-azido-L-phenylalanine (AzF) and n-propargyl-L-lysine (PrK) were purchased from SynChem and Sirius Fine Chemicals, respectively. Ethanolamine-sepharose used for tRNA purification was prepared by coupling ethanolamine to epoxy-activated Sepharose 6B (GE Healthcare) as described (5).

TABLE 1

| Name | Sequence | Numbering |
|---|---|---|
| M1 | /5Cy3/dCdTdTdTdTdGdAdCdCdGdCdAdTdAdCdTdCdCdCdTd | N34-N47j |
| | TdAdGdC (SEQ ID NO: 1) | |
| M2 | /5Cy3/rCrUrUrUrUrGrArCrCrGrCrArUrArCrUrCrCrCrUrUrArG | |
| | rC (SEQ ID NO: 2) | |
| M3 | /5Cy3/mCmUmUmUdTdGdAdCdCdGdCdAdTdAdCdTdCdCdCd | |
| | TmUmAmGmC (SEQ ID NO: 3) | |
| M4 | /5Cy3/mCmUmUmUrUrGrArCrCrGrCrArUrArCrUrCrCrCrUmU | |
| | mAmGmC (SEQ ID NO: 4) | |
| M5 | /5Cy3/mCmUmUmUmUmGmAmCmCmGmCmAmUmAmCmU | |
| | mCmCmCmUmUmAmGmC (SEQ ID NO: 5) | |
| M6 | /5Cy3/dAdGdCdAdGdGdGdGdAdGdCdGdCdCdTdTdCdAdGdC | N8-N36 |
| | dCdTdCdTdCdGdGdCdCdA (SEQ ID NO: 6) | |
| M7 | /5Cy3/rArGrCrArGrGrGrGrArGrCrGrCrCrUrUrCrArGrCrCrUrC | |
| | rUrCrGrGrCrCrA (SEQ ID NO: 7) | |
| M8 | /5Cy3/mAmGmCmAmGmGmGmGmAmGmCmGmCmCmUmU | |
| | mCmAmGmCmCmUmCmUmCmGmGmCmCmA (SEQ ID NO: 8) | |
| M5-1 | mCmUmUmUmUmGmAmCmCmGmCmAmUmAmCmUmCmC | N34-N47j |
| | mCmUmUmAmGmC (SEQ ID NO: 9) | |
| M5-2 | mCmUmUmUmUmGmArCrCmGmCrAmUmArCmUmCmCmCr | N34-N47j |
| | UrUmAmGmC (SEQ ID NO: 10) | |
| M5-3 | mCmUmUmUrUmGmArCrCrGmCrArUrArCmUrCmCrCrUrUm | N34-N47j |
| | AmGrC (SEQ ID NO: 11) | |
| M5T1 | mUmUmUmGmAmCmCmGmCmAmUmAmCmUmCmCmCmU | N34-N47h |
| | mUmAmGmC/3AmMO/ (SEQ ID NO: 12) | |
| M5T2 | mGmAmCmCmGmCmAmUmAmCmUmCmCmCmUmUmAmG | N34-N47e |
| | mC/3AmMO/ (SEQ ID NO: 13) | |
| M5T3 | mCmUmUmUmUmGmAmCmCmGmCmAmUmAmCmUmCmC | N38-N47j |
| | mCmU/3AmMO/ (SEQ ID NO: 14) | |
| M5T4 | mCmUmUmUmUmGmAmCmCmGmCmAmU/3AmMO/ | N45-N47j |
| | (SEQ ID NO: 15) | |
| R1 | mAmGmGmAmGmGmGmGmCmUmCmGmUmUmAmUmAm | N13-N36 |
| | UmCmCmAmUmUmU (SEQ ID NO: 17) | |
| R2 | mCmCmUmGmCmAmAmUmUmAmGmCmCmCmUmUmAmG | N31-N53 |
| | mGmAmGmG (SEQ ID NO: 18) | |
| R3 | mAmUmCmGmAmAmCmCmUmGmCmAmAmUmUmAmGmC | N34-N59 |
| | mCmCmUmUmAmGmG (SEQ ID NO: 19) | |
| R4 | mUmGmGmUmGmUmCmCmCmCmUmGmCmAmGmGmAmA | N53-N76 |
| | mUmCmGmAmAmC (SEQ ID NO: 20) | |
| L1 | /5Cy3/mUmUmAmGmCmAmGmGmCmAmGmGmCmGmCmC | N13-N38 |
| | mUmUmAmAmCmCmAmCmUmC (SEQ ID NO: 21) | |
| L2 | /5Cy3/mGmAmGmAmUmCmAmCmAmCmCmUmGmCmUmU | N32-N47e |
| | mAmGmCmAmG (SEQ ID NO: 22) | |
| L3 | mGmCmUmUmAmGmCmAmGmGmCmAmGmGmCmGmCmC | N21-N40 |
| | mUmU (SEQ ID NO: 23) | |
| L4 | mUmGmGmCmGmCmAmAmAmCmGmGmAmAmGmGmGmU | N56-N76 |
| | mUmCmG (SEQ ID NO: 24) | |
| L5 | mUmUmAmAmCmCmAmCmUmCmGmGmCmCmAmCmAmU | N1-N21 |
| | mUmUmGmC (SEQ ID NO: 25) | |
| L6 | mUmUmCmGmAmAmCmCmUmUmCmGmCmGmUmGmAmG | N47-N59 |
| | mAmUmC (SEQ ID NO: 26) | |

dN:deoxyribonucleotides; rN: ribonucleotides; mN: 2′-O-methyl ribonucleotides

Microscale Thermophoresis (MST) Analysis of tRNA Interaction with Oligonucleotides

The equilibrium dissociation constants between antisense oligonucleotides and tRNA^{Ser}GCU were evaluated using Monolith NT.115 from NanoTemper following the manufacturer's protocol. Briefly, the unlabeled tRNA molecules were prepared at 8 μM concentration and serially diluted 1 to 4 to the lowest concentration of 0.03 nM in buffer A (20 mM Tris-HCl, pH 7.5, 10 mM MgCl₂, 100 mM NaCl, 0.05 mg/ml BSA, 0.05% Tween 20, 2% PEG8000). The equal volume of Cy3-labelled antisense oligonucleotides in the same buffer were added in tRNA sample to bring their final concentration to 10 nM. After incubation at room temperature for 3 hours, the mixtures were transferred into the NT.115 standard treated capillaries (NanoTemper Technologies) and fluorescently scanned with both LED and MST power set at 80%. The results were then analyzed using Monolith software and K_{d} value was calculated using nonlinear fit law of mass action equation. The same procedure was used to measure the binding affinities between antisense oligonucleotides M5-1 and Cy3-labelled tRNA with the concentration of the former varied from 0.008 to 125 nM while the latter kept at 5 nM concentration.

### Gel Shift Assay

Modified oligonucleotides (M1-M8) at the final concentration of 0.5 μM were incubated with 2.5 μM of indicated tRNA in a total reaction volume of 10 μl of TMN buffer (20 mM Tris-HCl, pH7.5, 10 mM MgCl₂, 100 mM NaCl) (25) at 37° C. for 15 min. The reaction was then chilled on ice and diluted with the equal amount of cold 2×RNA loading dye (95% formamide, 0.0025% bromphenol blue). Samples in 5 μl aliquots were loaded onto 8% denaturing PAGE gel containing TBE, 6 M Urea and 2.5 mM MgCl₂. The gel electrophoresis was performed in 1×TBE buffer containing 2.5 mM MgCl₂ at 70V for 10 mM followed by the voltage increase to 120V until the dye reached the end of the gel. After electrophoresis, the gel was stained with SYBR Green II dye for 15 min and subjected to scanning using Gel Doc XR Imaging System (Bio-Rad).

tRNA Labelling

tRNA labelling was performed by oxidation of its 3′ terminus with sodium periodate and subsequent reaction with hydrazide-Cy3. Briefly, 15 μM tRNA and 100 mM NaOAc (pH 5) in a total volume of 1 ml were mixed and placed on ice for 5 min. After adding NaIO₄ to 3 mM concentration, the oxidation reaction was performed in the dark for 30 min. The tRNA was then precipitated with ethanol and washed once with 70% ethanol. After re-suspending the pellet to 0.5 mM final concentration in the solution of 0.1 M NaOAc (pH 5.0), the tRNA was then supplemented with 5 mM Cy3-hydrazide which was dissolved in DMSO and incubated with agitation in the dark at room temperature for 1 h. After diluting the sample 10 times with 0.1 M NaOAc, tRNA was then precipitated by ethanol. The labelled tRNA was then purified on the POROS® R1 10 μm column (Applied Biosystems) using buffers A and B both containing 0.1 M triethylamine acetate (pH 5.2) and either 1% (Buffer B) or 90% (Buffer C) of acetonitrile using 1-20% linear gradient in 9 minutes run. After HPLC purification, the labelled tRNA fraction was precipitated by ethanol twice, re-suspended in water and stored at −80° C.

### Expression Vector Design and Construction

All expression vectors were based on pLTE vector (GenBank number of KJ541667.1). The DNA templates coding for eGFP ORF with biased codon usages were designed to test reassignment of sense codons. The eGFP ORF containing two consecutive AGC codons (2AGC codon template) was employed to evaluate the levels of functional tRNA^{Ser}GCU in the in vitro translation reaction. A species-independent translational leader sequence (SITS) was used to allow efficient protein synthesis in both prokaryotic and eukaryotic cell-free translation systems. The SITS region includes both the 5′ untranslated region (5′UTR) promoting formation of the initiation complex and the short 3′-part comprising 1AGT codon and coding for the 17 amino acid leader peptide. Two consecutive AGC-codons were placed at the positions 21 and 22 of the resulting protein while the rest of Ser codons were TCCs.

The templates termed 1AGC and 1AGT were constructed for installation of an unnatural amino acid (uAA) at the unique AGC or AGT codon positions. The SITS sequence in these templates was optimized to exclude AGT codon while maintaining the hairpin structure of the leader required for effective translational initiation. For that, two nucleotides in the loop region of the third hairpin were altered from “GUAA” to “UUAG”, thus replacing the AGT codon with GGT while maintaining the presumable stem-loop structure. We experimentally confirmed that this modified SITS sequence could efficiently support protein translation. The single AGC or AGT codon was placed at the position 21 in the ORF. A template denoted as 1AGG codon template was constructed using the modified SITS to evaluate the inactivation level of tRNA^{Arg}CCU in the E. coli in vitro translation system. The single AGG codon was placed at the position 21 in the protein ORF while the rest of arginine were encoded with CGG codons.

The genes with desired sequences containing cloning overhangs compatible with double digested pLTE plasmid were synthesized by IDT. Assembly of the digested plasmid and genes were achieved using Gibson Assembly® Master Mix (NEB). The AGT- or AGG-codon mutations in the eGFP coding sequence were encoded into the primers designed by NEBaseChanger™ online primer design software and introduced using the Q5® Site-Directed Mutagenesis Kit (NEB). The plasmids were verified by sequencing and purified by plasmid Midi kit (QIAGEN) for the use as templates for in vitro translation.

E. coli In Vitro Protein Translation Assay

The E. coli S30 cell extract was prepared from BL21-Gold(DE3) strain using the method as described in (14). For inactivation of selected tRNAs, the S30 cell extract was incubated with antisense oligonucleotides at indicated concentration at 37° C. for 5 minutes and, cooled down on ice prior to assembly of the in vitro translation. The antisense oligonucleotides were added in a volume not exceeding 1/10^{th} of the volume of lysate to minimize the dilution effect on translational efficiency.

The DNA templates coding for eGFP ORFs with biased codon usage were designed and constructed as described above and their sequences are provided in Table 2. The cell-free translation reactions for eGFP production were performed following the previously developed but slightly modified protocol (15). Briefly, each 10 μl reaction consisted of 3.57 μl S30 lysate (parental or treated with antisense oligonucleotide), 10 mM Mg(OAc)₂, 2 mM DTT, 2% PEG 8000, 87 mM Hepes-KOH pH 7.4, 1× protease inhibitor (Complete™ EDTA-free, Roche), 0.1 mg/ml folic acid, 1 mM rNTP mix, 15 mM acetyl phosphate, 25 mM creatine phosphate, 0.6 mM of each amino acid, 1 mM RCWDME amino acid cocktail, 0.05 mg/ml T7 RNA polymerase, 45 U/ml creatine phosphokinase, 50-100 ng/ml DNA Template, in the presence or absence of suppressor tRNA indicated in each experiment. The eGFP production was monitored on a fluorescence spectrometer (Synergy) at 37° C. for 3-5 h using 485 nm excitation and 528 nm emission wavelengths.

TABLE 2

| | No. Ser/Arg | |
|---|---|---|
| | codons in | |
| Name | ORF | ORF sequence |
| 2AGC | 2AGC, | ATGACAGTAATGTATAAAGTCTGTAAAGACATTAAACACG |
| codon | 1AGT, | TAAGTGAAACCATGGAGATCagcagcAAGGGCGAGGAGCTGT |
| template | 10TCC (Ser) | TCACCGGGGTGGTGCCCATCCTGGTCGAGCTGGACGGCGA |
| | | CGTAAACGGCCACAAGTTCtccGTGTCCGGCGAGGGCGAGG |
| | | GCGATGCCACCTACGGCAAGCTGACCCTGAAGTTCATCTG |
| | | CACCACCGGCAAGCTGCCCGTGCCCTGGCCCACCCTCGTG |
| | | ACCACCCTGACCTACGGCGTGCAGTGCTTCtccCGCTACCCC |
| | | GACCACATGAAGCAGCACGACTTCTTCAAGTCCGCCATGC |
| | | CCGAAGGCTACGTCCAGGAGCGCACCATCTTCTTCAAGGA |
| | | CGACGGCAACTACAAGACCCGCGCCGAGGTGAAGTTCGAG |
| | | GGCGACACCCTGGTGAACCGCATCGAGCTGAAGGGCATCG |
| | | ACTTCAAGGAGGACGGCAACATCCTGGGGCACAAGCTGGA |
| | | GTACAACTACAACtccCACAACGTCTATATCATGGCCGACA |
| | | AGCAGAAGAACGGCATCAAGGTGAACTTCAAGATCCGCCA |
| | | CAACATCGAGGACGGCtccGTGCAGCTCGCCGACCACTACC |
| | | AGCAGAACACCCCCATCGGCGACGGCCCCGTGCTGCTGCC |
| | | CGACAACCACTACCTGtccACCCAGTCCGCCCTGtccAAAGAC |
| | | CCCAACGAGAAGCGCGATCACATGGTCCTGCTGGAGTTCG |
| | | TGACCGCCGCCGGGATCACTCTCGGCATGGACGAGCTATA |
| | | CAAGGAGCAGAAGCTGATCtccGAGGAGGATCTGTAA (SEQ |
| | | ID NO: 54) |
| 1AGT | 1AGT, | ATGACAGTAATGTATAAAGTCTGTAAAGACATTAAACACT |
| codon | 10TCG (Ser) | TAGGTGAAACCATGGAGATCagtAAGGGCGAGGAGCTGTTC |
| template | | ACCGGGGTGGTGCCCATCCTGGTCGAGCTGGACGGCGACG |
| | | TAAACGGCCACAAGTTCtcgGTGtcgGGCGAGGGCGAGGGCG |
| | | ATGCCACCTACGGCAAGCTGACCCTGAAGTTCATCTGCAC |
| | | CACCGGCAAGCTGCCCGTGCCCTGGCCCACCCTGGTGACC |
| | | ACCCTGACCTACGGCGTGCAGTGCTTCtcgCGGTACCCCGAC |
| | | CACATGAAGCAGCACGACTTCTTCAAGtcgGCCATGCCCGA |
| | | AGGCTACGTCCAGGAGCGGACCATCTTCTTCAAGGACGAC |
| | | GGCAACTACAAGACCCGGGCCGAGGTGAAGTTCGAGGGC |
| | | GACACCCTGGTGAACCGGATCGAGCTGAAGGGCATCGACT |
| | | TCAAGGAGGACGGCAACATCCTGGGGCACAAGCTGGAGTA |
| | | CAACTACAACtcgCACAACGTCTATATCATGGCCGACAAGC |
| | | AGAAGAACGGCATCAAGGTGAACTTCAAGATCCGGCACAA |
| | | CATCGAGGACGGCtcgGTGCAGCTGGCCGACCACTACCAGC |
| | | AGAACACCCCCATCGGCGACGGCCCCGTGCTGCTGCCCGA |
| | | CAACCACTACCTGtcgACCCAGtcgGCCCTGtcgAAAGACCCCA |
| | | ACGAGAAGCGGGATCACATGGTCCTGCTGGAGTTCGTGAC |
| | | CGCCGCCGGGATCACTCTGGGCATGGACGAGCTATACAAG |
| | | GAGCAGAAGCTGATCtcgGAGGAGGATCTGTAA (SEQ ID |
| | | NO: 64) |
| 1AGC | 1AGC, | ATGACAGTAATGTATAAAGTCTGTAAAGACATTAAACACT |
| codon | 3TCA, 7TCG | TAGGTGAAACCATGGAGATCagcAAGGGAGAGGAGCTGTTC |
| template | (Ser) | ACAGGAGTGGTGCCGATCCTGGTAGAGCTGGACGGAGACG |
| | | TAAACGGGCACAAGTTCtcgGTGtcaGGAGAGGGAGAGGGAG |
| | | ATGCCACGTACGGGAAGCTGACACTGAAGTTCATCTGCAC |
| | | GACAGGAAAGCTGCCAGTACCGTGGCCGACGCTGGTGACG |
| | | ACACTGACGTACGGAGTACAGTGCTTCtcaCGGTACCCAGA |
| | | CCACATGAAGCAGCACGACTTCTTCAAGtcgGCTATGCCAG |
| | | AAGGGTACGTACAGGAGAGAACGATCTTCTTCAAGGACGA |
| | | CGGAAACTACAAGACACGGGCTGAGGTGAAGTTCGAGGG |
| | | AGACACGCTGGTGAACAGGATCGAGCTGAAGGGAATCGA |
| | | CTTCAAGGAGGACGGAAACATCCTGGGACACAAGCTGGAG |
| | | TACAACTACAACtcgCACAACGTATATATCATGGCCGACAA |
| | | GCAGAAGAACGGGATCAAGGTGAACTTCAAGATCCGGCAC |
| | | AACATCGAGGACGGAtcgGTACAGCTGGCTGACCACTACCA |
| | | GCAGAACACACCGATCGGAGACGGACCAGTACTGCTGCCA |
| | | GACAACCACTACCTGtcgACGCAGtcaGCTCTGtcgAAAGACCC |
| | | GAACGAGAAGAGAGATCACATGGTACTGCTGGAGTTCGTG |
| | | ACAGCCGCTGGAATCACGCTGGGGATGGACGAGCTGTACA |
| | | AGGAGCAGAAGCTGATCtcgGAGGAGGATCTGTAA (SEQ ID |
| | | NO: 60) |
| 1AGG | 1AGG, | ATGACAGTAATGTATAAAGTCTGTAAAGACATTAAACACT |
| codon | 6CGG (Arg) | TAGGTGAAACCATGGAGATCaggAAGGGCGAGGAGCTGTTC |
| template | | ACCGGGGTGGTGCCCATCCTGGTCGAGCTGGACGGCGACG |
| | | TAAACGGCCACAAGTTCTCGGTGTCGGGCGAGGGCGAGGG |
| | | CGATGCCACCTACGGCAAGCTGACCCTGAAGTTCATCTGC |
| | | ACCACCGGCAAGCTGCCCGTGCCCTGGCCCACCCTGGTGA |
| | | CCACCCTGACCTACGGCGTGCAGTGCTTCTCGcggTACCCCG |
| | | ACCACATGAAGCAGCACGACTTCTTCAAGTCGGCCATGCC |
| | | CGAAGGCTACGTCCAGGAGcggACCATCTTCTTCAAGGACG |
| | | ACGGCAACTACAAGACCcggGCCGAGGTGAAGTTCGAGGG |
| | | CGACACCCTGGTGAACcggATCGAGCTGAAGGGCATCGACT |
| | | TCAAGGAGGACGGCAACATCCTGGGGCACAAGCTGGAGTA |
| | | CAACTACAACTCGCACAACGTCTATATCATGGCCGACAAG |
| | | CAGAAGAACGGCATCAAGGTGAACTTCAAGATCcggCACAA |
| | | CATCGAGGACGGCTCGGTGCAGCTGGCCGACCACTACCAG |
| | | CAGAACACCCCCATCGGCGACGGCCCCGTGCTGCTGCCCG |
| | | ACAACCACTACCTGTCGACCCAGTCGGCCCTGTCGAAAGA |
| | | CCCCAACGAGAAGcggGATCACATGGTCCTGCTGGAGTTCG |
| | | TGACCGCCGCCGGGATCACTCTGGGCATGGACGAGCTATA |
| | | CAAGGAGCAGAAGCTGATCTCGGAGGAGGATCTGTAA |
| | | (SEQ ID NO: 68) |

Production of Synthetic tRNAs and Orthogonal Aminoacyl-tRNA Synthetases:

The sequences of synthetic (or orthogonal) tRNAs used in this work are provided in Table 3. These synthetic tRNAs were prepared and purified as described before (22). Briefly, the DNA template with 5′-T7 promoter and tRNA coding sequences were constructed by overlap PCR. The resulting products were purified by ethanol precipitation and then dissolved in water for run-off transcription using T7 RNA polymerase. The transcription reactions were performed in 40 mM Hepes-KOH (pH 7.9), 18 mM Mg(OAc)₂, 2 mM spermidine, 40 mM DTT, 5 mM each rNTP, 0.25 U/ml yeast inorganic pyrophosphatase, 10 μg/mL T7 RNA polymerase and 0.25 μM DNA template at 32° C. for 3 h. The tRNA transcripts were purified by affinity chromatography using ethanolamine-Sepharose matrix (5).

Two orthogonal tRNA synthetases including PylRSAF and AzFRS were expressed in E. coli and purified by Ni²⁺ affinity chromatography and gel filtration as described previously (5).

TABLE 3
Synthetic tRNAs Sequence

| Ec tRNA^{Gly}GCU | GCGGGCAUCGUAUAAUGGCUAUUACCUCAGCCUgcuAAGCU |
|---|---|
| | GAUGAUGCGGGUUCGAUUCCCGCUGCCCGCUCCA (SEQ ID |
| | NO: 45) |
| Ec tRNA^{Arg}CCU | GUCCUCUUAGUUAAAUGGAUAUAACGAGCCCCUccuAAGGG |
| | CUAAUUGCAGGUUCGAUUCCUGCAGGGGACACCA (SEQ ID |
| | NO: 36) |
| Lt tRNA^{Gly}GCU | GCGCCGCUGGUCUAGUGGCAUGAUGGUACCCUgcuAAGGUA |
| | UUGACCCGGGUUCGAUUCCCGGGCGGCGCACCA (SEQ ID |
| | NO: 46) |
| tRNA^{AzF}GCU | CCGGCCGUAGUUCAGCAGGGCAGAACGGCGGACUgcuAAUC |
| | CGCAUGGCAUGGGUUCAAAUCCCAUCGGCCGGACCA (SEQ |
| | ID NO: 48) |
| tRNA^{AzF}ACU | CCGGCCGUAGUUCAGCAGGGCAGAACGGCGGACUacuAAUC |
| | CGCAUGGCAUGGGUUCAAAUCCCAUCGGCCGGACCA (SEQ |
| | ID NO: 47) |
| tRNA^{Pyl}GCU | GGAAACCUGAUCAUGUAGAUCGAACGGACUgcuAAUCCGU |
| | UCAGCCGGGUUAGAUUCCCGGGGUUUCCGCCA (SEQ ID NO: |
| | 50) |
| tRNA^{Pyl}ACU | GGAAACCUGAUCAUGUAGAUCGAACGGACUacuAAUCCGU |
| | UCAGCCGGGUUAGAUUCCCGGGGUUUCCGCCA (SEQ ID NO: |
| | 49) |

### Mass Spectrometry

In vitro expressed eGFP proteins were incubated with anti-GFP VHH-Sepharose matrix (26) at room temperature for 30 min. The matrix was washed 3 times with PBS buffer containing 0.1% Triton and once with PBS buffer. The protein was eluted with SDS PAGE loading buffer at 98° C. for 5 min and separated on NuPAGE 4-12% Bis-Tris Gel (Thermo Fisher Scientific). The corresponding band was cut out for digestion with sequencing grade trypsin following the in gel tryptic digestion protocol from Thermo Scientific. The supernatant containing the peptides was lyophilized for at least 3 hrs. The samples were dissolved in 20 μL of 0.1% formic acid before LC-MS/MS analysis using a Shimadzu Nexera uHPLC interfaced with a 5600 TripleTOF with a duo electrospray ion source (AB SCIEX). The 10 μl sample was injected onto a Zorbax C18 1.8 μm column (Agilent) using buffers C and D both containing 0.1% formic acid and either none (Buffer D) or 90% (Buffer E) of acetonitrile. The program using 1-40% linear gradient over 50 minutes run was applied. The ion spray voltage was set to 5500V. Full scan of TOF MS data was acquired over the mass range of 300-2000 Da and for product ion ms/ms 100-1800. Ions observed in the TOF-MS scan exceeding a threshold of 100 counts and a charge state of +2 to +5 were set to trigger the acquisition of product ion, ms/ms spectra of the resultant 10 most intense ions. The data was acquired and analyzed using Analyst TF 1.6 software (AB SCIEX) to assess the identity of the amino acid incorporated at the desired position.

Reconstruction of Specific tRNA-Dependent LTE Translation System

The L. tarentolae extract (LTE) was prepared as described before (27) and stored at −80° C. in buffer F containing 45 mM Hepes-KOH pH 7.6, 10 mM KOAc, 10.5 mM Mg(OAc)₂ and 0.5 mM rGTP for tRNA depletion. Typically 2.5 ml of LTE was re-buffered by loading it on NAP-25 column (GE healthcare) equilibrated with buffer G of 25 mM KCl, 10 mM NaCl, 1.1 mM Mg(OAc)₂, 0.1 mM EDTA, 10 mM Hepes-KOH (pH7.5) and 100 mM KOAc. After re-buffering the lysate was incubated with 0.8 ml of settled ethanolamine-Sepharose matrix at 4° C. for 30 min with orbital shaking. After incubation the supernatant was collected. The matrix was then washed with 1 ml of buffer G containing 180 mM KOAc. The flow-through was combined with the supernatant from the previous step, snap frozen and stored at −80° C.

Total tRNA mixture was prepared from L. tarentolae cell culture using phenol extraction by modified procedure of Zubay's method (28). In brief, the L. tarentolae culture was inoculated into 1 L of TB medium (Tryptone 12 g/L, Yeast extract 24 g/L, Glycerol 8 ml/L, Glucose 1 g/L, KH₂PO₄ 2.31 g/L, K₂HPO₄ 2.54 g/L) and grown at 26° C. until OD₆₀₀ reached 3-3.5 (1.0-1.3×10⁸ cells/ml). The cells were pelleted by centrifugation at 2500×g and washed with SEB buffer (45 mM Hepes-KOH pH 7.6, 250 mM Sucrose, 10 mM KOAc, 14 mM Mg(OAc)₂, 0.5 mM rGTP). The nucleic acids were then extracted twice from the pelleted cells with 88% redistilled phenol, and precipitated by addition of 0.05 volume of 4 M KOAc and double volume of absolute ethanol. The tRNA precipitate was re-extracted with 1M NaCl twice, the supernatant was ethanol-precipitated twice. The tRNA precipitate was dissolved in the nuclease-free water.

The specific tRNA species in the total L. tarentolae tRNA mixture were inactivated using antisense oligos where their sequences are provided in Table 1. The isolated total tRNA at 3 μg/μl final concentration was mixed with antisense oligonucleotides at 60 μM concentration in water and incubated at 37° C. for 30 min. The translation reaction was set up using modified standard conditions (27). The reaction (10 μl) contained 50% tRNA-depleted LTE (5 μl), 1.67 μl of 3 μg/μl of antisense-oligonucleotide treated total tRNA, 4.45 mM Mg(OAc)₂, 0.24 mM spermidine, 2 mM DTT, 40 mM creatine phosphate, 20 mM Hepes-KOH pH (7.6), 1% (v/v) PEG 3000, lx protease inhibitor (Complete™ EDTA-free, Roche), 0.14 mM of each amino acid, 1.5 mM rNTP mix (ATP, GTP, UTP and CTP), 0.01 mM anti-splice leader DNA oligonucleotide, 0.1 mg/ml T7 RNA polymerase, 40 U/ml creatine phosphokinase), 50-100 ng/μl DNA Template, with or without 20 μM AGC-suppressor tRNA (L. tarentolae tRNA^{Gly}GCU) as indicated.

For inactivation of selected tRNAs in LTE, the cell extract was incubated with antisense oligonucleotides at 30 μM concentration, at 37° C. for 5 minutes and then cooled down on ice prior to assembly of the in vitro translation. The reaction (10 μl) contained 50% antisense oligonucleotide-treated LTE (5 μl), Mg(OAc)₂ at 3.5 mM and other components as described above for the reconstituted system. The progress of translation was monitored on the Synergy plate reader at 27° C. for 3-5 h using λₑₓ485/λₑₘ528.

### Example 1

Antisense Oligonucleotides can Selectively and Efficiently Sequester their tRNA Targets

Inactivation of a specific tRNA in the context of in vitro translation system is a challenging task as the size, shape and physical stability of tRNAs are similar. Most of the unique primary sequence determinants are embedded in the secondary and tertiary structures and have low solvent accessibility. The unpaired single-stranded segments of the anticodon loop and the 3′-CCA end are short and share significant similarity among tRNAs. Thus, we set out to devise a generally applicable approach for selective tRNA inactivation by exploiting the isoacceptor-specific differences in the primary structure.

Antisense oligonucleotides have been extensively used in vivo and in vitro to target eukaryotic or bacterial RNAs by promoting their cleavage or impairing their interactions with the cellular machinery (29, 30). With introduction of novel oligonucleotide chemistries the antisense technology moved from the simple linear oligonucleotides to highly modified polymers with improved membrane penetration, nuclease resistance and stronger base pairing (30-32). The successful design of antisense oligonucleotides depends largely on the ability to identify a primary hybridization site in RNA that is not obstructed by the high order structures (33). This is particularly true for tRNAs that are highly structured since in addition to secondary structure the final L-shaped conformation is held together by tertiary interactions between D- and T-arms and additionally strengthened by Mg²⁺ ions and base-modifications (34).

Based on these considerations we set out to design oligonucleotides targeting isoacceptors from six-fold degenerate codon families that were most suited for reassignment. As the first candidate we chose the tRNA^{Ser}GCU-isoacceptor as its depletion would allow reassignment of AGC(U) codons to uAA while leaving UCN codon box for serine encoding. The antisense oligonucleotides were designed to target the regions spanning from D-stem down to anticodon-loop or from the anticodon-loop to the variable loop (FIG. 1A). The oligonucleotides include DNA-(M1, M6), RNA-(M2, M7), 2′-O-methylated (2′ OMe) nucleotides (M5, M8) as well as mixed DNA/2′OMe (M3) and RNA/2′OMe nucleotides (M4). A Cy3 fluorophore was attached to their 5′-end in order to enable microscale thermophoresis (MST)-based interaction analysis of these polymers with the synthetic tRNA^{Ser}GCU (FIG. 1B).

The M2 (RNA), M4 (partially methylated RNA) and M5 (fully methylated RNA) oligonucleotides targeting the anticodon/variable loop region of tRNA^{Ser}GCU displayed similar K_{d} values of ˜5 nM, while the K_{d} values of the RNA and 2′ OMe-modified oligonucleotides M7 and M8 targeting the D-arm/anticodon region of tRNA were ˜84 and 50 nM, respectively. These results indicate the tRNAs are accessible using antisense oligos with varied efficiency when targeting different regions. The lower efficiency of M7 and M8 is expected as they target an entire D-stem and most likely adopt either hairpin or dimer structure themselves, thus increasing the activation barrier for complex formation with the target. The methylated antisense RNAs demonstrate similar or enhanced affinity towards tRNA^{Ser}GCU compared to their unmodified counterparts. 2′OMe modification may confer enhanced entropic stabilization to the base pairs without sacrificing the base-stacking to acquired structural rigidity (37). Unmodified (M1 and M6) and partially modified (M3) DNA oligonucleotides showed no detectable binding to tRNA^{Ser}GCU at room temperature. This is consistent with the hybrid base-pair stability increasing in the order: DNA/RNA<RNA/RNA (35, 36). The hybridization of unmodified DNA oligonucleotides with their target tRNA may be promoted by use of excess of DNA oligos, heat denaturation and annealing resulting in a stable complex. With further sequence optimisation, or by screening for nucleotide modifications that enhance binding (e.g., Locked Nucleic Acid (LNA), phosphorodiamidate morpholino oligomers (PMOs), phosphorothioate derivatives), it is likely that DNA oligonucleotides that bind to tRNAs could be developed.

In order to confirm the observed interactions using a more direct method we employed a gel-shift assay. As can be seen in FIG. 1C the RNA-(M2, 4, 5, 7, 8) but not DNA-based oligonucleotides (M1, 3, 6) formed stable complex with tRNA. The hybridization of unmodified DNA oligonucleotides with their target tRNA may be promoted by use of excess of DNA oligos, heat denaturation and annealing resulting in a stable complex. In agreement with the determined affinities, oligonucleotides targeting the anticodon/variable loop region (M2, 4, 5) were more efficient binders, with complex formation saturating after incubation for 15 min at 37° C. The oligonucleotides targeting D-arm/anticodon region (M7, 8) displayed weaker interactions and could not be fully complexed even when using 5-fold excess of tRNA^{Ser}GCU.

To confirm the specificity of the observed interactions, we tested the ability of M2 and M5 to recognize the tRNA^{Ser}GGA isoacceptor which is the closest homologue of tRNA^{Ser}GCU showing 46% identity within the targeted anticodon/variable loop region. We observed no complex formation using the gel shift assay (FIG. 1C). In contrast, the D-arm/anticodon region-targeting M8 oligonucleotide displayed some affinity towards tRNA′GGA that shares ˜77% of homology with the tRNA^{Ser}GCU within this region. Neither of these oligonucleotides formed complex with tRNA^{Arg}CCU. These results show that appropriately designed oligonucleotides targeting the less conserved region could selectively form complexes with the target tRNA molecules.

As the fluorescent dye used for oligonucleotide labeling could potentially influence the 5′-end base pairing affecting the affinity measurement (38, 39), we repeated the MST experiments after relocating Cy3 fluorophore to 3′-terminus of tRNA^{Ser}GCU. We monitored the fluorescence change of labelled tRNA while adding increasing concentrations of the unlabeled version of M5 oligonucleotide termed M5-1. The fit of the data resulted in a K_{d} value of 0.365 nM, which is 16 times lower than measured between Cy3-labeled oligonucleotide (M5) and unlabeled tRNA (FIGS. 1B and 1D) indicating the negative impact of the label on complex formation.

### Example 2

Design of the Fluorescent Reporter System for Monitoring of Oligonucleotide-Mediated tRNA Depletion in E. coli Cell-Free Expression System

In the next step we wanted to evaluate the ability of the developed antisense oligonucleotides to inactivate native tRNA^{Ser}GCU in the E. coli in vitro translation system. To this end we designed DNA templates coding for eGFP with biased selection for serine codons. We introduced two AGC-codons decoded by tRNA^{Ser}GCU at position 21 and 22 while encoding remaining serines with one AGT and ten TCC codons (FIG. 2A and Table 2). We expected that translational pausing at the consecutive AGC would lead to the decrease in eGFP expression and serve as a measure of tRNA^{Ser}GCU concentration in the system (22). To confirm that fluorescence reduction is a direct consequence of tRNA^{Ser}GCU sequestration, an efficient AGC-codon suppressor based on tRNA^{Gly}UCC was constructed by replacing its anticodon with GCU (unpublished). Due to its sequence divergence from the tRNA^{Ser}GCU this tRNA wouldn't form a sequence-specific complex with the oligonucleotide and was expected to rescue the eGFP expression if its reduction was caused by ribosome pausing at AGC codons.

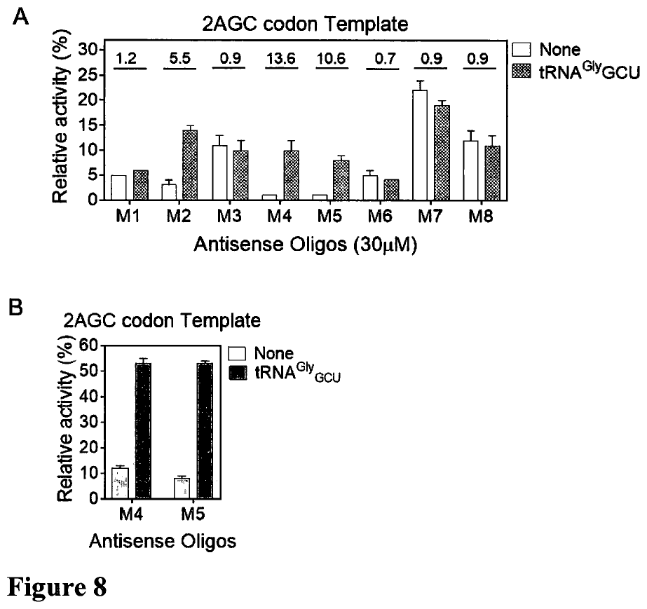

To measure the extent of tRNA depletion using the developed reporter we incubated the lysate with the oligonucleotides at 37° C. for 5 minutes after which the translation reaction was assembled and fluorescence was monitored for 3 hours (FIG. 2B and FIG. 8). We found that the oligonucleotide-mediated fluorescence reduction could be reversed by the addition of synthetic tRNA^{Gly}GCU and the extent of recovery was roughly proportionate to the affinities of the oligonucleotide: tRNA^{Ser}GCU complexes (FIG. 1B and FIG. 8).

Given the importance of methylation for endowing oligonucleotides with high affinity for tRNA, we decided to test the effect of the modification level on the efficiency of translation inhibition and recovery. We designed two partially modified variants of M5-1 based on the alignment between tRNA^{Ser}GCU and the rest of Ser isoacceptors. In these variants termed M5-2 and M5-3 the methylated nucleotides remained at the tRNA^{Ser}GCU-specific positions while the respective 6 (25%) or 13 (>50%) unmodified residues were inserted at the least degenerate positions in an attempt to reduce potential cross-reactivity towards the other tRNA homologs (Table 1). As can be seen in the FIG. 2B we observed a gradual increase in IC₉₀ with the increase in the number of unmodified residues being below 2 μM for M5-1 and M5-2 and >8 μM for M5-3. The recovery of the translational activity with synthetic tRNA^{Gly}GCU suppressor for the first two oligonucleotides reached more than 80% of the untreated reaction (FIG. 2B). The site-selective modifications did not increase the inhibition effect and the fully methylated M5-1 oligonucleotide is so far the most effective tRNA binder designed.

In order to establish whether the developed approach is generally applicable and not confined to tRNA^{Ser}GCU, we selected tRNA^{Arg}CCU which decodes the AGG codon of the mixed codon family Although the AGG-codon is also decoded with lower efficiency by the native tRNA^{Arg}UCU we conjectured that its low abundance in E. coli would allow detection of tRNA^{Arg}CCU inactivation in the translation reaction. Following the considerations described above we designed four 2′OMe antisense oligonucleotides R1-R4 targeting different regions of tRNA^{Arg}CCU from 5′ to 3′ (FIG. 2C). R1 and R3 target two loops of tRNAs while R4 targets the 3′-end. R2 is slightly shifted to the 5′ of the tRNA compared to R3. As shown in the FIG. 2D, R2 inhibited translation of the eGFP template containing a single AGG codon to less than 10% of the original translation levels. The translational inhibition could be reverted by the addition of synthetic tRNA^{Arg}CCU resulting in eGFP production reaching 81% of the untreated lysate. These experiments demonstrate that the methylated oligonucleotide-mediated inactivation of tRNAs is a generally applicable approach.

### Example 3

### Identifying the Essential Functional Elements in the Sequence of M5-1 Oligonucleotide

As the M5-1 antisense oligonucleotide performed very well in in vitro translation reactions we set out to understand its mechanism of action and identify the key structural elements responsible for its activity. M5-1 spans two structurally separate entities of the tRNA that include the anticodon and the variable arms. There is additional complexity introduced by the N₆-threonylcarbamoyl modification of native tRNA^{Ser}GCU on the A37 of the anticodon loop which is expected to destabilize the duplex in this region (42).

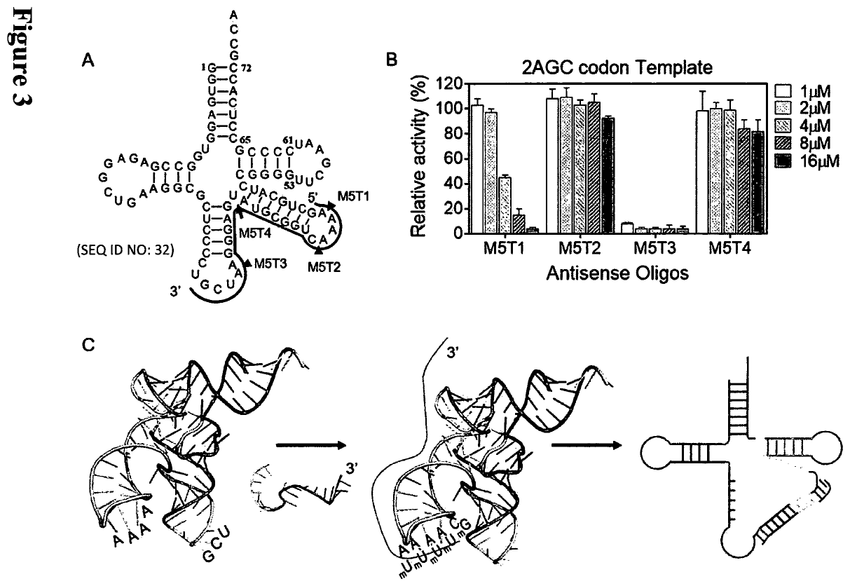

To dissect the structure/function relationship of M5-1 we produced a range of its truncated variants (FIG. 3A). In M5T1 and M5T2 the respectively 5′-dinucleotide or most of the variable loop matching region were deleted. The M5T3 was lacking most of its 3′ anticodon loop complementary region, while M5T4 was entirely devoid of the anticodon arm spanning region. Monitoring the eGFP fluorescence in the translation reaction assay we found that the removal of the most of variable loop complementary fragment (M5T2) or the fragment spanning the anticodon arm (M5T4) led to a dramatic reduction in tRNA targeting efficiency (FIG. 3B). Truncation of only two bases from the 5′ of M5-1 (M5T1) increased the Ic₉₀ from 2 to more than 8 μM (compare FIGS. 2B and 3B). The removal of 3′-terminal anticodon loop hybridizing anchor (M5T3) did not cause any significant change suggesting its functional redundancy.

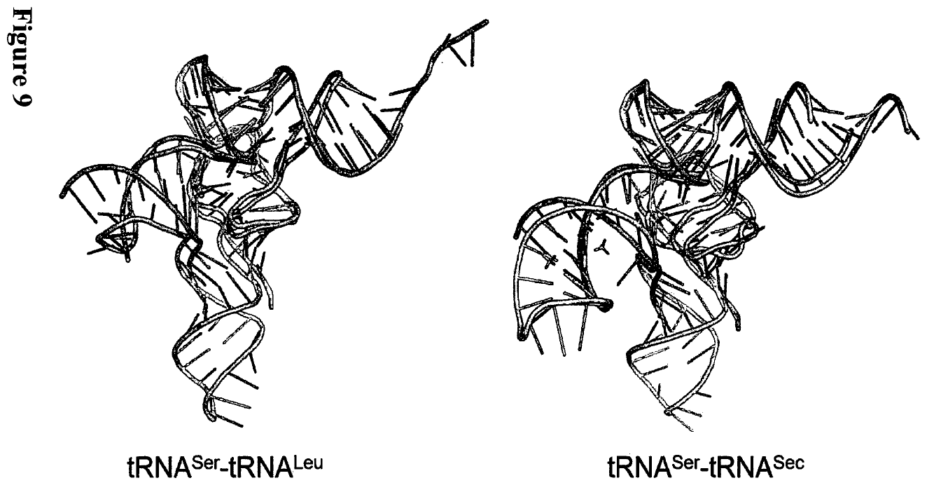

In order to construct a physically meaningful tRNA: oligonucleotide interaction model we examined the crystal structure of tRNA^{Ser}GGA from Thermus thermophiles (PDB: 1Ser) that is the closest homologue of E. coli tRNA^{Ser}GCU (43). The variable loop part is invisible in the T. thermophiles tRNA^{Ser}GGA structure indicating its dynamic nature. In the related structure of tRNA^{Sec} (PDB: 3W3S) (44) the variable tetraloop adopts a classical U-turn with three adenosine bases stacked and exposed to the solvent (FIG. 9). Based on the comparative analysis of these related structures, we conjectured the adenosines of the variable loop of E. coli tRNA^{Ser}GCU are also stacked providing a pre-formed anchoring point for docking of the antisense oligonucleotide.

We proposed that hybridization of the antisense oligonucleotide and tRNA under physiological conditions is hindered by the high activation barrier. The free energy trajectory to the final complex is composed of a number of local equilibriums corresponding to the initial nucleation steps at the accessible tRNA sequence, anchoring and the propagation of the duplex into the stem (FIG. 3C). Our data stresses the importance of the initial anchoring event, where deletion of only two nucleotides from the 5′-terminus significantly reduced the inhibition efficiency while further truncation of the successive three adenosine residues completely abrogated the oligonucleotide's activity. Therefore, four consecutive adenosines are likely to represent a platform for initial anchoring of the oligonucleotide on tRNA similar to that observed in group I introns (45). 2′OMe-residues form base-pairs of higher stability than those of the unmodified nucleotides (46). This is of particular importance for the duplex propagation through the stem by shifting the equilibrium towards hetero-duplex. The truncated oligonucleotide restricted to the variable arm was unable to inhibit tRNA functionality and additional targeting of 3′-part of the anticodon stem was necessary for the activity. This may be a consequence of insufficient destabilization of the overall tRNA tertiary structure that is required for efficient hetero-duplex propagation.

In summary, these results demonstrate that modified antisense oligonucleotides can potently and selectively deactivate selected native E. coli tRNAs directly in the in vitro translation system. The approach is sufficiently simple and scalable to be widely applicable.

### Example 4

Using Oligonucleotide-Mediated tRNA Inactivation for Sense Codon Reassignment In Vitro

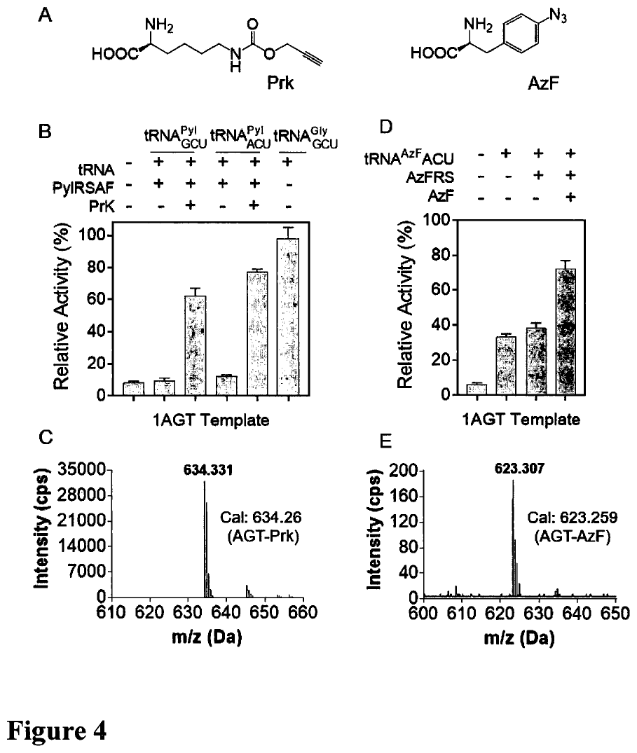

The ability of the antisense oligonucleotides to selectively inactivate the endogenous tRNAs in the E. coli lysate prompted us test the suitability of this approach for incorporation of uAA into polypeptides. To test this, we redesigned the eGFP ORF to include a unique AGT at position 21 (Table 2) and attempted their reassignment to n-propargyl-L-lysine and p-azido-L-phenylalanine by modifying orthogonal tRNA_{CUA}/aaRS pairs established for these uAAs (FIG. 4A) (5).

Reassignment of AGT Codon (Ser) to n-Propargyl-L-Lysine (PrK).

To reassign AGT codon to PrK we used the pyrrolysine (Pyl) system (5) consisting of tRNA^{Pyl} (Table 2), pyrrolysyl-tRNA synthetase variant (PylRSAF) and Prk. As seen in FIG. 4B, M5-1 treated lysate displayed 8% of residual translation efficiency when programed with the template harboring a unique AGT codon thereby indicating an efficient inactivation of native tRNA^{Ser}GCU isoacceptor. Supplementation of the reaction with both PylRSAF and tRNA^{Pyl} did not result in the synthesis of the eGFP reporter indicating their orthogonality to E. coli translational system. Supplementation of such system with PrK restored translation to 80% indicating that ˜90% of protein was modified by Prk. The resulting eGFP protein was purified, trypsin digested and analyzed by LC-MS/MS confirming that Prk efficiently replaced serine at AGT codon (FIG. 4C) as no Ser-containing peptides were detected.

Reassignment of AGT Codon (Ser) to p-Azido-L-Phenylalanine (AzF).

Next we attempted to reassign AGT codon to AzF using tRNA^{Tyr} derivative (5) with grafted ACU anticodon (tRNA^{AzF}ACU) (Table 2) and AzF-tRNA synthetase (AzFRS) derived from tyrosyl-tRNA synthetase of Methanocaldococcus jannaschii (Mj) (47) (FIG. 4D). When M5-1 treated lysate was supplemented with tRNA^{AzF}ACU approximately 35% of eGFP translation was recovered indicating its low orthogonality to E. coli system. Further addition of AzF and AzFRS increased the eGFP expression level to ˜70% indicating occurrence of sense codon suppression via incorporation of AzF at AGT. This was confirmed by the subsequent LC-MS/MS analysis of purified eGFP (FIG. 4E). We conclude that the developed approach can be used to incorporate Prk and AzF into peptides and proteins providing bioorthogonal handles for further chemical and biochemical manipulation with the resulting polypeptides.

### Example 5

Applying Oligonucleotide-Mediated tRNA Inactivation to Eukaryotic Cell-Free Translation Systems

Physical targeting of tRNAs with antisense oligonucleotides in the in vitro translation reaction is expected to be generally applicable due to the conservative nature of the tRNA structure. To test this experimentally we made use of Leishmania tarentolae extract (LTE)-based cell-free translation system developed in our laboratory (27). This system was shown to perform on par with human HeLa system in its ability to produce multi-domain human proteins in folded states (48). The ability to incorporate uAAs into such proteins would be very advantageous for their analysis or endowment with enhanced or novel activities.

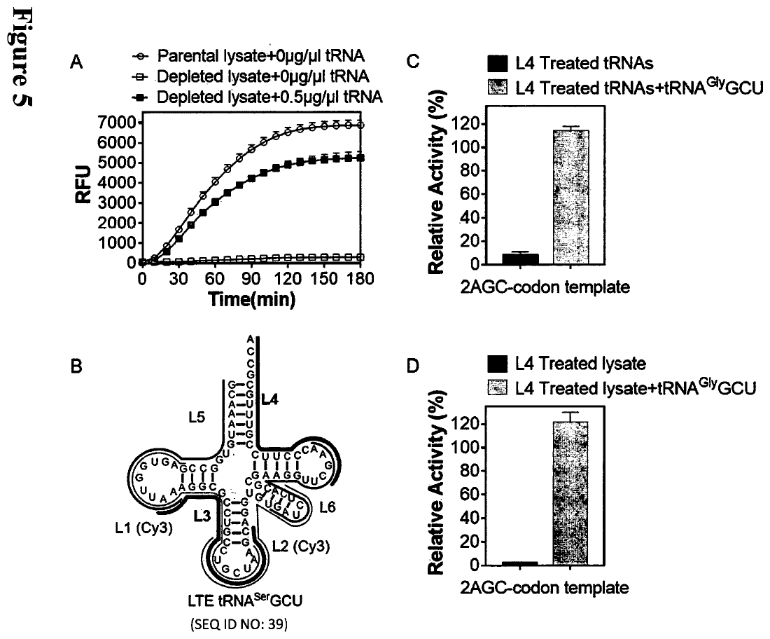

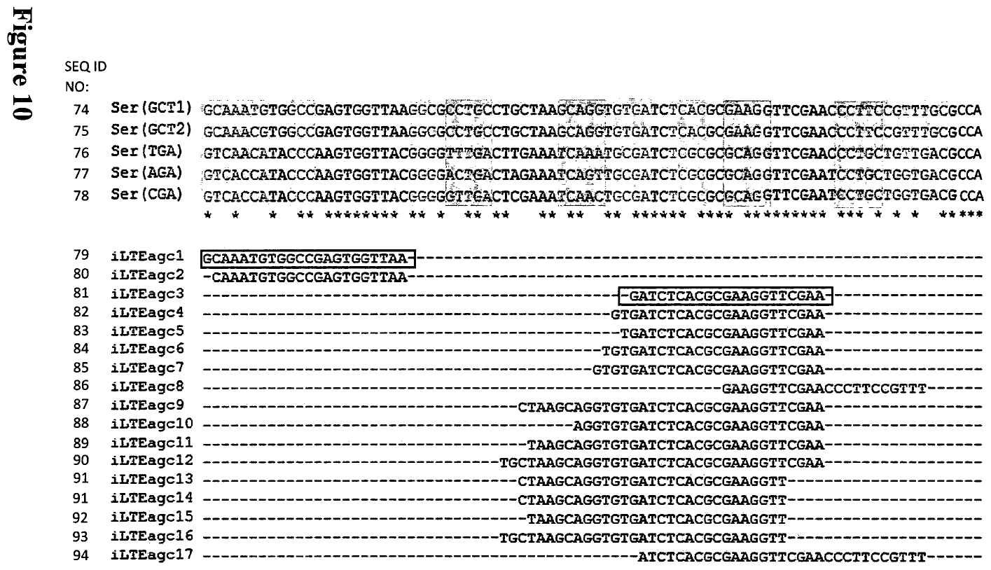

As LTE is much less characterized and more prone to inactivation at prolonged incubations than the E. coli cell-free system we initially sought to devise a “hybrid procedure” that would allow us to manipulate tRNAs without affecting the rest of the translational machinery. Given this, we prepared a tRNA-depleted LTE by using an ethanolamine matrix which non-specifically binds all tRNAs at certain ionic strength (22). As expected, translational activity of such lysate was tRNA-dependent (FIG. 5A). We then manually designed L1 and L2 2′OMe oligonucleotides that target D-loop/anticodon and anticodon/variable loop regions of L. tarentolae tRNA^{Ser}GCU. L3 oligo targeted the 3′- of the D-loop containing several consecutive adenosines while L4 was complementary to the 3′-part of the tRNA (FIG. 5B). Oligonucleotides L5 and L6, were designed using an online RNA design tool, OligoWalk, which utilizes hybridization thermodynamics to predict the best interfering or antisense binders against the target RNA (49) (FIG. 10). We synthesized these oligonucleotides in fully-methylated forms with L1 and 2 also carrying Cy3 fluorophore at the 5′-end (FIG. 5B). The oligonucleotides were tested for their ability to inactivate L. tarentolae tRNA^{Ser}GCU in the context of the unfractionated native tRNAs. Upon incubation at 37° C. for 30 minutes the oligo-tRNA mixtures and the tRNA-depleted LTE were used to reconstitute the in vitro translation system programed with eGFP template carrying two consecutive AGC codons. L4 was the most effective at sequestering tRNA^{Ser}GCU (FIG. 5C) as judged by the reduction in the fluorescence yield to <10% and its recovery upon addition of the chimeric L. tarentolae tRNA^{Gly}CCC with grafted GCU anticodon (tRNA^{Gly}GCU) (FIG. 5C).

We subsequently added the L4 oligonucleotides directly to the LTE lysate and observed significant translational inhibition (FIG. 5D). This inhibition could also be rescued by L. tarentolae tRNA^{Gly}GCU confirming that it was due to selective inactivation of the native tRNA^{Ser}GCU (FIG. 5D). These experiments demonstrate that once hybridized the developed oligonucleotides can efficiently block translation at the chosen codon validating the general applicability of the 2′OMe-oligonucleotide-mediated tRNA sequestration approach.

### Example 6

tRNA Disruption Using RNA Sequences Produced In Vitro by Transcription with T7 Polymerase

As described above, we demonstrated that fully methylated oligonucleotides targeting the tRNA region spanning from the anticodon stem-loop to the variable loop efficiently sequestered E. coli tRNA^{Ser}GCU. These oligonucleotides are commercially synthesized at the cost of ˜$10/nt depending on the scale. Therefore, the cost of synthesising oligonucleotides of a length of 20-25 nt will be ˜$200-250. In contrast, DNA-oligonucleotides are more than 10 times cheaper than the modified or standard RNA oligos. Thus, we wondered whether RNA oligonucleotides synthesised in vitro by T7 RNA polymerase could achieve a similar effect. For these purposes, we designed RNA oligos targeting distinct regions of tRNAs. The DNA templates for these RNA oligonucleotides were provided with T7 promoter coding sequence. Two DNA oligonucleotides of reverse complementary sequences were synthesized commercially and annealed at room temperature after heating up at 95° C. for 5 min. These annealed double-stranded DNAs were used as the templates for T7 transcription.

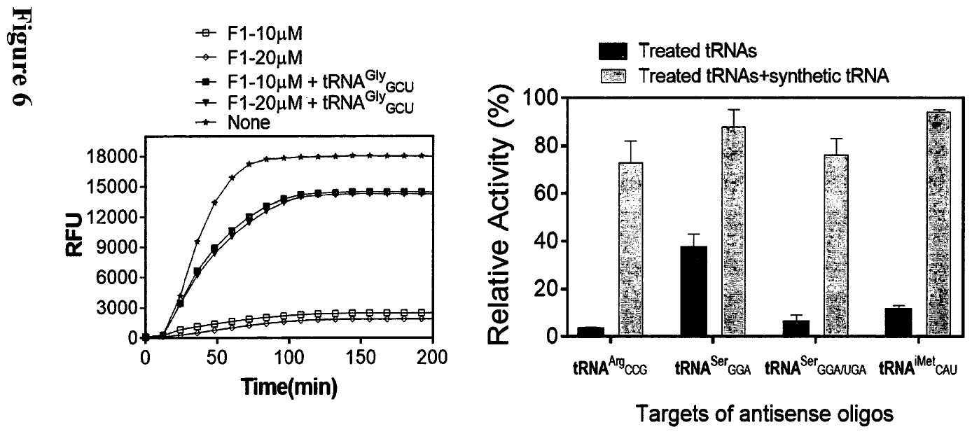

We identified that the T7 transcript termed F1 targeting the tRNA region from the D-arm to the variable loop (N8-47k) could effectively sequester tRNA^{Ser}GCU in the E. coli cell lysate. As shown in FIG. 6A, 10 μM F1 inhibited translation of a 2AGC-codon template to less than 10% of the original translation levels. The translational inhibition could be reverted by the addition of synthetic tRNA^{Gly}GCU resulting in eGFP production reaching to >80% of the level of untreated lysate. Thus, F1 could also be used for tRNA inhibition for sense codon reassignment.

Initial screening of a single T7 transcripts for the other tRNAs, E. coli tRNA^{Ser}GGA, E. coli tRNA^{Ser}UGA, E. coli tRNA^{Arg}CCU, E. coli tRNA^{Arg}CCG, and E. coli tRNA^{iMet}CAU, were partially successful. These RNA transcripts could largely inhibit their respective target tRNA in the context of total native tRNA mixture but to a lesser extent in the cell lysate. As shown in FIG. 6B, cell-free translation reaction reconstituted of all-tRNA-depleted lysate and antisense oligonucleotide-treated total native tRNA mix showed inhibition of translation at codons CGG, UCC, AUG (start codon) at 3%, 37%, and 11%, respectively. Total native tRNA mixture treated with the pair of antisense oligonucleotides targeting both tRNA′GGA and tRNA^{Ser}UGA showed inhibition of translation of UCC codon up to 6% confirming the tRNA^{Ser}UGA participation in reading by both the two tRNA isoacceptors. Translational recovery using the corresponding synthetic tRNAs was achieved to more than 70%. Therefore, we confirmed the utility of T7 transcription as a method to synthesise the antisense oligonucleotides. Such a method is more economical compared to synthesising the methylated oligonucleotides, and is suitable for synthesising RNA libraries.

### Discussion

In this study we present a new approach for sense codon reassignment. The approach is based on the idea that individual tRNA isoacceptors can be selectively inactivated by the antisense oligonucleotides, e.g., in the context of in vitro translation system and replaced with their synthetic orthogonal analogs carrying unnatural amino acids.

We demonstrate the generation of oligonucleotide M5-1 that binds E. coli tRNA^{Ser}GCU with subnanomolar affinity. The oligonucleotide shows no detectable affinity to its closest homologue tRNA′GGA. These parameters enable selective tRNA inactivation in the translation system. We demonstrate the oligonucleotide deployment directly in the S30 E. coli lysate with no observable off-target effects and confirm the general applicability of this approach by creating inhibitory oligonucleotides against E. coli tRNA^{Arg}CCU. The best oligonucleotides target the sequences from the anticodon stem to the beginning of T-loop of tRNA^{Arg}CCU.

The truncation analysis allowed us to propose a mechanism for oligonucleotide-mediated tRNA^{Ser}GCU sequestration. According to our model the single-stranded oligonucleotide binds the solvent-facing stack of nucleotide bases of the tRNA loops. Base-paring propagation from the loop into the double stranded and higher order structure-constrained parts is necessary for tRNA unfolding. The base modifications contribute to the stability of the resulting hetero-duplex and prevent tRNA's return to its native conformation. Importantly, the successful oligonucleotide designs were able to suppress over 90% protein production that can be restored to nearly original levels with orthogonal synthetic suppressor tRNAs.

In order to achieve robust uAA incorporation, the heterogeneous tRNA with corresponding anticodon should be orthogonal to the endogenous system as well as efficiently charged with uAAs by the orthogonal aaRS. We demonstrated that orthogonality of tRNA^{Pyl} with ACU anticodon was as high as in the case with amber anti-codon. The n-propargyl-L-lysine charged tRNA^{Pyl} ACU could support AGT codon suppression in the oligonucleotide treated lysate reaching ˜80% suppression efficiency of the wild-type eGFP production in the untreated lysate. The resulting protein was homogeneously modified with Prk, as judged from the LC-MS/MS results—indicating high efficiency of sense codon reassignment. Further experiments demonstrated that ACU anticodon transplantation onto the orthogonal amber suppressor tRNA from M. jannaschii led to unspecific recognition by the endogenous E. coli aaRS. However, supplementation of p-azido-L-phenylalanine to the reaction containing both tRNA^{AzF}ACU and AzFRS enabled its incorporation at the position of AGT codon. This indicated that AzFRS charged o-tRNA was able to outcompete the aminoacylated tRNA pool produced by the endogenous aaRSs. This is not surprising as the presence of high concentrations of uAA and o-aaRS is expected to shift the balance toward the uAA incorporation by outcompeting the endogenous aaRSs mischarging o-tRNA with natural amino acids (50, 51).

We subsequently applied the developed approach to inactivate L. tarentolae tRNA^{Ser}GCU both in the total tRNA mixture or L. tarentolae cell extract. The antisense oligonucleotides were effective in inactivating the selected tRNAs in both cases thereby liberating AGC/AGT codons. This demonstrates that the developed approach is not restricted to a particular cell-free system and is generally applicable.

The best antisense oligonucleotides identified for E. coli tRNA^{Ser}GCU, E. coli tRNA^{Arg}CCU, L. tarentolae tRNA^{Ser}GCU are not targeting the same sequence motifs of their corresponding tRNA targets. The most accessible single-stranded regions of these tRNAs occur in the variable loop, anticodon loop or 3′-CCA end. Rational design of oligonucleotides needs to be combined with the functional screening to identify the best performing oligonucleotides. It is worth noting that the algorithm-based oligonucleotide design implemented in the Oligo-Walk failed to produce efficient tRNA binders. Future systematic analysis of 12 to 30 nts RNA oligonucleotide arrays spanning the entire tRNAs appear to be a promising strategy for identifying the most effective oligonucleotides and establishing their design criteria. Besides 2′-O-methylation, exploration of the additional oligonucleotide chemistries such as Locked Nucleic Acid (LNA), phosphorodiamidate morpholino oligomers (PMOs) as well as phosphorothioate derivatives (32, 52, 53) with a goal of increasing the hetero-duplex stability and reducing their nucleolytic degradation may provide even more efficient inhibitory oligonucleotides to other tRNA species.

In conclusion, the approach reported here stands out in its simplicity as it only requires a short incubation of the cell-free lysate with oligonucleotides before assembly of the translation reaction. The approach provides a new avenue for sense codon reassignment and uAA incorporation into recombinant polypeptides. Unlike previously reported approaches, this method is chromatography independent and fully scalable, potentially allowing its industrial utilization. This opens up a route for creation of peptides and proteins with novel properties, such as drug-antibody conjugates, bioactive peptides with improved bioavailability, synthetic vaccines as well as novel enzymes with enhanced or novel catalytic activities.

### REFERENCES

1. Liu, C. C. and Schultz, P. G. (2010) Adding New Chemistries to the Genetic Code. Annu Rev Biochem, 79, 413-444.
2. O'Donoghue, P., Ling, J., Wang, Y. S. and Soll, D. (2013) Upgrading protein synthesis for synthetic biology. Nat Chem Biol, 9, 594-598.
3. Dumas, A., Lercher, L., Spicer, C. D. and Davis, B. G. (2015) Designing logical codon reassignment—Expanding the chemistry in biology. Chem. Sci., 6, 50-69.
4. Mukai, T., Yamaguchi, A., Ohtake, K., Takahashi, M., Hayashi, A., Iraha, F., Kira, S., Yanagisawa, T., Yokoyama, S., Hoshi, H. et al. (2015) Reassignment of a rare sense codon to a non-canonical amino acid in Escherichia coli. Nucleic Acids Res, 43, 8111-8122.
5. Cui, Z., Mureev, S., Polinkovsky, M. E., Tnimov, Z., Guo, Z., Durek, T., Jones, A. and Alexandrov, K. (2017) Combining Sense and Nonsense Codon Reassignment for Site-Selective Protein Modification with Unnatural Amino Acids. ACS Synth Biol, 6, 535-544.
6. Iwane, Y., Hitomi, A., Murakami, H., Katoh, T., Goto, Y. and Suga, H. (2016) Expanding the amino acid repertoire of ribosomal polypeptide synthesis via the artificial division of codon boxes. Nat Chem, 8, 317-325.
7. Zeng, Y., Wang, W. and Liu, W. R. (2014) Towards reassigning the rare AGG codon in Escherichia coli. Chembiochem: a European journal of chemical biology, 15, 1750-1754.
8. Lee, B. S., Shin, S., Jeon, J. Y., Jang, K. S., Lee, B. Y., Choi, S. and Yoo, T. H. (2015) Incorporation of Unnatural Amino Acids in Response to the AGG Codon. ACS Chem Biol, 10, 1648-1653.
9. Ostrov, N., Landon, M., Guell, M., Kuznetsov, G., Teramoto, J., Cervantes, N., Zhou, M., Singh, K., Napolitano, M. G., Moosburner, M. et al. (2016) Design, synthesis, and testing toward a 57-codon genome. Science, 353, 819-822.
10. Phizicky, E. M. and Hopper, A. K. (2010) tRNA biology charges to the front. Gene Dev, 24, 1832-1860.
11. Neumann, H., Wang, K., Davis, L., Garcia-Alai, M. and Chin, J. W. (2010) Encoding multiple unnatural amino acids via evolution of a quadruplet-decoding ribosome. Nature, 464, 441-444.
12. Malyshev, D. A., Dhami, K., Lavergne, T., Chen, T., Dai, N., Foster, J. M., Correa, I. R., Jr. and Romesberg, F. E. (2014) A semi-synthetic organism with an expanded genetic alphabet. Nature, 509, 385-388.
13. Malyshev, D. A. and Romesberg, F. E. (2015) The expanded genetic alphabet. Angew Chem Int Ed Engl, 54, 11930-11944.
14. Schwarz, D., Junge, F., Durst, F., Frolich, N., Schneider, B., Reckel, S., Sobhanifar, S., Dotsch, V. and Bernhard, F. (2007) Preparative scale expression of membrane proteins in Escherichia coli-based continuous exchange cell-free systems. Nat Protoc, 2, 2945-2957.
15. Quast, R. B., Mrusek, D., Hoffmeister, C., Sonnabend, A. and Kubick, S. (2015) Cotranslational incorporation of non-standard amino acids using cell-free protein synthesis. FEBS letters, 589, 1703-1712.
16. Loscha, K. V., Herlt, A. J., Qi, R., Huber, T., Ozawa, K. and Otting, G. (2012) Multiple-site labeling of proteins with unnatural amino acids. Angew Chem Int Ed Engl, 51, 2243-2246.
17. Carlson, E. D., Gan, R., Hodgman, C. E. and Jewett, M. C. (2012) Cell-free protein synthesis: applications come of age. Biotechnol Adv, 30, 1185-1194.
18. Hong, S. H., Kwon, Y. C. and Jewett, M. C. (2014) Non-standard amino acid incorporation into proteins using Escherichia coli cell-free protein synthesis. Front Chem, 2, 34.
19. Kanda, T., Takai, K., Yokoyama, S. and Takaku, H. (2000) An easy cell-free protein synthesis system dependent on the addition of crude Escherichia coli tRNA. J Biochem, 127, 37-41.
20. Salehi, A. S. M., Smith, M. T., Schinn, S. M., Hunt, J. M., Muhlestein, C., Diray-Arce, J., Nielsen, B. L. and Bundy, B. C. (2017) Efficient tRNA degradation and quantification in Escherichia coli cell extract using RNase-coated magnetic beads: A key step toward codon emancipation. Biotechnol Prog, 33, 1401-1407.
21. Frankel, A. and Roberts, R. W. (2003) In vitro selection for sense codon suppression. RNA, 9, 780-786.
22. Cui, Z., Stein, V., Tnimov, Z., Mureev, S. and Alexandrov, K. (2015) Semisynthetic tRNA complement mediates in vitro protein synthesis. J Am Chem Soc, 137, 4404-4413.
23. Ahn, J. H., Hwang, M. Y., Oh, I. S., Park, K. M., Hahn, G. H., Cho, C. Y. and Kim, D. M. (2006) Preparation method for Escherichia coli S30 extracts completely dependent upon tRNA addition to catalyze cell-free protein synthesis. Biotechnol Bioproc E, 11, 420-424.
24. Kanda, T., Takai, K., Hohsaka, T., Sisido, M. and Takaku, H. (2000) Sense codon-dependent introduction of unnatural amino acids into multiple sites of a protein. Biochem Biophys Res Commun, 270, 1136-1139.
25. Thisted, T., Sorensen, N. S., Wagner, E. G. and Gerdes, K. (1994) Mechanism of post-segregational killing: Sok antisense RNA interacts with Hok mRNA via its 5′-end single-stranded leader and competes with the 3′-end of Hok mRNA for binding to the mok translational initiation region. Embo J, 13, 1960-1968.
26. Kubala, M. H., Kovtun, O., Alexandrov, K. and Collins, B. M. (2010) Structural and thermodynamic analysis of the GFP:GFP-nanobody complex. Protein Sci, 19, 2389-2401.
27. Mureev, S., Kovtun, O., Nguyen, U. T. and Alexandrov, K. (2009) Species-independent translational leaders facilitate cell-free expression. Nat Biotechnol, 27, 747-752.
28. Zubay, G. (1962) Isolation and Fractionation of Soluble Ribonucleic Acid. J Mol Biol, 4, 347-&.
29. Bennett, C. F. and Swayze, E. E. (2010) RNA targeting therapeutics: molecular mechanisms of antisense oligonucleotides as a therapeutic platform. Annu Rev Pharmacol Toxicol, 50, 259-293.
30. Rasmussen, L. C., Sperling-Petersen, H. U. and Mortensen, K. K. (2007) Hitting bacteria at the heart of the central dogma: sequence-specific inhibition. Microb Cell Fact, 6, 24.
31. Khorkova, O. and Wahlestedt, C. (2017) Oligonucleotide therapies for disorders of the nervous system. Nat Biotechnol, 35, 249-263.
32. Lundin, K. E., Gissberg, O. and Smith, C. I. (2015) Oligonucleotide Therapies: The Past and the Present. Hum Gene Ther, 26, 475-485.
33. Sczakiel, G. and Far, R. K. (2002) The role of target accessibility for antisense inhibition. Curr Opin Mol Ther, 4, 149-153.
34. Giege, R., Juhling, F., Putz, J., Stadler, P., Sauter, C. and Florentz, C. (2012) Structure of transfer RNAs: similarity and variability. Wiley Interdiscip Rev RNA, 3, 37-61.
35. Lesnik, E. A. and Freier, S. M. (1995) Relative thermodynamic stability of DNA, RNA, and DNA:RNA hybrid duplexes: relationship with base composition and structure. Biochemistry, 34, 10807-10815.
36. Acharya, P., Cheruku, P., Chatterjee, S., Acharya, S. and Chattopadhyaya, J. (2004) Measurement of nucleobase pKa values in model mononucleotides shows RNA-RNA duplexes to be more stable than DNA-DNA duplexes. J Am Chem Soc, 126, 2862-2869.
37. Yildirim, I., Kierzek, E., Kierzek, R. and Schatz, G. C. (2014) Interplay of LNA and 2′-O-methyl RNA in the structure and thermodynamics of RNA hybrid systems: a molecular dynamics study using the revised AMBER force field and comparison with experimental results. J Phys Chem B, 118, 14177-14187.
38. Spiriti, J., Binder, J. K., Levitus, M. and van der Vaart, A. (2011) Cy3-DNA stacking interactions strongly depend on the identity of the terminal basepair. Biophys J, 100, 1049-1057.
39. Milas, P., Gamari, B. D., Parrot, L., Krueger, B. P., Rahmanseresht, S., Moore, J. and Goldner, L. S. (2013) Indocyanine dyes approach free rotation at the 3′ terminus of A-RNA: a comparison with the 5′ terminus and consequences for fluorescence resonance energy transfer. J Phys Chem B, 117, 8649-8658.
40. Pan, A. C., Borhani, D. W., Dror, R. O. and Shaw, D. E. (2013) Molecular determinants of drug-receptor binding kinetics. Drug Discov Today, 18, 667-673.
41. Copeland, R. A., Pompliano, D. L. and Meek, T. D. (2006) Drug-target residence time and its implications for lead optimization. Nat Rev Drug Discov, 5, 730-739.
42. Murphy, F. V. t., Ramakrishnan, V., Malkiewicz, A. and Agris, P. F. (2004) The role of modifications in codon discrimination by tRNA(Lys)UUU. Nat Struct Mol Biol, 11, 1186-1191.
43. Biou, V., Yaremchuk, A., Tukalo, M. and Cusack, S. (1994) The 2.9 A crystal structure of T. thermophilus seryl-tRNA synthetase complexed with tRNA(Ser). Science, 263, 1404-1410.
44. Itoh, Y., Sekine, S., Suetsugu, S. and Yokoyama, S. (2013) Tertiary structure of bacterial selenocysteine tRNA. Nucleic Acids Res, 41, 6729-6738.
45. Gate, J. H., Gooding, A. R., Podell, E., Zhou, K. H., Golden, B. L., Szewczak, A. A., Kundrot, C. E., Cech, T. R. and Doudna, J. A. (1996) RNA tertiary structure mediation by adenosine platforms. Science, 273, 1696-1699.
46. Majlessi, M., Nelson, N.C. and Becker, M. M. (1998) Advantages of 2′-O-methyl oligoribonucleotide probes for detecting RNA targets. Nucleic Acids Res, 26, 2224-2229.
47. Chin, J. W., Santoro, S. W., Martin, A. B., King, D. S., Wang, L. and Schultz, P. G. (2002) Addition of p-azido-L-phenylalanine to the genetic code of Escherichia coli. J Am Chem Soc, 124, 9026-9027.
48. Gagoski, D., Polinkovsky, M. E., Mureev, S., Kunert, A., Johnston, W., Gambin, Y. and Alexandrov, K. (2016) Performance benchmarking of four cell-free protein expression systems. Biotechnol Bioeng, 113, 292-300.
49. Lu, Z. J. and Mathews, D. H. (2008) OligoWalk: an online siRNA design tool utilizing hybridization thermodynamics. Nucleic Acids Res, 36, W104-108.
50. Sherman, J. M., Rogers, M. J. and Soll, D. (1992) Competition of aminoacyl-tRNA synthetases for tRNA ensures the accuracy of aminoacylation. Nucleic Acids Res, 20, 2847-2852.
51. Wohlgemuth, I., Pohl, C., Mittelstaet, J., Konevega, A. L. and Rodnina, M. V. (2011) Evolutionary optimization of speed and accuracy of decoding on the ribosome. Philos T R Soc B, 366, 2979-2986.
52. Vester, B. and Wengel, J. (2004) LNA (locked nucleic acid): high-affinity targeting of complementary RNA and DNA. Biochemistry, 43, 13233-13241.
53. Eckstein, F. (2014) Phosphorothioates, essential components of therapeutic oligonucleotides. Nucleic Acid Ther, 24, 374-387.

### SEQUENCE LISTING

Oligonucleotide Sequences
(M1)
SEQ ID NO: 1
/5Cy3/dCdTdTdTdTdGdAdCdCdGdCdAdTdAdCdTdCdCdCdTdTdAdGdC
(M2)
SEQ ID NO: 2
/5Cy3/rCrUrUrUrUrGrArCrCrGrCrArUrArCrUrCrCrCrUrUrArGrC
(M3)
SEQ ID NO: 3
/5Cy3/mCmUmUmUdTdGdAdCdCdGdCdAdTdAdCdTdCdCdCdTmUmAmGmC
(M4)
SEQ ID NO: 4
/5Cy3/mCmUmUmUrUrGrArCrCrGrCrArUrArCrUrCrCrCrUmUmAmGmC
(M5)
SEQ ID NO: 5
/5Cy3/mCmUmUmUmUmGmAmCmCmGmCmAmUmAmCmUmCmCmCmUmUmAm
GmC
(M6)
SEQ ID NO: 6
/5Cy3/dAdGdCdAdGdGdGdGdAdGdCdGdCdCdTdTdCdAdGdCdCdTdCdTdCdGdGdCd
CdA
(M7)
SEQ ID NO: 7
/5Cy3/rArGrCrArGrGrGrGrArGrCrGrCrCrUrUrCrArGrCrCrUrCrUrCrGrGrCrCrA
(M8)
SEQ ID NO: 8
/5Cy3/mAmGmCmAmGmGmGmGmAmGmCmGmCmCmUmUmCmAmGmCmCmUm
CmUmCmGmGmCmCmA
(M5-1)
SEQ ID NO: 9
mCmUmUmUmUmGmAmCmCmGmCmAmUmAmCmUmCmCmCmUmUmAmGmC
(M5-2)
SEQ ID NO: 10
mCmUmUmUmUmGmArCrCmGmCrAmUmArCmUmCmCmCrUrUmAmGmC
(M5-3)
SEQ ID NO: 11
mCmUmUmUrUmGmArCrCrGmCrArUrArCmUrCmCrCrUrUmAmGrC
(M5T1)
SEQ ID NO: 12
mUmUmUmGmAmCmCmGmCmAmUmAmCmUmCmCmCmUmUmAmGmC/3AmMO/
(M5T2)
SEQ ID NO: 13
mGmAmCmCmGmCmAmUmAmCmUmCmCmCmUmUmAmGmC/3AmMO/
(M5T3)
SEQ ID NO: 14
mCmUmUmUmUmGmAmCmCmGmCmAmUmAmCmUmCmCmCmU/3AmMO/
(M5T4)
SEQ ID NO: 15
mCmUmUmUmUmGmAmCmCmGmCmAmU/3AmMO/
(MF5)
SEQ ID NO: 16
fCfUfUfUfUfGfAfCfCfGfCfAfUfAfCfUfCfCfCfUfUfAfGfC
(R1)
SEQ ID NO: 17
mAmGmGmAmGmGmGmGmCmUmCmGmUmUmAmUmAmUmCmCmAmUmUmU
(R2)
SEQ ID NO: 18
mCmCmUmGmCmAmAmUmUmAmGmCmCmCmUmUmAmGmGmAmGmG
(R3)
SEQ ID NO: 19
mAmUmCmGmAmAmCmCmUmGmCmAmAmUmUmAmGmCmCmCmUmUmAmGm
G
(R4)
SEQ ID NO: 20
mUmGmGmUmGmUmCmCmCmCmUmGmCmAmGmGmAmAmUmCmGmAmAmC
(L1)
SEQ ID NO: 21
/5Cy3/mGmAmGmAmUmCmAmCmAmCmCmUmGmCmUmUmAmGmCmAmG
(L2)
SEQ ID NO: 22
/5Cy3/mUmUmAmGmCmAmGmGmCmAmGmGmCmGmCmCmUmUmAmAmCmCm
AmCmUmC
(L3)
SEQ ID NO: 23
mGmCmUmUmAmGmCmAmGmGmCmAmGmGmCmGmCmCmUmU
(L4)
SEQ ID NO: 24
mUmGmGmCmGmCmAmAmAmCmGmGmAmAmGmGmGmUmUmCmG
(L5)
SEQ ID NO: 25
mUmUmAmAmCmCmAmCmUmCmGmGmCmCmAmCmAmUmUmUmGmC
(L6)
SEQ ID NO: 26
mUmUmCmGmAmAmCmCmUmUmCmGmCmGmUmGmAmGmAmUmC
(oligo E. coli tRNA^{Ser}GCU-F1)
SEQ ID NO: 27
GCUUUUGACCGCAUACUCCCUUAGCAGGGGAGCGCCUUCAGCCUCUCGGCCA
(oligo E. coli tRNA^{Ser}GGA-F6)
SEQ ID NO: 28
GCUUUCCAGGCGUGCUCCUUCAGCCACUCGGACACGUCACC
(oligo E. coli tRNA^{Ser}UGA-F2)
SEQ ID NO: 29
GCUUUCGGGUCGCCGGUUUUCAAGACCGGUGCCUUCAACCGCUCGGCCA
(oligo E. coli tRNA^{Arg}CCG-F1)
SEQ ID NO: 30
GACCUCUGCCUCCGGAGGGCAGCGCUCUAUCCAGCUGAGCUA
(oligo tRNA^{iMet}CAU-F1)
SEQ ID NO: 31
GACCUUCGGGUUAUGAGCCCGACGAGCUACCAGGCUGCUCCA

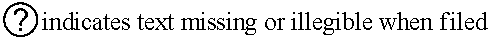

Wherein dN = deoxyribonucleotide; rN = ribonucleotide; mN = 2′-O-methyl ribonucleotide
Wherein /3AmMO/ = 3′ Amino Modifier 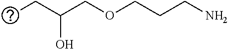
Wherein /5Cy3/ = 5′ Cy3 fluorophore 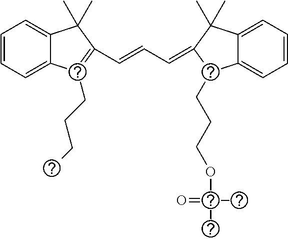
Wherein fN = 2′-fluoro ribonucleotide

tRNA Sequences

SEQ ID NO: 32 (E. coli tRNA^{Ser}GCU)
GGUGAGGUGGCCGAGAGGCUGAAGGCGCUCCCCUGCUAAGGGAGUAUGCGG
UCAAAAGCUGCAUCCGGGGUUCGAAUCCCCGCCUCACCGCCA
SEQ ID NO: 33 (wild type (wt) E. coli tRNA^{Gly}UCC)
GCGGGCAUCGUAUAAUGGCUAUUACCUCAGCCUUCCAAGCUGAUGAUGCGG
GUUCGAUUCCCGCUGCCCGCUCCA
SEQ ID NO: 34 (wt E. coli tRNA^{Ser}GGA)
GGUGAGGUGUCCGAGUGGCUGAAGGAGCACGCCUGGAAAGUGUGUAUACGG
CAACGUAUCGGGGGUUCGAAUCCCCCCCUCACCGCCA
SEQ ID NO: 35 (wt E. coli tRNA^{Ser}UGA)
GGAAGUGUGGCCGAGCGGUUGAAGGCACCGGUCUUGAAAACCGGCGACCCG
AAAGGGUUCCAGAGUUCGAAUCUCUGCGCUUCCGCCA
SEQ ID NO: 36 (E. coli tRNA^{Arg}CCU)
GUCCUCUUAGUUAAAUGGAUAUAACGAGCCCCUCCUAAGGGCUAAUUGCAG
GUUCGAUUCCUGCAGGGGACACCA
SEQ ID NO: 37 (wt E. coli tRNA^{Arg}CCG)
GCGCCCGUAGCUCAGCUGGAUAGAGCGCUGCCCUCCGGAGGCAGAGGUCUC
AGGUUCGAAUCCUGUCGGGCGCGCCA
SEQ ID NO: 38 (wt E. coli tRNA^{iMet}CAU)
CGCGGGGUGGAGCAGCCUGGUAGCUCGUCGGGCUCAUAACCCGAAGGUCGU
CGGUUCAAAUCCGGCCCCCGCAACCA
SEQ ID NO: 39 (wt L. tarentolae (LTE) tRNA^{Ser}GCU)
GCAAAUGUGGCCGAGUGGUUAAGGCGCCUGCCUGCUAAGCAGGUGUGAUCU
CACGCGAAGGUUCGAACCCUUCCGUUUGCG
SEQ ID NO: 40 (wt L. tarentolae (LTE) tRNA^{Ser}GCU 2)
GCAAACGUGGCCGAGUGGUUAAGGCGCCUGCCUGCUAAGCAGGUGUGAUCU
CACGCGAAGGUUCGAACCCUUCCGUUUGCGCCA
SEQ ID NO: 41 (wt L. tarentolae (LTE) tRNA^{Ser}AGA)
GUCACCAUACCCAAGUGGUUACGGGGACUGACUAGAAAUCAGUUGCGAUCU
C GCGC GC AGGUUC GAAUCCUGCUGGUGACGCC A
SEQ ID NO: 42 (wt L. tarentolae (LTE) tRNA^{Ser}CGA)
GUCACCAUACCCAAGUGGUUACGGGGGUUGACUCGAAAUCAACUGCGAUCU
CGCGCGCAGGUUCGAAUCCUGCUGGUGACGCCA
SEQ ID NO: 43 (wt L. tarentolae (LTE) tRNA^{Ser}UGA)
GUCAACAUACCCAAGUGGUUACGGGGUUUGACUUGAAAUCAAAUGCGAUCU
CGCGCGCAGGUUCGAACCCUGCUGUUGACGCCA
SEQ ID NO: 44 (wt LTE tRNA^{Gly}CCC)
GCGCCGCUGGUCUAGUGGCAUGAUGGUACCCUCCCAAGGUAUUGACCCGGG
UUCGAUUCCCGGGCGGCGCACCA
SEQ ID NO: 45 (synthetic E. coli tRNA^{GlyG}CU)
GCGGGCAUCGUAUAAUGGCUAUUACCUCAGCCUGCUAAGCUGAUGAUGCGG
GUUCGAUUCCCGCUGCCCGCUCCA
SEQ ID NO: 46 (synthetic LTE tRNA^{GlyG}GCU)
GCGCCGCUGGUCUAGUGGCAUGAUGGUACCCUGCUAAGGUAUUGACCCGGG
UUCGAUUCCCGGGCGGCGCACCA
SEQ ID NO: 47 (synthetic tRNA^{AzF}ACU)
CCGGCCGUAGUUCAGCAGGGCAGAACGGCGGACUACUAAUCCGCAUGGCAU
GGGUUCAAAUCCCAUCGGCCGGACCA
SEQ ID NO: 48 (synthetic tRNA^{AzF}GCU)
CCGGCCGUAGUUCAGCAGGGCAGAACGGCGGACUGCUAAUCCGCAUGGCAU
GGGUUCAAAUCCCAUCGGCCGGACCA
SEQ ID NO: 49 (synthetic tRNA^{Pyl}ACU)
GGAAACCUGAUCAUGUAGAUCGAACGGACUACUAAUCCGUUCAGCCGGGUU
AGAUUCCCGGGGUUUCCGCCA
SEQ ID NO: 50 (synthetic tRNA^{Pyl}GCU)
GGAAACCUGAUCAUGUAGAUCGAACGGACUGCUAAUCCGUUCAGCCGGGUU
AGAUUCCCGGGGUUUCCGCCA
Wherein NNN = tRNA anticodon triplet

Fluorescent Reporter Sequences for Protein Translation Assay

SEQ ID NO: 51 (T7 promoter)
TAATACGACTCACTATA
SEQ ID NO: 52 (SITS sequence)
TTTATTTTATTTTATTTTATTTTATTTTATTTTATTTTATTTTATTTTATTTTAATT
AATTTTATTTAACCATGACAGTAATGTATAAAGTCTGTAAAGACATTAAACAC
GTAAGTGAAACC
Wherein NNN = start codon.
SEQ ID NO: 53 (SITS protein sequence)
MTVMYKVCKDIKHVSET
SEQ ID NO: 54 (2AGC codon eGFP ORF)
ATGACAGTAATGTATAAAGTCTGTAAAGACATTAAACACGTAagtGAAACCATGGAGA
TCagcagcAAGGGCGAGGAGCTGTTCACCGGGGTGGTGCCCATCCTGGTCGAGCT
GGACGGCGACGTAAACGGCCACAAGTTCtccGTGtccGGCGAGGGCGAGGGCGAT
GCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCtcc
CGCTACCCCGACCACATGAAGCAGCACGACTTCTTCAAGtccGCCATGCCCGAA
GGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTG
AAGGGCATCGACTTCAAGGAGGACGGCAACATCCTGGGGCACAAGCTGGAGT
ACAACTACAACtccCACAACGTCTATATCATGGCCGACAAGCAGAAGAACGGCA
TCAAGGTGAACTTCAAGATCCGCCACAACATCGAGGACGGCtccGTGCAGCTCG
CCGACCACTACCAGCAGAACACCCCCATCGGCGACGGCCCCGTGCTGCTGCCC
GACAACCACTACCTGtccACCCAGtccGCCCTGtccAAAGACCCCAACGAGAAGCG
CGATCACATGGTCCTGCTGGAGTTCGTGACCGCCGCCGGGATCACTCTCGGCA
TGGACGAGCTATACAAGGAGCAGAAGCTGATCtccGAGGAGGATCTGTAA
Wherein residues in italics are the 3′-SITS sequence. This is followed by the eGFP coding
sequence. Ser codons in the ORF are shown in lower case: 2 AGC; 1 AGT; 10 TCC.
SEQ ID NO: 55 (2AGC DNA template sequence)
GACGTCTAATACGACTCACTATAGGGACATCTTAAGTTTATTTTATTTTATTTTA
TTTTATTTTATTTTATTTTATTTTATTTTATTTTAATTAATTTTATTTAACCATGA
CAGTAATGTATAAAGTCTGTAAAGACATTAAACACGTAagtGAAACCATGGAGA
TCagcagcAAGGGCGAGGAGCTGTTCACCGGGGTGGTGCCCATCCTGGTCGAGCT
GGACGGCGACGTAAACGGCCACAAGTTCtccGTGtccGGCGAGGGCGAGGGCGAT
GCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCtcc
CGCTACCCCGACCACATGAAGCAGCACGACTTCTTCAAGtccGCCATGCCCGAA
GGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTG
AAGGGCATCGACTTCAAGGAGGACGGCAACATCCTGGGGCACAAGCTGGAGT
ACAACTACAACtccCACAACGTCTATATCATGGCCGACAAGCAGAAGAACGGCA
TCAAGGTGAACTTCAAGATCCGCCACAACATCGAGGACGGCtccGTGCAGCTCG
CCGACCACTACCAGCAGAACACCCCCATCGGCGACGGCCCCGTGCTGCTGCCC
GACAACCACTACCTGtccACCCAGtccGCCCTGtccAAAGACCCCAACGAGAAGCG
CGATCACATGGTCCTGCTGGAGTTCGTGACCGCCGCCGGGATCACTCTCGGCA
TGGACGAGCTATACAAGGAGCAGAAGCTGATCtccGAGGAGGATCTGTAAGCG
GCCGCCCTCCTCCTCCTTTCTTGTTCCTTTCACGTCGCCTTCTCGGTTGTAGCTG
GCAGACGACGAGTCTTACTTTTACGTGTACTTCTCTATAGATGATGTATGATCT
CTCTGCATGCGTGTTCGTGCATGTGTCCGTGTGTTGGGTACGCGTGGTACCCTG
CAGGAAGGAAGCTGAGTTGGCTGCTGCCACCGCTGAGCAATAACTAGTAATTA
CTAGCATAACCCCTTGGGGCCTCTAAACGGGTCTTGAGGGGGTTTTTTGCTGAA
AGGAGGACAGCTGATGATTGTCATGCTTGCCATCTGTTTTCTTGCAAGGTCAGA
GGAATTCGTAATCATGGTCATAGCTGTTTCCTGTGTGAAATTGTTATCCGCTCA
CAATTCCACACAACATACGAGCCGGAAGCATAAAGTGTAAAGCCTGGGGTGCC
TAATGAGTGAGCTAACTCACATTAATTGCGTTGCGCTCACTGCCCGCTTTCCAG
TCGGGAAACCTGTCGTGCCAGCTGCATTAATGAATCGGCCAACGCGCGGGGAG
AGGCGGTTTGCGTATTGGGCGCTCTTCCGCTTCCTCGCTCACTGACTCGCTGCG
CTCGGTCGTTCGGCTGCGGCGAGCGGTATCAGCTCACTCAAAGGCGGTAATAC
GGTTATCCACAGAATCAGGGGATAACGCAGGAAAGAACATGTGAGCAAAAGG
CCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGG
CGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCT
CCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGA
CACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACA
CTAGAAGAACAGTATTTGGTATCTGCGCTCTGCTGAAGCCAGTTACCTTCGGA
AAAAGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAGAGG
ATCCTTTGATCTTTTCTACGGGGTCTGACGCTCAGTGGAACGAAAACTCACGTT
AAGGGATTTTGGTCATGAGATTATCAAAAAGGATCTTCACCTAGATCCTTTTAA
ATTAAAAATGAAGTTTTAAATCAATCTAAAGTATATATGAGTAAACTTGGTCT
GACAGTTACCAATGCTTAATCAGTGAGGCACCTATCTCAGCGATCTGTCTAGTT
CGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACC
GGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCATTGCT
ACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGTTATC
ACTCATGGTTATGGCAGCACTGCATAATTCTCTTACTGTCATGCCATCCGTAAG
ATGCTTTTCTGTGACTGGTGAGTACTCAACCAAGTCATTCTGAGAATAGTGTAT
GCGGCGACCGAGTTGCTCTTGCCCGGCGTCAATACGGGATAATACCGCGCCAC
ATAGCAGAACTTTAAAAGTGCTCATCATTGGAAAACGTTCTTCGGGGCGAAAA
CTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTG
TCTCATGAGCGGATACATATTTGAATGTATTTAGAAAAATAAACAAATAGGGG
TTCCGCGCACATTTCCCCGAAAAGTGCCACCT
SEQ ID NO: 56 (translated protein sequence from 2AGC template)
MTVMYKVCKDIKHVSETMEISSKGEELFTGVVPILVELDGDVNGHKFSVSGEGEG
DATYGKLTLKFICTTGKLPVPWPTLVTTLTYGVQCFSRYPDHMKQHDFFKSAMPE
GYVQERTIFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLLPDNHYL
STQSALSKDPNEKRDHMVLLEFVTAAGITLGMDELYKEQKLISEEDL
SEQ ID NO: 57 (translated eGFP protein from 2AGC template (i.e., excluding 17 aas
from SITS))
MEISSKGEELFTGVVPILVELDGDVNGHKFSVSGEGEGDATYGKLTLKFICTTGKL
PVPWPTLVTTLTYGVQCFSRYPDHMKQHDFFKSAMPEGYVQERTIFFKDDGNYK
TRAEVKFEGDTLVNRIELKGIDFKEDGNILGHKLEYNYNSHNVYIMADKQKNGIK
VNFKIRHNIEDGSVQLADHYQQNTPIGDGPVLLPDNHYLSTQSALSKDPNEKRDH
MVLLEFVTAAGITLGMDELYKEQKLISEEDL
SEQ ID NO: 58 (modified SITS* DNA sequence)
TTTATTTTATTTTATTTTATTTTATTTTATTTTATTTTATTTTATTTTATTTTAATT
AATTTTATTTAACCATGACAGTAATGTATAAAGTCTGTAAAGACATTAAACACT
TAGGTGAAACC
SEQ ID NO: 59 (modified SITS* protein sequence)
MTVMYKVCKDIKHLGET
SEQ ID NO: 60 (1AGC codon eGFP ORF)
ATGACAGTAATGTATAAAGTCTGTAAAGACATTAAACACTTAGGTGAAACCATGGAG
ATCagcAAGGGAGAGGAGCTGTTCACAGGAGTGGTGCCGATCCTGGTAGAGCTG
GACGGAGACGTAAACGGGCACAAGTTCtcgGTGtcaGGAGAGGGAGAGGGAGAT
GCCACGTACGGGAAGCTGACACTGAAGTTCATCTGCACGACAGGAAAGCTGCC
AGTACCGTGGCCGACGCTGGTGACGACACTGACGTACGGAGTACAGTGCTTCtc
aCGGTACCCAGACCACATGAAGCAGCACGACTTCTTCAAGtcgGCTATGCCAGA
AGGGTACGTACAGGAGAGAACGATCTTCTTCAAGGACGACGGAAACTACAAG
ACACGGGCTGAGGTGAAGTTCGAGGGAGACACGCTGGTGAACAGGATCGAGC
TGAAGGGAATCGACTTCAAGGAGGACGGAAACATCCTGGGACACAAGCTGGA
GTACAACTACAACtcgCACAACGTATATATCATGGCCGACAAGCAGAAGAACGG
GATCAAGGTGAACTTCAAGATCCGGCACAACATCGAGGACGGAtcgGTACAGCT
GGCTGACCACTACCAGCAGAACACACCGATCGGAGACGGACCAGTACTGCTG
CCAGACAACCACTACCTGtcgACGCAGtcaGCTCTGtcgAAAGACCCGAACGAGAA
GAGAGATCACATGGTACTGCTGGAGTTCGTGACAGCCGCTGGAATCACGCTGG
GGATGGACGAGCTGTACAAGGAGCAGAAGCTGATCtcgGAGGAGGATCTGTAA
Wherein residues in italics are the 3′-SITS* sequence. This is followed by the eGFP
coding sequence. Ser codons in the ORF are shown in lower case: 1 AGC; 3 TCA; 7 TCG.
SEQ ID NO: 61 (1AGC codon template DNA sequence)
GACGTCTAATACGACTCACTATAGGGACATCTTAAGTTTATTTTATTTTATTTTA
TTTTATTTTATTTTATTTTATTTTATTTTATTTTAATTAATTTTATTTAACCATGAC
AGTAATGTATAAAGTCTGTAAAGACATTAAACACTTAGGTGAAACCATGGAGATCagc
AAGGGAGAGGAGCTGTTCACAGGAGTGGTGCCGATCCTGGTAGAGCTGGACG
GAGACGTAAACGGGCACAAGTTCtcgGTGtcaGGAGAGGGAGAGGGAGATGCCA
CGTACGGGAAGCTGACACTGAAGTTCATCTGCACGACAGGAAAGCTGCCAGTA
CCGTGGCCGACGCTGGTGACGACACTGACGTACGGAGTACAGTGCTTCtcaCGG
TACCCAGACCACATGAAGCAGCACGACTTCTTCAAGtcgGCTATGCCAGAAGGG
TACGTACAGGAGAGAACGATCTTCTTCAAGGACGACGGAAACTACAAGACAC
GGGCTGAGGTGAAGTTCGAGGGAGACACGCTGGTGAACAGGATCGAGCTGAA
GGGAATCGACTTCAAGGAGGACGGAAACATCCTGGGACACAAGCTGGAGTAC
AACTACAACtcgCACAACGTATATATCATGGCCGACAAGCAGAAGAACGGGATC
AAGGTGAACTTCAAGATCCGGCACAACATCGAGGACGGAtcgGTACAGCTGGCT
GACCACTACCAGCAGAACACACCGATCGGAGACGGACCAGTACTGCTGCCAG
ACAACCACTACCTGtcgACGCAGtcaGCTCTGtcgAAAGACCCGAACGAGAAGAGA
GATCACATGGTACTGCTGGAGTTCGTGACAGCCGCTGGAATCACGCTGGGGAT
GGACGAGCTGTACAAGGAGCAGAAGCTGATCtcgGAGGAGGATCTGTAAGCGG
CCGCCCTCCTCCTCCTTTCTTGTTCCTTTCACGTCGCCTTCTCGGTTGTAGCTGG
CAGACGACGAGTCTTACTTTTACGTGTACTTCTCTATAGATGATGTATGATCTC
TCTGCATGCGTGTTCGTGCATGTGTCCGTGTGTTGGGTACGCGTGGTACCCTGC
AGGAAGGAAGCTGAGTTGGCTGCTGCCACCGCTGAGCAATAACTAGTAATTAC
TAGCATAACCCCTTGGGGCCTCTAAACGGGTCTTGAGGGGGTTTTTTGCTGAAA
GGAGGACAGCTGATGATTGTCATGCTTGCCATCTGTTTTCTTGCAAGGTCAGAG
GAATTCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCT
AATGAGTGAGCTAACTCACATTAATTGCGTTGCGCTCACTGCCCGCTTTCCAGT
CGGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGC
CAGCAAAAGGCCAGGAACCGTAAAAAGGCCGCGTTGCTGGCGTTTTTCCATAG
GCTCCGCCCCCCTGACGAGCATCACAAAAATCGACGCTCAAGTCAGAGGTGGC
GAAACCCGACAGGACTATAAAGATACCAGGCGTTTCCCCCTGGAAGCTCCCTC
GTGCGCTCTCCTGTTCCGACCCTGCCGCTTACCGGATACCTGTCCGCCTTTCTC
CCTTCGGGAAGCGTGGCGCTTTCTCATAGCTCACGCTGTAGGTATCTCAGTTCG
GTGTAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGG
TATGTAGGCGGTGCTACAGAGTTCTTGAAGTGGTGGCCTAACTACGGCTACAC
TAGAAGAACAGTATTTGGTATCTGCGCTCTGCTGAAGCCAGTTACCTTCGGAA
AAAGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAGGA
TCCTTTGATCTTTTCTACGGGGTCTGACGCTCAGTGGAACGAAAACTCACGTTA
AGGGATTTTGGTCATGAGATTATCAAAAAGGATCTTCACCTAGATCCTTTTAAA
TTAAAAATGAAGTTTTAAATCAATCTAAAGTATATATGAGTAAACTTGGTCTG
ACAGTTACCAATGCTTAATCAGTGAGGCACCTATCTCAGCGATCTGTCTAGTTC
GTTCATCCATAGTTGCCTGACTCCCCGTCGTGTAGATAACTACGATACGGGAG
GGCTTACCATCTGGCCCCAGTGCTGCAATGATACCGCGAGACCCACGCTCACC
GGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCATTGCT
ACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGTTATC
ACTCATGGTTATGGCAGCACTGCATAATTCTCTTACTGTCATGCCATCCGTAAG
ATGCTTTTCTGTGACTGGTGAGTACTCAACCAAGTCATTCTGAGAATAGTGTAT
GCGGCGACCGAGTTGCTCTTGCCCGGCGTCAATACGGGATAATACCGCGCCAC
ATAGCAGAACTTTAAAAGTGCTCATCATTGGAAAACGTTCTTCGGGGCGAAAA
CTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTG
TCTCATGAGCGGATACATATTTGAATGTATTTAGAAAAATAAACAAATAGGGG
TTCCGCGCACATTTCCCCGAAAAGTGCCACCT
SEQ ID NO: 62 (translated protein sequence from 1AGC template)
MTVMYKVCKDIKHLGETMEISKGEELFTGVVPILVELDGDVNGHKFSVSGEGEGD
ATYGKLTLKFICTTGKLPVPWPTLVTTLTYGVQCFSRYPDHMKQHDFFKSAMPEG
YVQERTIFFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLPDNHYLS
TQSALSKDPNEKRDHMVLLEFVTAAGITLGMDELYKEQKLISEEDL
SEQ ID NO: 63 (translated eGFP protein from 1AGC template (i.e., excluding 17 aas
from SITS))
MEISKGEELFTGVVPILVELDGDVNGHKFSVSGEGEGDATYGKLTLKFICTTGKLP
VPWPTLVTTLTYGVQCFSRYPDHMKQHDFFKSAMPEGYVQERTIFFKDDGNYKT
RAEVKFEGDTLVNRIELKGIDFKEDGNILGHKLEYNYNSHNVYIMADKQKNGIKV
NFKIRHNIEDGSVQLADHYQQNTPIGDGPVLLPDNHYLSTQSALSKDPNEKRDHM
VLLEFVTAAGITLGMDELYKEQKLISEEDL
SEQ ID NO: 64 (1AGT codon eGFP ORF)
ATGACAGTAATGTATAAAGTCTGTAAAGACATTAAACACTTAGGTGAAACCATGGAG
ATCagtAAGGGCGAGGAGCTGTTCACCGGGGTGGTGCCCATCCTGGTCGAGCTG
GACGGCGACGTAAACGGCCACAAGTTCtcgGTGtcgGGCGAGGGCGAGGGCGAT
GCCACCTACGGCAAGCTGACCCTGAAGTTCATCTGCACCACCGGCAAGCTGCC
CGTGCCCTGGCCCACCCTGGTGACCACCCTGACCTACGGCGTGCAGTGCTTCtcg
CGGTACCCCGACCACATGAAGCAGCACGACTTCTTCAAGtcgGCCATGCCCGAA
GGCTACGTCCAGGAGCGGACCATCTTCTTCAAGGACGACGGCAACTACAAGAC
CCGGGCCGAGGTGAAGTTCGAGGGCGACACCCTGGTGAACCGGATCGAGCTG
AAGGGCATCGACTTCAAGGAGGACGGCAACATCCTGGGGCACAAGCTGGAGT
ACAACTACAACtcgCACAACGTCTATATCATGGCCGACAAGCAGAAGAACGGCA
TCAAGGTGAACTTCAAGATCCGGCACAACATCGAGGACGGCtcgGTGCAGCTGG
CCGACCACTACCAGCAGAACACCCCCATCGGCGACGGCCCCGTGCTGCTGCCC
GACAACCACTACCTGtcgACCCAGtcgGCCCTGtcgAAAGACCCCAACGAGAAGCG
GGATCACATGGTCCTGCTGGAGTTCGTGACCGCCGCCGGGATCACTCTGGGCA
TGGACGAGCTATACAAGGAGCAGAAGCTGATCtcgGAGGAGGATCTGTAA
Wherein residues in italics are the 3′-SITS* sequence. This is followed by the eGFP
coding sequence. Ser codons in the ORF are shown in lower case: 1 AGT; 10 TCG.
SEQ ID NO: 65 (1AGT codon template DNA sequence)
GACGTCTAATACGACTCACTATAGGGACATCTTAAGTTTATTTTATTTTATTTTA
TTTTATTTTATTTTATTTTATTTTATTTTATTTTAATTAATTTTATTTAACCATGA
CAGTAATGTATAAAGTCTGTAAAGACATTAAACACTTAGGTGAAACCATGGAG
ATCAGTAAGGGCGAGGAGCTGTTCACCGGGGTGGTGCCCATCCTGGTCGAGCT
GGACGGCGACGTAAACGGCCACAAGTTCTCGGTGTCGGGCGAGGGCGAGGGC
GATGCCACCTACGGCAAGCTGACCCTGAAGTTCATCTGCACCACCGGCAAGCT
GCCCGTGCCCTGGCCCACCCTGGTGACCACCCTGACCTACGGCGTGCAGTGCT
TCTCGCGGTACCCCGACCACATGAAGCAGCACGACTTCTTCAAGTCGGCCATG
CCCGAAGGCTACGTCCAGGAGCGGACCATCTTCTTCAAGGACGACGGCAACTA
CAAGACCCGGGCCGAGGTGAAGTTCGAGGGCGACACCCTGGTGAACCGGATC
GAGCTGAAGGGCATCGACTTCAAGGAGGACGGCAACATCCTGGGGCACAAGC
TGGAGTACAACTACAACTCGCACAACGTCTATATCATGGCCGACAAGCAGAAG
AACGGCATCAAGGTGAACTTCAAGATCCGGCACAACATCGAGGACGGCTCGGT
GCAGCTGGCCGACCACTACCAGCAGAACACCCCCATCGGCGACGGCCCCGTGC
TGCTGCCCGACAACCACTACCTGTCGACCCAGTCGGCCCTGTCGAAAGACCCC
AACGAGAAGCGGGATCACATGGTCCTGCTGGAGTTCGTGACCGCCGCCGGGAT
CACTCTGGGCATGGACGAGCTATACAAGGAGCAGAAGCTGATCTCGGAGGAG
GATCTGTAAGCGGCCGCCCTCCTCCTCCTTTCTTGTTCCTTTCACGTCGCCTTCT
CGGTTGTAGCTGGCAGACGACGAGTCTTACTTTTACGTGTACTTCTCTATAGAT
GATGTATGATCTCTCTGCATGCGTGTTCGTGCATGTGTCCGTGTGTTGGGTACG
CGTGGTACCCTGCAGGAAGGAAGCTGAGTTGGCTGCTGCCACCGCTGAGCAAT
AACTAGTAATTACTAGCATAACCCCTTGGGGCCTCTAAACGGGTCTTGAGGGG
GTTTTTTGCTGAAAGGAGGACAGCTGATGATTGTCATGCTTGCCATCTGTTTTC
TTGCAAGGTCAGAGGAATTCGTAATCATGGTCATAGCTGTTTCCTGTGTGAAAT
TGTTATCCGCTCACAATTCCACACAACATACGAGCCGGAAGCATAAAGTGTAA
AGCCTGGGGTGCCTAATGAGTGAGCTAACTCACATTAATTGCGTTGCGCTCACT
GCCCGCTTTCCAGTCGGGAAACCTGTCGTGCCAGCTGCATTAATGAATCGGCC
AACGCGCGGGGAGAGGCGGTTTGCGTATTGGGCGCTCTTCCGCTTCCTCGCTC
ACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGACGC
TCAAGTCAGAGGTGGCGAAACCCGACAGGACTATAAAGATACCAGGCGTTTCC
CCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGA
ACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGA
TTAGCAGAGCGAGGTATGTAGGCGGTGCTACAGAGTTCTTGAAGTGGTGGCCT
AACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAA
GGATCTCAAGAGGATCCTTTGATCTTTTCTACGGGGTCTGACGCTCAGTGGAAC
GAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGA
GTAAACTTGGTCTGACAGTTACCAATGCTTAATCAGTGAGGCACCTATCTCAGC
GATCTGTCTAGTTCGTTCATCCATAGTTGCCTGACTCCCCGTCGTGTAGATAAC
TACGATACGGGAGGGCTTACCATCTGGCCCCAGTGCTGCAATGATACCGCGAG
ACCCACGCTCACCGGCTCCAGATTTATCAGCAATAAACCAGCCAGCCGGAAGG
GCCGAGCGCAGAAGTGGTCCTGCAACTTTATCCGCCTCCATCCAGTCTATTAAT
TGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTC
ATTCAGCTCCGGTTCCCAACGATCAAGGCGAGTTACATGATCCCCCATGTTGTG
CAAAAAAGCGGTTAGCTCCTTCGGTCCTCCGATCGTTGTCAGAAGTAAGTTGG
CCGCAGTGTTATCACTCATGGTTATGGCAGCACTGCATAATTCTCTTACTGTCA
TGCCATCCGTAAGATGCTTTTCTGTGACTGGTGAGTACTCAACCAAGTCATTCT
GAGAATAGTGTATGCGGCGACCGAGTTGCTCTTGCCCGGCGTCAATACGGGAT
AATACCGCGCCACATAGCAGAACTTTAAAAGTGCTCATCATTGGAAAACGTTC
TTCGGGGCGAAAACTCTCAAGGATCTTACCGCTGTTGAGATCCAGTTCGATGT
AACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGG
CGACACGGAAATGTTGAATACTCATACTCTTCCTTTTTCAATATTATTGAAGCA
TTTATCAGGGTTATTGTCTCATGAGCGGATACATATTTGAATGTATTTAGAAAA
ATAAACAAATAGGGGTTCCGCGCACATTTCCCCGAAAAGTGCCACCT
SEQ ID NO: 66 (translated protein sequence from 1AGT template)
MTVMYKVCKDIKHLGETMEISKGEELFTGVVPILVELDGDVNGHKFSVSGEGEGD
ATYGKLTLKFICTTGKLPVPWPTLVTTLTYGVQCFSRYPDHMKQHDFFKSAMPEG
YVQERTIFFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLPDNHYLS
TQSALSKDPNEKRDHMVLLEFVTAAGITLGMDELYKEQKLISEEDL
SEQ ID NO: 67 (translated eGFP protein from 1AGT template (i.e., excluding 17 aas
from SITS))
MEISKGEELFTGVVPILVELDGDVNGHKFSVSGEGEGDATYGKLTLKFICTTGKLP
VPWPTLVTTLTYGVQCFSRYPDHMKQHDFFKSAMPEGYVQERTIFFKDDGNYKT
RAEVKFEGDTLVNRIELKGIDFKEDGNILGHKLEYNYNSHNVYIMADKQKNGIKV
NFKIRHNIEDGSVQLADHYQQNTPIGDGPVLLPDNHYLSTQSALSKDPNEKRDHM
VLLEFVTAAGITLGMDELYKEQKLISEEDL
SEQ ID NO: 68 (1AGG codon eGFP ORF)
ATGACAGTAATGTATAAAGTCTGTAAAGACATTAAACACTTAGGTGAAACCATGGAG
ATCaggAAGGGCGAGGAGCTGTTCACCGGGGTGGTGCCCATCCTGGTCGAGCTG
GACGGCGACGTAAACGGCCACAAGTTCTCGGTGTCGGGCGAGGGCGAGGGCG
ATGCCACCTACGGCAAGCTGACCCTGAAGTTCATCTGCACCACCGGCAAGCTG
CCCGTGCCCTGGCCCACCCTGGTGACCACCCTGACCTACGGCGTGCAGTGCTTC
TCGcggTACCCCGACCACATGAAGCAGCACGACTTCTTCAAGTCGGCCATGCCC
GAAGGCTACGTCCAGGAGcggACCATCTTCTTCAAGGACGACGGCAACTACAAG
ACCcggGCCGAGGTGAAGTTCGAGGGCGACACCCTGGTGAACcggATCGAGCTG
AAGGGCATCGACTTCAAGGAGGACGGCAACATCCTGGGGCACAAGCTGGAGT
ACAACTACAACTCGCACAACGTCTATATCATGGCCGACAAGCAGAAGAACGG
CATCAAGGTGAACTTCAAGATCcggCACAACATCGAGGACGGCTCGGTGCAGCT
GGCCGACCACTACCAGCAGAACACCCCCATCGGCGACGGCCCCGTGCTGCTGC
CCGACAACCACTACCTGTCGACCCAGTCGGCCCTGTCGAAAGACCCCAACGAG
AAGcggGATCACATGGTCCTGCTGGAGTTCGTGACCGCCGCCGGGATCACTCTG
GGCATGGACGAGCTATACAAGGAGCAGAAGCTGATCTCGGAGGAGGATCTGT
AA
Wherein residues in italics are the 3′-SITS* sequence. This is followed by the eGFP
coding sequence. Arg codons in the ORF are shown in lower case: 1 AGG; 6 CGG.
SEQ ID NO: 69 (1AGG codon template DNA sequence)
GACGTCTAATACGACTCACTATAGGGACATCTTAAGTTTATTTTATTTTATTTTA
TTTTATTTTATTTTATTTTATTTTATTTTATTTTAATTAATTTTATTTAACCATGA
CAGTAATGTATAAAGTCTGTAAAGACATTAAACACTTAGGTGAAACCATGGAG
ATCAGGAAGGGCGAGGAGCTGTTCACCGGGGTGGTGCCCATCCTGGTCGAGCT
GGACGGCGACGTAAACGGCCACAAGTTCTCGGTGTCGGGCGAGGGCGAGGGC
GATGCCACCTACGGCAAGCTGACCCTGAAGTTCATCTGCACCACCGGCAAGCT
GCCCGTGCCCTGGCCCACCCTGGTGACCACCCTGACCTACGGCGTGCAGTGCT
TCTCGCGGTACCCCGACCACATGAAGCAGCACGACTTCTTCAAGTCGGCCATG
CCCGAAGGCTACGTCCAGGAGCGGACCATCTTCTTCAAGGACGACGGCAACTA
CAAGACCCGGGCCGAGGTGAAGTTCGAGGGCGACACCCTGGTGAACCGGATC
GAGCTGAAGGGCATCGACTTCAAGGAGGACGGCAACATCCTGGGGCACAAGC
TGGAGTACAACTACAACTCGCACAACGTCTATATCATGGCCGACAAGCAGAAG
AACGGCATCAAGGTGAACTTCAAGATCCGGCACAACATCGAGGACGGCTCGGT
GCAGCTGGCCGACCACTACCAGCAGAACACCCCCATCGGCGACGGCCCCGTGC
TGCTGCCCGACAACCACTACCTGTCGACCCAGTCGGCCCTGTCGAAAGACCCC
AACGAGAAGCGGGATCACATGGTCCTGCTGGAGTTCGTGACCGCCGCCGGGAT
CACTCTGGGCATGGACGAGCTATACAAGGAGCAGAAGCTGATCTCGGAGGAG
GATCTGTAAGCGGCCGCCCTCCTCCTCCTTTCTTGTTCCTTTCACGTCGCCTTCT
CGGTTGTAGCTGGCAGACGACGAGTCTTACTTTTACGTGTACTTCTCTATAGAT
GATGTATGATCTCTCTGCATGCGTGTTCGTGCATGTGTCCGTGTGTTGGGTACG
CGTGGTACCCTGCAGGAAGGAAGCTGAGTTGGCTGCTGCCACCGCTGAGCAAT
AACTAGTAATTACTAGCATAACCCCTTGGGGCCTCTAAACGGGTCTTGAGGGG
GTTTTTTGCTGAAAGGAGGACAGCTGATGATTGTCATGCTTGCCATCTGTTTTC
TTGCAAGGTCAGAGGAATTCGTAATCATGGTCATAGCTGTTTCCTGTGTGAAAT
TGTTATCCGCTCACAATTCCACACAACATACGAGCCGGAAGCATAAAGTGTAA
AGCCTGGGGTGCCTAATGAGTGAGCTAACTCACATTAATTGCGTTGCGCTCACT
GCCCGCTTTCCAGTCGGGAAACCTGTCGTGCCAGCTGCATTAATGAATCGGCC
AACGCGCGGGGAGAGGCGGTTTGCGTATTGGGCGCTCTTCCGCTTCCTCGCTC
ACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGACGC
TCAAGTCAGAGGTGGCGAAACCCGACAGGACTATAAAGATACCAGGCGTTTCC
CCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGA
ACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGA
TTAGCAGAGCGAGGTATGTAGGCGGTGCTACAGAGTTCTTGAAGTGGTGGCCT
AACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAA
GGATCTCAAGAGGATCCTTTGATCTTTTCTACGGGGTCTGACGCTCAGTGGAAC
GAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGA
GTAAACTTGGTCTGACAGTTACCAATGCTTAATCAGTGAGGCACCTATCTCAGC
GATCTGTCTAGTTCGTTCATCCATAGTTGCCTGACTCCCCGTCGTGTAGATAAC
TACGATACGGGAGGGCTTACCATCTGGCCCCAGTGCTGCAATGATACCGCGAG
ACCCACGCTCACCGGCTCCAGATTTATCAGCAATAAACCAGCCAGCCGGAAGG
GCCGAGCGCAGAAGTGGTCCTGCAACTTTATCCGCCTCCATCCAGTCTATTAAT
TGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTC
ATTCAGCTCCGGTTCCCAACGATCAAGGCGAGTTACATGATCCCCCATGTTGTG
CAAAAAAGCGGTTAGCTCCTTCGGTCCTCCGATCGTTGTCAGAAGTAAGTTGG
CCGCAGTGTTATCACTCATGGTTATGGCAGCACTGCATAATTCTCTTACTGTCA
TGCCATCCGTAAGATGCTTTTCTGTGACTGGTGAGTACTCAACCAAGTCATTCT
GAGAATAGTGTATGCGGCGACCGAGTTGCTCTTGCCCGGCGTCAATACGGGAT
AATACCGCGCCACATAGCAGAACTTTAAAAGTGCTCATCATTGGAAAACGTTC
TTCGGGGCGAAAACTCTCAAGGATCTTACCGCTGTTGAGATCCAGTTCGATGT
AACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGG
CGACACGGAAATGTTGAATACTCATACTCTTCCTTTTTCAATATTATTGAAGCA
TTTATCAGGGTTATTGTCTCATGAGCGGATACATATTTGAATGTATTTAGAAAA
ATAAACAAATAGGGGTTCCGCGCACATTTCCCCGAAAAGTGCCACCT
SEQ ID NO: 70 (translated protein sequence from 1AGG template)
MTVMYKVCKDIKHLGETMEIRKGEELFTGVVPILVELDGDVNGHKFSVSGEGEGD
ATYGKLTLKFICTTGKLPVPWPTLVTTLTYGVQCFSRYPDHMKQHDFFKSAMPEG
YVQERTIFFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLPDNHYLS
TQSALSKDPNEKRDHMVLLEFVTAAGITLGMDELYKEQKLISEEDL
SEQ ID NO: 71 (translated eGFP protein from 1AGG template (i.e., excluding 17 aas
from SITS))
MEIRKGEELFTGVVPILVELDGDVNGHKFSVSGEGEGDATYGKLTLKFICTTGKLP
VPWPTLVTTLTYGVQCFSRYPDHMKQHDFFKSAMPEGYVQERTIFFKDDGNYKT
RAEVKFEGDTLVNRIELKGIDFKEDGNILGHKLEYNYNSHNVYIMADKQKNGIKV
NFKIRHNIEDGSVQLADHYQQNTPIGDGPVLLPDNHYLSTQSALSKDPNEKRDHM
VLLEFVTAAGITLGMDELYKEQKLISEEDL
SEQ ID NO: 72 (T7 promoter)
TAATACGACTCACTATA
SEQ ID NO: 73 (the reverse complement of the T7 promoter)
TATAGTGAGTCGTATTA

1-48. (canceled)

49. An oligonucleotide that hybridises to a tRNA of interest when said tRNA is in a folded state, thereby disrupting the function of the tRNA.

50. The oligonucleotide of claim 49, wherein the oligonucleotide hybridises to:
(a) the region of the tRNA of interest spanning from the anticodon stem-loop to the variable loop;
(b) the region of the tRNA of interest spanning from the anticodon stem-loop to the T stem-loop;
(c) the region of the tRNA of interest spanning from the T stem-loop to the 3′CCA-end; or
(d) the region of the tRNA of interest spanning from the D stem-loop to the anticodon stem-loop.

51. The oligonucleotide of claim 49, wherein:
(a) the oligonucleotide hybridises to a region of the tRNA of interest corresponding to nucleotides N34-47j or N8-47k of E. coli tRNA^{Ser}_{GCU}; N31-53 of E. coli tRNA^{Arg}_{CCU}; or N56-76 of L. tarentolae tRNA^{Ser}_{GCU}; or
(b) the oligonucleotide hybridises to the tRNA of interest in a folded state with a K_{d} of between 0.1-100 nM.

52. The oligonucleotide of claim 49, wherein the oligonucleotide:
(a) changes the structure of the tRNA, optionally disrupting the folded state of the tRNA;
(b) disrupts the translational function of the tRNA; and/or
(c) reduces translation of an mRNA comprising a codon that is recognised by the anticodon of the tRNA of interest.

53. The oligonucleotide of claim 49, wherein:
(a) the oligonucleotide comprises or consists of ribonucleotides or modified ribonucleotides; optionally wherein the modified ribonucleotides are selected from 2′-O-methylated ribonucleotides, 2′-fluorated ribonucleotides and locked nucleic acid (LNA) nucleotides; or
(b) the oligonucleotide comprises a sequence selected from any one of SEQ ID NOs 1-31, or a variant thereof; or
(c) the oligonucleotide is between 10-60 nucleotides in length; or
(d) the oligonucleotide hybridises by Watson-Crick and/or wobble-base pairing to the tRNA of interest; or
(e) the oligonucleotide hybridises to a target sequence in the tRNA of interest that is between about 10-60 base pairs in length.

54. The oligonucleotide of claim 49, wherein the tRNA of interest recognises a codon encoding a six-fold degenerate amino acid, such as a codon encoding arginine, leucine or serine, or the initiator methionine codon; optionally wherein:
(a) the tRNA of interest is selected from tRNA^{Arg}, tRNA^{Leu}, tRNA^{Ser} or tRNA^{iMet}; or
(b) the tRNA of interest is selected from tRNA^{Ser}_{GCU}, tRNA^{Ser}_{GGA}, tRNA^{Ser}_{UGA}, tRNA^{Arg}_{CCU}, tRNA^{Arg}_{CCG}, initiator tRNA^{iMet}, tRNA^{Ser}_{CGA}, tRNA^{Arg}_{UCU}, tRNA^{Leu}_{CAA}, tRNA^{Leu}_{CAG} and tRNA^{Leu}_{GAG}; or
(c) the tRNA of interest comprises the sequence of any one of SEQ ID NOs 32-44, or a variant thereof.

55. A composition of matter comprising:
A. a composition or kit comprising:
(a) an oligonucleotide that hybridises to a tRNA of interest when said tRNA is in a folded state, thereby disrupting the function of the tRNA; and
(b) one or more of:
(i) a tRNA that (a) recognises the same codon as the tRNA of interest, and (b) is linked to an unnatural amino acid;
(ii) one or more tRNAs comprising the tRNA of interest; and/or
(iii) one or more translation reagents;
or
B. a composition or kit comprising:
(a) an oligonucleotide that hybridises to a tRNA of interest when said tRNA is in a folded state, thereby disrupting the function of the tRNA; and
(b) one or more of:
(i) an orthogonal tRNA that recognises the same codon as the tRNA of interest;
(ii) an unnatural amino acid suitable for coupling to the tRNA;
(iii) an orthogonal aminoacyl-tRNA synthetase suitable for charging the orthogonal tRNA with the unnatural amino acid;
(iv) one or more tRNAs, comprising the tRNA of interest; and/or
(v) one or more translation reagents;
or
C. a vector suitable for expressing an oligonucleotide that hybridises to a tRNA of interest when said tRNA is in a folded state, thereby disrupting the function of the tRNA, optionally wherein the vector is a plasmid;
or
D. a cell comprising a vector suitable for expressing an oligonucleotide that hybridises to a tRNA of interest when said tRNA is in a folded state, thereby disrupting the function of the tRNA, optionally wherein the vector is a plasmid;
or
E. a cell comprising an oligonucleotide that hybridises to a tRNA of interest when said tRNA is in a folded state, thereby disrupting the function of the tRNA;
or
F. a lysate prepared from said cell, said lysate comprising an oligonucleotide that hybridises to a tRNA of interest when said tRNA is in a folded state, thereby disrupting the function of the tRNA;
or
G. a composition comprising a lysate prepared from a cell comprising one or more of:
(a) an mRNA comprising a codon that is recognised by a tRNA of interest;
(b) an orthogonal tRNA that recognises the same codon as the tRNA of interest;
(c) an unnatural amino acid; and/or
(d) an orthogonal aminoacyl-tRNA synthetase suitable for charging the orthogonal tRNA with the unnatural amino acid;
wherein the composition further comprises an oligonucleotide that hybridises to a tRNA of interest when said tRNA is in a folded state, thereby disrupting the function of the tRNA.

56. The composition of matter according to claim 55 A or B, comprising:
(a) a plurality of tRNAs comprising tRNAs suitable for incorporation of all of the twenty canonical amino acids; optionally wherein the plurality of tRNAs comprises a full complement of naturally occurring tRNAs; or
(b) a plurality of amino acids, optionally comprising all twenty canonical amino acids; or
(c) ribosomes; or
(d) protein factors for translation; optionally wherein the protein factors comprise initiation, elongation and release factors; or
(e) a cell lysate providing said one or more tRNAs, ribosomes, protein factors for translation; or
(f) an mRNA comprising a codon that is recognised by the tRNA of interest; or
(g) a vector encoding an mRNA comprising a codon that is recognised by the tRNA of interest, optionally wherein the composition or kit further comprises protein factors for transcription, such as T7 polymerase.

57. The composition of matter according to claim 55 D or E, further comprising one or more of:
(a) an mRNA comprising a codon that is recognised by a tRNA of interest;
(b) an orthogonal tRNA that recognises the same codon as the tRNA of interest;
(c) an unnatural amino acid; and/or
(d) an orthogonal aminoacyl-tRNA synthetase suitable for charging the orthogonal tRNA with the unnatural amino acid.

58. The composition of matter according to claim 55 D or E, expressing said oligonucleotide under the control of an inducible promoter.

59. An in vitro method for:
A. disrupting function of a tRNA of interest, the method comprising contacting the tRNA in a folded state with an oligonucleotide that hybridises to a tRNA of interest when said tRNA is in a folded state, thereby disrupting the function of the tRNA; or
B. producing a polypeptide comprising at least one unnatural amino acid, wherein the method comprises incubating:
(a) an mRNA comprising a codon that is recognised by a tRNA of interest;
(b) an oligonucleotide that hybridises to a tRNA of interest when said tRNA is in a folded state, thereby disrupting the function of the tRNA; and
(c) a tRNA that (i) recognises the same codon as the tRNA of interest, and (ii) is linked to an unnatural amino acid;
under conditions suitable for translation of said mRNA; or
C. producing a polypeptide comprising at least one unnatural amino acid, wherein the method comprises incubating:
(a) an mRNA comprising a codon that is recognised by a tRNA of interest;
(b) an oligonucleotide that hybridises to a tRNA of interest when said tRNA is in a folded state, thereby disrupting the function of the tRNA;
(c) an orthogonal tRNA that recognises the same codon as the tRNA of interest;
(d) an unnatural amino acid; and
(e) an orthogonal aminoacyl-tRNA synthetase suitable for charging the orthogonal tRNA with the unnatural amino acid;
under conditions suitable for translation of said mRNA; or
D. producing a polypeptide comprising at least one unnatural amino acid, wherein the method comprises in a first step incubating:
(a) an oligonucleotide that hybridises to a tRNA of interest when said tRNA is in a folded state, thereby disrupting the function of the tRNA; and
(b) one or more tRNAs comprising the tRNA of interest, optionally a full complement of naturally occurring tRNAs comprising the tRNA of interest;
and in a second step incubating:
(c) the mixture resulting from the first step;
(d) an mRNA comprising a codon that is recognised by a tRNA of interest; and either:
(e) a tRNA that (i) recognises the same codon as the tRNA of interest and (ii) is linked to an unnatural amino acid; or
(e) an orthogonal tRNA that recognises the same codon as the tRNA of interest; an unnatural amino acid; and an orthogonal aminoacyl-tRNA synthetase suitable for charging the orthogonal tRNA with the unnatural amino acid;
under conditions suitable for translation of said mRNA; or
E. producing a polypeptide comprising at least one unnatural amino acid, wherein the method comprises incubating together:
(a) the lysate prepared from a composition of matter according to claim 55 D or E, said lysate comprising an oligonucleotide that hybridises to a tRNA of interest when said tRNA is in a folded state, thereby disrupting the function of the tRNA;
(b) an mRNA comprising a codon that is recognised by the tRNA of interest; and
(c) a tRNA that (i) recognises the same codon as the tRNA of interest, and (ii) is linked to an unnatural amino acid;
under conditions suitable for translation of said mRNA; or
F. producing a polypeptide comprising at least one unnatural amino acid, wherein the method comprises incubating the composition comprising a composition of matter according to claim 55 F under conditions suitable for translation of said mRNA.

60. The method for disrupting function of a tRNA of interest of claim 59, wherein:
(a) said contacting occurs in solution, optionally wherein the contacting occurs in water, a buffered solution or a cell lysate; or
(b) said contacting is carried out under non-denaturing conditions, optionally under physiological conditions, such as wherein the physiological conditions comprise a temperature of about 20° C. to about 40° C. and/or a pH of about 6 to about 8.

61. The method for producing a polypeptide comprising at least one unnatural amino acid of claim 59, wherein:
(a) the mRNA is produced by transcription of a DNA template and/or the oligonucleotide is produced by transcription of a DNA polynucleotide; or
(b) said incubating is carried out under non-denaturing conditions, optionally under physiological conditions, such as wherein the physiological conditions comprise a temperature of about 20° C. to about 40° C. and/or a pH of about 6 to about 8; or
(c) the translation reaction is performed in the presence of the tRNA of interest; or
(d) the tRNA is orthogonal to the endogenous amino acid charging system; or
(e) the tRNA and the aminoacyl-tRNA synthetase are orthogonal to the endogenous amino acid charging system, optionally wherein the orthogonal tRNA and the aminoacyl-tRNA synthetase form an orthogonal pair; or
(f) the tRNA or the orthogonal tRNA comprises the sequence of any one of SEQ ID NOs 47-50, or a variant thereof; or
(g) the unnatural amino acid is selected from p-azidophenylalanine, n-propargyllysine, selenocysteine, N-chloroacetyl-methionine, p-propargyloxyphenylalanine, fluorophores such as BODIPY FL, any N-methylated amino acid, such as N-methyl-valine, N-methyl-leucine, any D-amino acid, such as D-serine or D-tyrosine.

62. The method for producing a polypeptide comprising at least one unnatural amino acid of claim 59, wherein the tRNA of interest recognises a codon encoding a six-fold degenerate amino acid, such as a codon encoding arginine, leucine or serine, or the initiator methionine codon;
optionally wherein:
(a) the tRNA of interest is selected from tRNA^{Arg}, tRNA^{Leu}, tRNA^{Ser} or tRNA^{iMet}; or
(b) the tRNA of interest is selected from tRNA^{Ser}_{GCU}, tRNA^{Ser}_{GGA}, tRNA^{Ser}_{UGA}, tRNA^{Arg}_{CCU}, tRNA^{Arg}_{CCG}, initiator tRNA^{iMet}, tRNA^{Ser}_{CGA}, tRNA^{Arg}_{UCU}, tRNA^{Leu}_{CAA}, tRNA^{Leu}_{CAG} and tRNA^{Leu}_{GAG}; or
(c) the tRNA of interest comprises the sequence of any one of SEQ ID NOs 32-44, or a variant thereof.

63. A composition of matter comprising a polypeptide comprising one or more unnatural amino acids produced according to the method for producing a polypeptide comprising at least one unnatural amino acid of claim 59.
